(19) United States
(12) Patent Application Publication
(10) Pub. No.: US 2006/0052396 A1
(43) Pub. Date: Mar. 9, 2006

(54) Arylamines for the treatment of conditions associated with gsk-3

(76) Inventors: Stefan Berg, Sodertalje (SE); Sven Hellberg, Sodertalje (SE)

Correspondence Address: WHITE & CASE LLP;PATENT DEPARTMENT, 1155 AVENUE OF THE AMERICAS, NEW YORK, NY, 10036, US

(21) Appl. No.: 10/481,721

(22) PCT Filed: Jul. 3, 2002
(86) PCT No.: PCT/SE02/01339

§ 371 Date: Dec. 22, 2003

(30) Foreign Application Priority Data
Jul. 5, 2001 (SE) 0102439-7

(51) Int. Cl. A61K 31/4965; A61K 31/4439; C07D 403/02; C07D 401/02

(52) U.S. Cl. 514/255.06; 514/340; 514/352; 544/405; 544/406; 546/268.1; 546/308

(57) ABSTRACT

The present invention relates to new compounds of formula (I) wherein Z, Y, X, P, Q, R, R¹, R², R³, R⁴, R⁵, R⁶, R⁷, R⁸, R⁹, R¹⁰, R¹¹, R¹², A, m and n are defined as in any one of claims 1 to 3, a process for their preparation and new intermediates prepared therein, pharmaceutical formulations containing said therapeutically active compounds and to the use of said active compounds for the treatment of conditions associated with glycogens synthase kinase-3 (GSK3).

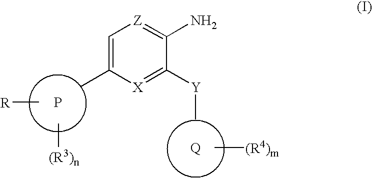

### FIELD OF THE INVENTION

The present invention relates to new compounds of formula I, as a free base or a pharmaceutically acceptable salt thereof, to pharmaceutical formulations containing said compounds and to the use of said compounds in therapy. The present invention further relates the process for the preparation of compounds of formula I and to new intermediates prepared therein.

An object of the invention is to provide compounds of formula I for therapeutic use, especially compounds that are useful for the prevention and/or treatment of conditions associated-with glycogen synthgas kinase-3 (GSK3) in mammals including man. Particularly compounds of formula I exhibiting inhibition of GSK-3.

It is also an object of the invention to provide compounds with a therapeutic effect after oral administration.

### BACKGROUND OF THE INVENTION

Glycogen synthase kinase 3 (GSK3) is a serine/threonine protein kin ase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

Alzheimer's Disease (AD) Dementias, and Taupathies.

AD is characterized by cognitive decline, cholinergic dysfunction and neuronal death, neurofibrillary tangles and senile plaques consisting of amyloid-β deposits. The sequence of these events in AD is unclear, but believed to be related. Glycogen synthase kinase 3β(GSK3β) or Tau (τ) phosphorylating kinase selectively phosphorylates the microtubule associated protein τ in neurons at sites that are hyperphosphorylated in AD brains. Hyperphosphorylated protein τ has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of τ and a paired helical filaments-like state via induction of GSK3β activity, followed by disruption of axonal transport and neuronal death (Imahori and Uchida., J. Biochem 121:179-188, 1997). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 93:2719-2723, 1996). Acetyl-Co-A is critical for the synthesis of acetylcholine, a neurotransmitter with cognitive functions. Thus, GSK3β inhibition may have beneficial effects in progression as well as the cognitive deficits associated with Alzheimer's disease and other above-referred to diseases.

Chronic and Acute Neurodegenerative Diseases.

Growth factor mediated activation of the PI3K/Akt pathway has been shown to play a key role in neuronal survival. The activation-of this pathway results in GSK3β inhibition. Recent studies (Bhat et. al., PNAS 97:11074-11079 (2000)) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as Alzheimer's Disease, Parkinson's Disease, amyotrophic lateral sclerosis, Huntington's Disease and HIV dementia, ischemic stroke and head trauma. Lithium was neuroprotective in inhibiting apoptosis in cells and in the brain at doses that resulted in the inhibition of GSK3β. Thus GSK3β inhibitors could be useful in attenuating the course of neurodegenerative diseases.

Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The recent discovery that lithium inhibits GSK3 at therapeutic concentrations has raised the possibility that this enzyme represents a key target of lithium's action in the brain (Stambolic et al., Curr. Biol. 6:1664-1668, 1996; Klein and Melton; PNAS 93:8455-8459, 1996). Inhibition of GSK3β may therefore be of therapeutic relevance in the treatment of BD as well as in AD patients that have affective disorders.

Schizophrenia

GSK3 is involved in signal transduction cascades of multiple cellular processes, particularly during neural development. Kozlovsky et al (Am J Psychiatry 2000May;157(5):831-3) found that GSK3β levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have-been reported in patients exhibiting schizophrenia (Cotter et al. Neuroreport 9:1379-1383 (1998)),

Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February;49(2):263-71). Inhibition of GSK3 increases the activity of glycogen synthase thereby decreasing glucose levels by its conversion to glycogen. GSK3 inhibition may therefore be of therapeutic relevance in the treatment of Type I and Type II diabetes and diabetic neuropathy.

Hair Loss

GSK3 phosphorylates and degrades β-catenin. β-catenin is an effector of the pathway for keratonin synthesis. β-catenin stabilisation may be lead to increase hair development. Mice. expressing a stabilised β-catenin by mutation of sites phosphorylated by GSK3 undergo a process resembling de novb hair morphogenesis (Gat et al., Cell 1998 Nov. 25;95 (5): 605-14)). The new follicles formed sebaceous glands and dermal papilla, normally established only in embryogenesis. Thus GSK3 inhibition may offer treatment for baldness.

Oral Contraceptives

Vijajaraghavan et al. (Biol Reprod 2000 June; 62 (6):1647-54) reported that GSK3 is high in motile versus immotile sperm. Immunocytochemistry revealed that GSK3 is present in the flagellum and the anterior portion of the sperm head. These data suggest, that GSK3 could be a key element underlying motility initiation in the epididymis and regulation of mature sperm function. Inhibitors of GSK3 could be useful as contraceptives for males.

### DISCLOSURE OF THE INVENTION

The object of the present invention is to provide compounds having a selective inhibiting effect at GSK3 as well as having a good bioavailability.

Accordingly, the present invention provides a compound of formula I 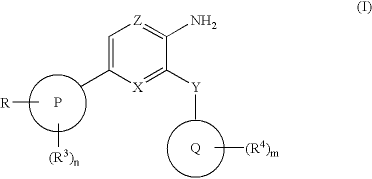
wherein:
Z is CH or N;
Y is CONR⁵, NR⁵CO, SO₂NR⁵, NR⁵SO₂, CH₂NR⁵, NR⁵CH₂, NR⁵CONR⁵, C₁₋₆alkylene, CH₂CO, COCH₂, CH═CH, OCH₂ or CH₂O;
X is CH or N;
P is phenyl or a 5 or 6 membered heteroaromatic ring containing one or more heteroatoms selected from N, O or S and said phenyl ring or 5 or 6 membered heteroaromatic ring may optionally be fused with a 5 or 6 membered saturated, partially saturated or unsaturated ring containing one or more atoms selected from C, N, O or S;
Q is phenyl or a 5 or 6 membered heteroaromatic ring containing one or more heteroatoms selected from N, O or S of which at least one atom is selected from nitrogen;
R is CHO, fluoromethoxy, difluoromethoxy, trifluoromethoxy, C₀₋₆alkyl(SO₂)NR¹R², OC₀₋₆alkyl(SO₂)NR¹¹R¹²alkyl(SO)NR¹R², C₁₋₆alkyl(SO)NR¹R², C₀₋₆alkylNR¹(SO)R², OC₁₋₆aykylNR¹(SO)R², C₀₋₆alkylNR¹(SO₂)NR¹R², OC₁₋₆alkylNR¹(SO₂)R², C₀₋₆alkyl(SO₂)C₁₋₆alkylNR¹R², OC₀₋₆alkyl(SO₂)C₁₋₆alkylNR¹R², C₀₋₆alkyl(SO)C₁₋₆alkylNR¹R², OC₁₋₆alkyl(SO)C₁₋₆alkylNR¹R², C₀₋₆alkylSC₁₋₆alkylNR¹R², OC₁₋₆alkylSC₁₋₆alkylNR¹R², OC₁₋₆alkylOC₁₋₆alkyl, C₁₋₆alkylOC₁₋₆alkylNR¹R², OC₁₋₆alkylOC₁₋₆alkylNR¹R², C₀₋₆alkylCONR¹⁰R¹¹, OC₀₋₆kylCONR¹R², OC₁₋₆alkylNR¹R², C₀₋₆alkylNR¹⁰(CO)R¹¹, OC₁₋₆alkylNR¹(CO)R², CO₀₋₆alkylNR¹(CO)R², C₀₋₆alkylNR¹¹(CO)R¹⁰, C₀₋₆alkylCOR¹¹, OC₁₋₆alkylCOR¹, C₀₋₆alkylNR¹⁰OR¹¹, C₀₋₆alkylO(CO)R¹¹, OC₁₋₆alkylO(CO)R¹, C₀₋₆alkylC(NR¹⁰)NR¹⁰R¹¹, C₀₋₆alkylC(NR¹¹)N(R¹⁰)₂, OC₀₋₆alkylC(NR¹)NR¹R², C₀₋₆alkylNR¹⁰(CO)OR¹¹, OC₁₋₆alkylNR¹(CO)OR², C₀₋₆alkylNR¹¹(CO)OR¹⁰, OC₁₋₆alkylCN, NR¹OR², C₀₋₆alkyl(CO)OR⁸, OC₁₋₆alkyl(CO)OR¹, NR¹ (CO)NR¹R², NR¹(CO)(CO)R²,NR¹(CO)(CO)NR¹R²,OR¹² or SO₃R¹;
R¹ and R² are independently selected from hydrogen, C₁₋₆alkyl, C₂₋₆alkenyl, C₂₋₆alkynyl, C₀₋₆alkylC₃₋₆cycloalkyl, (CO)OR⁸, C₀₋₆alkylheterocycloalkyl, C₁₋₆alkylNR⁶R⁷, C₀₋₆alkylaryl and C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylheterocycloalkyl, C₀₋₆alkylaryl C₀₋₆alkylheteroaryl may be substituted by one or more A;
R¹ and R² may together form a substituted 5, 6 or 7 membered heterocyclic ring containing one or more heteroatoms selected from N, O or S, which heterocyclic ring may be optionally substituted by A;
R³ and R⁴ are independently selected from halo, nitro, CHO, C₀₋₆alkylCN, OC₁₋₆alkylCN, C₀₋₆alkylOR⁶, OC₁₋₆alkylOR⁶, fluoromethyl, difluoromethyl, trifluoromethyl, fluoromethoxy, difluoromethoxy, trifluoromethoxy, C₀₋₆alkyNR⁶R⁷, OC₁₋₆alkylNR⁶R⁷, OC₁₋₆alkylOC₁₋₆alkylNR⁶R⁷, NR⁶OR⁷ C₀₋₆alkylCO₂R⁶, OC₁₋₆alkylCO₂R⁶, C₀₋₆alkylCONR⁶R⁷, OC₁ alkylCONR⁶R⁷, OC₁₋₆alkylNR⁶(CO)R⁷, C₀₋₆alkylNR⁶(CO)R^{7, O(CO)NR}⁶R⁷, NR⁶(CO)OR⁷, NR⁶(CO)NR⁶R⁷, O(CO)OR⁶, o(CO)R⁶, C₀₋₆alkylCOR⁶, OC₁₋₆alkylCOR⁶, NR⁶(CO)(CO)R⁶, NR⁶(CO)(CO)NR⁶R⁷, SR⁶, C₀₋₆alkyl(SO₂)NR⁶R⁷, OC₁₋₆alkylNR⁶(SO₂)R⁷, OC₀₋₆alkyl(SO₂)NR⁶R⁷, C₀₋₆alkyl(SO)NR⁶R⁷, OC₁₋₆alkyl(SO)NR⁶R⁷, SO₃R⁶, C₀₋₆alkylNR⁶(SO₂)NR⁶R⁷, C₀₋₆alkylNR⁶(SO)R⁷, OC₁₋₆alkylNR⁶(SO)R⁷, OC₀₋₆alkylSO₂R⁶, C₀₋₆alkylSO₂R⁶, C₀₋₆alkylSOR⁶, C₁₋₆alkyl, C₂₋₆alkenyl, C₂₋₆alkynyl, C₀₋₆alkylC₃₋₆cycloalkyl, C₀₋₆alkylaryl and Co-alkylheteroaryl, wherein any C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl may be optionally substituted by one or more A;
m is 0, 1, 2, 3 or 4;
n is 0, 1, 2, 3 or 4;
R⁵ is hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₁₋₆alkylNR⁶R⁷ or C₁₋₆alkylCONR⁶R⁷;
R⁶ and R⁷ are independently selected from hydrogen, C₁₋₆alkyl, (CO)OR⁸,
R₂₋₆alkenyl, C₂₋₆alkynyl, C₀₋₆alkylC₃₋₆cycloalkyl, C₀₋₆alkylaryl, C₀₋₆alkylheteroaryl and C₁₋₆alkylNR⁸R⁹;
R⁶ and R⁷ may together form a substituted 5 or 6 membered heterocyclic ring containing one or more heteroatomis selected from N, O or S, which heterocyclic ring may be optionally substituted by A;
R⁸ and R⁹ are independently selected from hydrogen, C₁₋₆alkyl, C₂₋₆alkenyl, C₂₋₆alkynyl, C₀₋₆alylC₃₋₆cycloalkyl, C₀₋₆alkylaryl and C₀₋₆alkylheteroaryl;
R⁸ and R⁹ may together form a 5 or 6 membered heterocyclic ring containing one or more heteroatoms selected from N, O or S, which heterocyclic ring may be optionally substituted by A;
R¹⁰ is hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl or C₁₋₆alkylNR⁸R⁹;
R¹¹ is C₁₋₆alkylNR⁸R⁹ or C₀₋₆alkylheterocycloalkyl;
R¹⁰ and R¹¹ may together form a 5, 6 or 7 membered heterocyclic ring containing one or more heteroatoms selected from N, O or S, which heterocyclic ring-may be optionally substituted by A;
R¹² is a 5, 6 or 7 membered heterocyclic ring containing one or more heteroatoms selected from N, O or S, which heterocyclic ring may be optionally substituted by A;
wherein any C₀₋₆alkyl, C₂₋₆alkenyl, C₂₋₆alkynyl, C₀₋₆alkylC₃₋₆cycloalkyl, C₀₋₆alkylheterocycloalkyl, C₀₋₆alkylaryl, C₀₋₆alkylheteroaryl defied under R⁵ to R¹² may be substituted by one or more A;
A is halo, nitro, CHO, CN, OR⁶, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, CO alkylNR⁶R⁷, OC₁₋₆alkylNR⁶R⁷, CO₂R⁸, CONR⁶R⁷, NR⁶(CO)R⁶, O(CO)R⁶, COR⁶, SR⁶, (SO₂)NR⁶R⁷, (SO)NR⁶R⁷, SO₃R⁶, SO₂R⁶ or SOR⁶, as a free base or a pharmaceutically acceptable salt thereof, with the proviso that the compound is not 4[4-[5-ano-6-(phenylmethyl)pyrazinyl]phenoxy]-ethyl ester butanoic acid.

The present invention further relates to a compound having the formula I 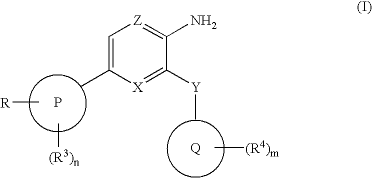
wherein:
Z is N;
Y is CONR⁵, NR⁵ CO, SO₂NR⁵, NR⁵SO₂, CH₂NR⁵, NR⁵CH₂, NR⁵CONR⁵, CH₂CO, COCH₂, CH═CH, OCH₂ or CH₂O;
X is CH or N;
P is phenyl or a 5 or 6 membered heteroaromatic ring containing one or more heteroatoms selected from N, O or S and said phenyl ring or 5 or 6 membered heteroaromatic ring may optionally be fused with a 5 or 6 membered saturated, partially saturated or unsaturated ring containing one or more atoms selected from C, N, O or S;
Q is phenyl or a 5 or 6 membered heteroaromatic ring containing one or more heteroatoms selected from N, O or S of which at least one atom is selected from nitrogen; R is CHO, fluoromethoxy, difluoromethoxy, trifluoromethoxy, C₀₋₆alkyl(SO₂)NR¹R² C₀₋₆alkyl(SO₂)NR¹R², OC₁₋₆alkyl(SO)NR¹R², C₁₋₆alkyl(SO)NR¹R², C₀₋₆alkylNR¹(SO)R², OC₁₋₆alkylNR¹(SO)R², C₀₋₆alkcylNR¹(SO₂)NR¹R², OC₁₋₆alkylNR¹(SO₂)R², C₀₋₆alkyl(SO₂)C₁₋₆alkylNR¹R², OC₀₋₆akyl(SO₂)C₁₋₆alkylNR¹R²,
C₀₋₆alkyl(SO)C₁₋₆alkylNR¹R², OC₁₋₆alkyl(SO)C₁₋₆alkylNR¹R², C₀₋₆alkylSC₁,alkylNR¹R² OC₁₋₆alkylOC₁₋₆alkylNR¹², C₀₋₆alkylCONR¹⁰R¹¹, OC₀₋₆alkylCONR¹R², OC₁₋₆alkylNR¹R², C₀₋₆alkylNR¹⁰(CO)R¹¹, OC₁₋₆alkylNR¹(CO)R², C₀₋₆alkylNR¹¹(CO)R¹⁰, C₀₋₆alkylCOR¹¹, OC₁₋₆alkylCOR¹, C₀₋₆alkylNR¹⁰OR¹¹, C₀₋₆alkylO(CO)R¹¹, C₁₋₆alkylO(CO)R¹¹, (CO)OR¹, O₀₋₆alkylC(NR¹⁰)NR¹⁰R¹¹, C₀₋₆alkylC(NR¹¹)N(R¹⁰), C₀₋₆alkylC(NR¹)NR¹R², C₀₋₆alkylNR¹⁰(CO)OR¹¹, OC₁₋₆alkylNR¹(CO)OR¹, C₀₋₆alkylNR¹¹(CO)OR¹⁰, OC₁₋₆alkylCN, NR¹OR², C₀₋₆alkyl(CO)OR¹, OC₁₋₆alkyl(CO)OR¹, NR¹(CO)NR¹R², NR¹(CO)(CO)R², NR¹(CO)(CO)NR¹R² or SO₃R¹; R¹ and R² are independently selected from hydrogen, C₁₋₆alkyl, C₂₋₆alkenyl C₂₋₆alkynyl, C₀₋₆alkylC₃₋₆cycloalkyl, C₁₋₆alkylR⁶R⁷, C₀₋₆alkylaryl and C₀₋₆alkylheteroaryl, wherein any C₁₋₆alkyl, C₂₋₆alkenlyl, C₂₋₆alkynyl, C₀₋₆alkylC₃₋₆cycloalkyl, C₀₋₆alkylaryl, C₀₋₆alkylheteroaryl may be substituted by one or more A,
R¹ and R² may together from a substituted 5 or 6 membered heterocyclic zing containing one or more heteroatoms selected from N, O or S, and if said heterocyclic ring contains a —NH-moiety that ring nitrogen may be optionally substituted by A;
R³ and R⁴ are independently selected from halo, nitro, CHO, C₀₋₆alkylCN, OC₁₋₆akylCN, C₀₋₆alkylOR⁶, OC₁₋₆alkylOR⁶, fluoromethyl, difluoromethyl, trifluoromethyl, fluoromethoxy, difluoromethoxy, trifluoromethoxy, C₀₋₆alkylNR⁶R⁷, OC₁₋₆alkylNR⁶R⁷, OC₁₋₆alkylOC₁₋₆alkylNR⁶R⁷, NR⁶OR⁷ C₀₋₆alkylCO₂R⁶, OC₁₋₆alkylCO₂R⁶, C₀₋₆alkylCONR⁶R⁷, OC₁₋₆alkylCONR⁶R⁷, OC₁₋₆alkylNR⁶(CO)R⁷, C₀₋₆alkylNR⁶(CO)R⁷, O(CO)NR⁶R⁷, NR⁶(CO)OR⁷₃ NR⁶ (CO)NR⁶R⁷, O(CO)OR⁶, O(CO)R⁶, C₀₋₆alkylCOR⁶, OC₀₋₆alkylCOR⁶, NR⁶(CO)(CO)R⁶, NR⁶(CO)(CO)NR⁶R⁷, SR⁶, C₀₋₆alkyl(SO₂)NR⁶R⁷, OC₀₋₆alkylNR⁶(SO₂)R⁷, OC₀₋₆alkyl(SO₂)NR⁶R⁷, C₀₋₆alkyl(SO)NR⁶R⁷, OC₁₋₆alkyl(SO)NR⁶R⁷, SO₃R⁶, CO 6alkylNR¹(SO₂)NR⁶R⁷, C₀₋₆alkylNR⁶(SO)R⁶R⁷, OC₁₋₆alkylNR⁶(SO)R⁶R⁷, OC₀₋₆alkylSO₂R⁶, C₀₋₆alkylSO₂R⁶, C₀₋₆alkylSOR⁶, C₁₋₆-alkyl,
C₂₋₆alkenyl, C₂₋₆alkynyl, C₀₋₆alkylC₃₋₆cycloalkyl, C₀₋₆alkylaryl and C₀₋₆alkylheteroaryl,
wherein any C₁₋₆alkyl, C₂₋₆alkenyl, C₂₋₆alkynyl, C₀₋₆alkylC₃₋₆cycloalkyl, C₀₋₆alkylaryl and C₀₋₆-alkylheteroaryl may be optionally substituted on any carbon atom by one or more A and if said heteroaryl contains a —NH-moiety that nitrogen may be optionally substituted by
m is 0, 1, 2, 3 or 4;
n is 0,1,2, 3 or 4; —
R⁵ is hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, Ct-₆alkylNR⁶R⁷or C₁₋₆alkylCONR⁶R⁷;
R⁶ and R⁷ are independently selected from hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl and C₁₋₆alkylNR⁸R⁹;
R⁶ and R⁷ may together form a substituted 5 or 6 membered heterocyclic ring containing one or more heteroatoms selected from N, O or S, and if said heterocyclic ring contains a —NH-moiety that ring nitrogen may be optionally substituted by A;
R⁸ and R⁹ are independently selected from hydrogen, C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl;
R⁸ and R⁹ may together form a 5 or 6 membered heterocyclic ring containing one or more heteroatoms selected from N, O or S, and if said heterocyclic ring contains an —NH— moiety that ring nitrogen may be optionally substituted by A;
R¹⁰ is hydrogen, C₁₋₆alkyl, C₂₋₆alkenyl, C₁₋₆alkynyl, C₀₋₆alkylC₃₋₆cycloalkyl, C₀₋₆alkylaryl, C₀₋₆alkylheteroaryl or C₁₋₆alkylNR⁸R⁹;
R¹¹ is C₁₋₆alkylNR⁸R⁹;
R¹⁰ and R¹¹ may together form a 5 or 6 membered heterocyclic ring containing one or more heteroatoms selected from N, O or S, and if said heterocyclic ring contains an —NH— moiety that ring nitrogen may be optionally substituted by A; wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl defined under R¹⁰ to R¹¹ may be substituted by one or more A;
A is halo, nitro, CHO, CN, OR⁶, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR⁶R⁷, OC₁-alkylNR⁶R⁷, CO₂R⁶, CONR⁶R⁷, NR⁶(CO)R⁶, O(CO)R⁶, COR⁶, COR⁶, SR⁶, (SO₂)NR⁶R⁷ (SO)NR⁶R⁷, OR⁶ SOR⁶ or SOR⁶, as a free base or a pharmaceutically acceptable salt thereof.

One aspect of the invention relates to compounds of formula I, wherein:
Z is CH or N;
Y is CONR⁵;
X is CH or N;
P is phenyl or a 5 membered heteroaromatic ring containing one heteroatom selected from O or S;
Q is a 6 membered heteroaromatic ring containing one heteroatom selected from N;
R¹is C₀₋₆alkyl(SO₂)NR¹R², C₆aylCONR¹⁰R¹¹, OC₁₋₆alkylR¹R², C₀₋₆alkyl(CO)OR⁸ or OR¹²;
R¹ and R² are independently selected from hydrogen, C₁₋₆alkyl, (CO)OR⁸, C₀₋₆akylheterocycloalkyl, C₁₋₆alkylNR⁶R⁷ and C₀₋₆alkylheteroaryl, wherein any C₁₋₆alkyl or C₀₋₆alkylheterocycloalkyl may be substituted by one or more A;
R¹ and R² may together form a substituted 5, 6 or 7 membered heterocyclic ring containing one or more heteroatoms selected from N or 9, which heterocyclic ring may be optionally substituted by A;
R³ and R⁴ are independently selected from halo, trifluoromethyl, trifluoromethoxy,
C₀₋₆alkylNR⁶R⁷ and C₁₋₆alkyl;
m is O or l;
n is 0, 1 or 2;
R⁵ is hydrogen;
R⁶ and R⁷ are independently selected from hydrogen, C₁₋₆alkyl and (CO)OR⁸;
R⁶ and R⁷ may together form a substituted 5 or 6 membered heterocyclic ring containing one or more heteroatoms selected from N, which heterocyclic ring may be optionally substituted by A;
R⁸ and R⁹ are independently selected from hydrogen and C₁₋₆alkyl;
R⁸ and R⁹ may together form a 5 or 6 membered heterocyclic ring containing one or more heteroatoms selected from N or O, which heterocyclic ring may be optionally substituted by A;
R¹⁰ is hydrogen or C₁₋₆alkyl;
   R¹¹ is C₁₋₆alkylNR⁸R⁹ or C₀₋₆alkylheterocycloalkyl;
R¹⁰ and R¹¹ may together form a 5, 6 or 7 membered heterocyclic ring containing one or more heteroatoms selected from N, which heterocyclic ring may be optionally substituted
R¹² is a 5, 6 or 7 membered heterocyclic ring containing one or more heteroatoms selected from N, O or S, which heterocyclic ring may be optionally substituted by A;
wherein C₀₋₆alkylheterocycloalkyl defined under R⁵ to R¹ may be substituted by one or more A;
A is OR⁶, C₁₋₆alkyl, C₀₋₆alkylNR⁶R⁷, COR⁶ or CO₂R⁸.

A preferred embodiment of the invention relates to compounds of formula I, wherein Y is CONR⁵.

In one aspect of the invention P is phenyl, furan or thiophene or another 5 or 6 membered heteroaromatic ring containing one or more heteroatoms selected from N, O or S.

In another aspect of the invention preferably Q is pyridine.

In yet another aspect of the invention R is C₀₋₆alkyl(SO₂)NR¹R², (SO₂)NR¹R² or OC₁₋₆alkylNR¹R².

One aspect of the invention relates to compounds wherein R is in the 4 position.

The invention relates to the following compounds;
3-Amino-6-(4[(dimethylamino)sulfonyl]phenyl)-N-pyridin-3-ylpyrazine-2-carboxamide,
3-Amino-6-(3-[(dimethylamino)sulfonyl]phenyl)-N-pyridin-3-ylpyrazine-2-carboxamide,
3-Amino-6-{2-[(dimethylamino)sulfonyl]phenyl}-N-pyridin-3-ylpyrazine-2-carboxamide,
3-Amino-6-[4-(aminosulfonyl)phenyl]-N-pyridin-3-ylpyrazine-2-carboxamide,
2-Amino-5-(4-[(dimethylamino)sulfonyl]phenyl)-N-pyridin-3-ylnicotinamide,
3-Amino-6-(4-([(3-morpholin-4ylpropyl)amino]sulfonyl) phenyl)-N-pyridin-3-ylpyrazine-2-carboxamide and
3-Amino-6-{4-[(4-methylpiperazin-1-yl)sulfonyl]phenyl}-N-pyridin-3-ylpyrazine-2-carboxamide
   as a free base or a pharmaceutically acceptable salt thereof, and
3-Amino-6-{4-[(4-methylpiperazin-1-yl)sulfonyl]phenyl}-N-pyridin-3-ylpyrazine-2-carboxamide hydrochloride.

A further aspect of the invention relates to compounds
3-Amino-6-[4-[2-(4-methyl-1-piperazinyl)ethoxy]phenyl]-N-(3-pyridinyl)-2-pyrazinecarboxamide.
   as a free base or a pharmaceutically acceptable salt thereof, and
3-Amino-6-{4-[(2-methoxy-1-methylethyl)amino]sulfonyl}phenyl)-N-pyridin-3-ylpyrazine-2-carboxamide hydrochloride,
3-Amino-6-{2,5-difluoro-4-[(4-methylpiperazin-1-yl)sulfonyl]phenyl}-N-pyridin-3-ylpyrazine-2-carboxamide hydrochloride and
3-Amino-{3-fluoro-4[(4-methylpiperazin-1-yl)sulfonyl]phenyl}-N-pyridin-3-ylpyrazine-2-carboxamide hydrochloride.

Another aspect of the invention relates to compounds
3-Amino-N-pyridin-3-yl-6-[4-(pyrrolidin-1-ylsulfonyl)phenyl]pyrazine-2-carboxamide,
3-Amino-6-[4-(piperidin-1-ylsulfonyl)phenyl]-N-pyridin-3-ylpyrazine-2-carboxamide,
3-Amino-6-{3-ethyl-4-[(4methylpiperazin-1-yl)sulfonyl]phenyl}N-pyridin-3-ylpyrazine-2-carboxamide,
3-Amino-6-[4-[(4-methylpiperazin-1-yl)sulfonyl]-3-(trifluoromethoxy)phenyl]-N-pyridin-3-ylpyrazine-2-carboxamide,
3-Amino-N-(5-[3-(dimethylamino)propyl]pyridin-3-yl)-6-[4-(piperidin-1-ylsulfonyl)phenyl]pyrazine-2-carboxamide,
3-Amino-N-{5-[3-(dimethylamino)propyl]pyridin-3-yl}-6-[4-(pyrrolidin-1-ylsulfonyl)phenyl]pyrazine-2-carboxamide,
3-Amino-N-{4-[(dimethylamino)methyl]pyridin-3-yl}-6-{4-[(dimethylamino)sulfonyl]phenyl}pyrazine-2-carboxamide,
3-Amino-N-{4-[3-(dimethylamino)propyl]pyridin-3-yl}-6-(4[(dimethylamino)sulfonyl]phenyl)pyrazine-2-carboxamide,
3-Amino-6-[4-(morpholin-4-ylsulfonyl)phenyl]-N-pyridin-3-ylpyrazine-2-carboxamide,
3-Amino-6-[4-ethylpiperazin-1-yl)sulfonyl]phenyl}-N-pyridin-3-ylpyrazine-2carboxamide,
3-Amino-N-pyridin-3-yl-6-(4-{[(2-pyridin-2-ylethyl)amino]sulfonyl}phenyl)pyrazine-2-carboxamide,
3-Amino-6-[4-({[2-(dimethylamino)-1-methylethyl]amino) sulfonyl)phenyl]-N-pyridin-3-ylpyrazine-2-carboxamide,
3-Amino-N-pyridin-3-yl-6-(4-{[(3-pyrrolidin-1-ylpropyl)amino]sulfonyl}phenyl)pyrazine-2-carboxamide,
6-{4-[(4-Acetylpiperazin-1-yl)sulfonyl]phenyl}-3-amino-N-pyridin-3-ylpyrazine-2-carboxamide,
2-Amino-5-{4-[(4-methylpiperazin-1-yl)sulfonyl]phenyl}-N-[4-(pyrrolidin-1-ylmethyl)pyridin-3-yl]nicotinamide,
3-Amino-6-(4-{[[2-(dimethylamino)ethyl](ethyl)amino]carbonyl}phenyl)-N-pyridin-3-ylpyrazine-2-carboxamide,
3-Amino-6-(4-[[[3-(dimethylamino)propyl](methyl)amino]carbonyl]phenyl)-N-pyridin-3-ylpyrazine-2-carboxamide,
3-Amino-6-[4-(([3-(dimethylamino)propyl]amino) carbonyl)phenyl]-N-pyridin-3-ylpyrazine-2-carboxamide,
3-Amino-N-pyridin-3-yl-6-(4-{[(2-pyrrolidin-1-ylethyl)amino]carbonyl}phenyl)pyrazine-2-carboxamide,
3-Amino-N-pyridin-3-yl-6-(4-{[(3-pyrrolidin-1-ylpropyl)amino]carbonyl} phenyl)pyrazine-2-carboxamide,
3-Amino-6-[4-(([2-(dimethylamino)ethyl]amino) carbonyl)phenyl]-N-pyridin-3-ylpyrazine-2-carboxamide,
3-Amino-6-[4-({[2-(dimethylamino)-1-methylethyl]amino}carbonyl)phenyl]-N-pyridin-3ylpyrazine-2-carboxamide,
3-Amino-6-(4-(([3-(4-methylpiperazin-1-yl)propyl]amino} carbonyl)phenyl]-N-pyridin-3-ylpyrazine-2-carboxamide,
3-Amino-6-(4-([(2-piperidin-1-ylethyl)amino]carbonyl} phenyl)-N-pyridin-3-ylpyrazine-2-carboxamide,
3-Amino-N-pyridin-3-yl-6-(4-[(4-pyirolidin-1-ylpiperidin-1-yl)carbonyl]phenyl)pyrazine-2-carboxamide,
4-Amino-4′-[(4-methylpiperazin-1-yl)sulfonyl3-N-pyridin-3-yl-1,1-biphenyl-3-carboxamide,
3-Amino-6-[4-[[[2-(4-morpholinyl)ethyl]amino]carbonyl)phenyl]-N-(3-pyridinyl)-2-*o pyrazinecarboxamide,
tert-Butyl 4-[2-(4-(5-amino-6-[(pyridin-3-ylamino)carbonyl]pyrazin-2-yl) phenoxy)ethyl)piperazine-1 carboxylate,
tert-Butyl 4-[2-(4-(5-amino-6-[(pyridin-3-ylamino)carbonyl]pyrazin-2-yl}-2,5-difluorophenoxy)ethyl]piperazine-1-carboxylate,
-3-Amino-6-{5-[(dimethylamino)sulfonyl] thien-2-yl}-N-pyridin-3-ylpyrazine-2-carboxamide,
tert-Butyl 4-(5-(5-amino-6-[(pyridin-3-ylamino)carbonyl]pyrazin-2-yl-2-furoyl)piperazine-1-carboxylate,
3-Amino-6-(4-([(2-aminoethyl)amino]sulfonyl}-3-(trifluoromethoxy)phenyl]-N-pyridin-3-ylpyrazine-2-carboxamide and
4-{5-Amino-6-[(pyridin-3-ylamino)carbonyl]pyrazin-2-yl) bednzoic acid,
as a free base or a pharmaceutically acceptable salt thereof, and
3-Amino-6-(4-f [[3-(dimethylamiino)propyl] (methyl)amino]sulfonyl) phenyl)-N-pyridin-3-ylpyrazine-2-carboxamide hydrochloride,
3-Amino-6-[4-([3-(4-methylpiperazin-1-yl)propyl]amino) sulfonyl)phenyl)-N-pyridin-3ylpyrazine-2-carboxamide hydrochloride,
3-Amino-N-pyridin-3-yl-6-({[(2-pyrrolidin-1-ylethyl)amino]sulfonyl)phenyl)pyrazine-2-carboxamide hydrochloride,
0.3-Amino-6-[4-(([2-(dimethylamino)propyl]amino}sulfonyl)phenyl]-N-pynzdin-3ylpyrazine-2-carboxamide hydrochloride,
3-Amino-6-(4-({[isopropyl(2-methoxyethyl)amino]sulfonyl phen yl)-N-pyridin-3-ylpyrazine-2-carboxamide hydrochloride,
3-Amino-6-[4-(([2-(diethylamino)ethyl) amino) sulfonyl)phenyl]-N-pyridin-3-ylpyrazine-2-carboxamide hydrochloride,
3-Amino-6-(4 ([[2-(dimethylamino)ethyl](ethyl)amino]sulfonyl) phenyl)-N-pyridin-3-ylpyrazine-2-carboxamide hydrochloride,
3-Amino-6-[4-(([3-(dimethylamno propy]ano) sulfonyl)phenyl)-N-pyridin-3-ylpyrazine-2-carboxamide hydrochloride,
-3-Amino-6-(3-methyl-4-[(4-methylpiperazin-1-yl)sulfonyl]phenyl)-N-pyridin-3-ylpyrazine-2-carboxamide hydrochloride,
3-Amino-6-(2-[(4-methylpiperazin-1-yl)sulfonyl]phenyl)—N-pyridin-3-ylpyrazine-2-carboxamide hydrochloride,
3-Amino-6-{3-[(4-methylpiperazin-1-yl)sulfonyl]phenyl)—N-pyridin-3-ylpyrazine-2-carboxamide hydrochloride,
3-AAmino-6-(2-methyl-4-[(4-methylpiperazin-1-yl)sulfonyl]phetnyl}-N-pyridin-3-ylpyrazine-2-carboxamide hydrochloride,-3-Amino-6-[4-(([2-(dimethylamino)ethyl]amino) sulfonyl)-3-(trifluoromethoxy)phenyl]-N-pyridin-3-ylpyrazine-2-arboxamide hydrochloride,-3-Amino-6-[4 ([[2-(dimethylamino)ethyl](ethyl)amino]sulfonyl}l-3-(trifluoromethoxy)phenyl]-N-pyridin-3-ylpyrazine-2-carboxamide hydrochloride,
-3-Amino-6-[4-[(4-methylpiperazin-1-yl)sulfonyl]-2-(trifluoromethyl)phenyl]-N-pyridin-3-ylpyrazine-2-carboxamide hydrochloride,
3-Arino-6-[4-[2-(dimethylamino)ethoxy]phenyl]-N-(3-pyridinyl)-2-pyraz ne-carboxaride——hydrochloride,
3-Amino-6-[4-[2-(4-morpholinyl)ethoxy]phenyl]-N-(3-pyridinryl)-2-pyrazinecarboxamide hydrochloride,
3-Amino-6-(4-[[[2-(dimethylamino)ethyl]methylamino]carbonyl]phenyl}-N-(3-pyridinyl)-2-pyrazinecarboxamide hydrochloride,
3-Amino-6-(2-fluoro-4-[(4-methylpiperazin-1-yl)sulfonyl]phenyl —N-pyridin-3-ylpyrazine-2-carboxamide hydrochloride,
-3-Amino-6-{5-fluoro-2-methyl][(4methylpiperazin-1-yl)sulfonyl]phenyl)—N-pyridin-3-ylpyrazine-2-carboxamide hydrochloride,
3-Amino-6-(2,5-dimethyl4[(4-methylpiperazin-1-yl)sulfonyl]phenyl}-N-pyridin-3-ylpyrazine-2-carboxamide hydrochloride,
3-Amino-6-[4-(2-piperidin-1-ylethoxy)phenyl]-N-pyridin-3-ylpyrazine-2-carboxamide hydrochloride,
3-Amino-6-[4-(2-pyrrolidin-1-ylethoxy)phenyl]-N-pyridin-3-yl-pyrazine-2-carboxamide hydrochloride,
3-Amino-6-[2,5-difluoro-4-(2-morpholin-4-ylethoxy)phenyl]-N-pyridin-3-ylpyrazine-2-carboxamide hydrochloride,
3-Amino-6-4-[(4-methylpiperazin-1-yl)carbonyl]phenyl)-N-pyridin-3-ylpyrazine-2-carboxamide hydrochloride,
3-Amino-6-[2,5-difluoro4-(2-pyrrolidin-1-ylethoxy)phenyl]-N-pyridin-3-ylpyrazine-2-carboxamide hydrochloride,
3-Amino-6-1{2,6-dimethyl-4-[2-(4methylpiperazin-1-yl)ethoxy]phenyl)-N-pyridin-3-ylpyrazine-2-carboxamide hydrochloride,
3-Amino-6-.{2-methyl-4[2-(4-methylpiperazin-1-yl)ethoxy]phenyl)-N-pyridin-3ylpyrazine-2-carboxamide hydrochloride,
2-Amino-5-(4-[(4-methylpiperazin-1-yl)sulfonyl]phenyl}-N-pyridin-3-ylnicotinamide hydrochloride,
3-Amino-6-(4-[(4-methylpiperazin-1-yl)sulfonyl]phenyl 3-N-[4-(pyrrolidin-1-ylmethyl)pyridin-3-yllpyrazine-2-carboxamide hydrochloride,
3-Amino-6-[2,5-difluoro4(pyrrolidin-1-ylsulfonyl)phenyl)-N-[4-(2-pyrrolidin-1-ylethyl)pyridin-3-yl]pyrazine-2-arboxamide hydrochloride,
3-Amino-6-[2,5-difluoro-4-(pyrrolidin-1-ylsulfonyl)phenyl]-N-[5-(3-pyrrolidin-1-ylpropyl)pyridin-3-yl]pyrazine-2-carboxamide hydrochloride,
-3-Amino-6[2,5-difluoro4-(piperidin-1-ylsulfonyl)phenyl]-N-[5-(3-pyrrolidin-1-ylpropyl)pyridin-3-yl]pyrazine-2-carboxamide hydrochloride,
3-Amino-6-[4(piperidin-1-ylsulfonyl)phenyl]-N-[5-(3-pyrrolidin-1-ylpropyl)pyridin-3-yl]pyrazine-2-carboxamide hydrochloride,
3-Amino-N-[5-(3-pyrrolidin-1-ylpropyl)pyridin-3-yl]-6-(4-(pyrrolidin-1ylsulfonyl)phenyl]pyrazine-2-carboxamide hydrochloride,
3-Amino-N-[4-(2-pyrrolidin-1-ylethyl)pyridin-3-yl]-6-[4-(pyrrolidin-1-ylsulfonyl)phenyl]pyrazine-2-carboxamide hydrochloride,
3-Amino-N-([(3-pyrrolidin-1-ylprbpyl)pyridin-3-yl]-6-[4(pyrrolidin-1-*ylsulfonyl)phenyl]pyrazine-2-carboxamide hydrochloride,
3-Amino-N-(4-(pyrrolidin-1-ylmethyl)pyridin-3-yl]-6-[4-(pyrrolidin-1-ylsulfonyl)phenyl]pyrazine-2-carboxamide hydrochloride,
3-Amino-N-(4-[(dimethylamino)methy)]pyridin-3-yl}-6-[4-(pyrrolidin-1ylsulfonyl)phenyl]pyrazine-2-carboxamide hydrochloride,
3-Amino-N-{4-[(dimethylamino)methyl)pyridin-3-yl)-6-[4-(piperidin-1-ylsulfonyl)phenyl]pyrazine-2-carboxamide hydrochloride,
3-Amino-6-(3-ethyl-4-[(4-methylpiperazin-1-yl)sulfonyl]phenyl)-N-pyridin-3-ylpyrazine-2-carboxamide hydrochloride,
3-Am no-6-[4-[(4-methylpiperazin-1-yl)sulfonyl]-3-(trifluoromethoxy)phenyl)-N-pyridin-3-ylpyrazine-2-carboxamide.hydrchloride,
3-Amino-6-[4-([(2-aminoethyl)amino]sulfonyl)-3-(trifluoromethoxy)phenyl]-N-pyridin-3-ylpyrazine-2-carboxamide hydrochloride,
4-Amino-4′-((4-methylpiperazin-1-yl)sulfonyl]-N-pyridin-3-yl-1,1′-biphenyl-3-carboxamide hydrochloride,
2-Amino-5-[4-[(4-methylpiperazin-1-yl)sulfonyl]phenyl)-N-[4-(pyrrolidin-1ylmethyl)pyridin-3-yllnicotinaamide hydrochloride 3-Amino-N-pyridin-3-yl-6-[4-(pyrrolidin-1-ylsulfonyl)phenyllpyrazine-2-carboxamide hydrochloride,
3-Amino-6-[4-(piperidin-1-ylsulfonyl)phenyl]-N-pyridin-3-ylpyrazine-2-carboxamide hydrochloride,
*3-Amino-6-[4-(piperazin-17ylsulfonyl)phenyl]-N-pyridin-3-ylpyrazine-2-carboxamide hydrochloride,
-3-Amino-6-[4-(2-piperazin-1 ylethoxy)phenyl]-N-pyridin-3-ylpyrazine-2-carboxamide hydrochloride,
3-Amino-6-[2,5-difluoro-4(2-piperazin-1-ylethoxy)phenyl]-N-pyridin-3-ylpyrazine-2carboxamide hydrochloride,
3-Amino-6-[5-(piperatin-l ylcarbonyl)-2-furyl]-N-pyridin-3-ylpyrazine-2-carboxamide hydrochloride and 3-Amino-N-(5-[3-(dimethylamino)propyl)pyridin-3-yl}-6-[4-(piperidin-1-ylsulfonyl)phenyl]pyrazine-2-carboxamide hydrochloride. Yet another aspect of the invention relates to compounds tert-Butyl 4-[(4-(5-amino-6-[(pyridin-3-ylamino)carbonyllpyrazin-2-yl} phenyl)sulfonyl]piperazine-1-carboxylate,
3-Amino-6-(4-{[methyl(1-methylpyrrolidin-3-yl)amino]sulfonyl) phenyl)-N-pyridin-3-ylpyrazine-2-carboxamide,
3-Amino-6-(4-{[methyl(1-methylpiperidin-4-yl)amino]sulfonyl}phenyl)-N-pyridin-3-ylpyrazine-2-carboxamide,
3-Amino-6-(4-([3-(dimethylamino)pyrrolidin-1-ylsulfonyl) phenyl)-N-pyridin-3-ylpyrazine-2-carboxamide,
3-Amino-6-(4-[(4-methyl-1,4-diazepan-1-yl)carbonyl]phenyl)—N-pyridin-3-ylpyrazine-2-carboxamide,
3-Amino-6-(4-((methyl(1-methylpyrrolidin-3-yl)amino]carbonyl phenyl)-N-pyridin-3-ylpyrazine-2-carboxamide,
3-Amino-&(4 ([3-(dimethylamino)pyrrolidin-1 yl]carbonyl] phenyl)-N-pyridin-3-ylpyrazine-2-carboxamide,
3-Amino-6-[4-([(1-ethylpyrrolidin-2-yl)methyl]amino) carbonyl)phenyl]-N-pyridin-3-ylpyrazine-2-carboxamide,
-3-Amino-6-(4-{[methyl(1-methylpiperidin-4-yl)amino]carbonyl)phenyl)-N-pyridin-3-ylpyrazine-2-carboxamide,
3-Amino-6-(4-{[(1-ethylpiperidin-3-yl)amino]carbonyl) phenyl)-N-pyridin-3-ylpyrazine-2-carboxamide,
3-Amino-6-[4(([2-(1-methylpyrrolidin-2-yl]ethyl]amino) carbonyl)phenyl]-N-pyridin-3-ylpyrazine-2-carboxamide,
tert-Butyl 2-{(4-{5-amino-6-[(pyridin-3-ylamino)carbonyl]pyrazin-2-yl}phenyl)sulfonyl]-(tert-butoxycarbonyl)amino)ethylcarbamate and
3-Amino-6-14-[(1-methyl-3-pyrrolidinyl)oxy]phenyl]-N-(3-pyridinyl)-2-pyrazinecarboxamide,:
   as a free base or a pharmaceutically acceptable salt thereof, and 3-Amino-6-{-[(4-methyl-1,4-diazepati-1-yl)sulfonyl]phenyl)-N-pyridin-3-ylpyrazine-2-carboxamide hydrochloride and 3-Amino-6-[4-([(1-ethylpyrrolidin-2-yl)methyl]amino)sulfonyl)phenyl]-N-pyridin-3-ylpyrazine-2-carboxamide hydrochloride.

Listed below are definitions of various terms used in the specification and claims to describe the present invention.

In this specification the term “alkyl” includes both straight and branched chain alkyl groups. The term C₁₋₆alkyl having 1 to 6 carbon atoms and may be methyl, ethyl, n-propyl, i-propyl, n-butyl, i-butyl, s-butyl, t-butyl, n-pentyl, i-pentyl, t-pentyl, neo-pentyl, n-hexyl or i-hexyl. The term C₁₋₃alkyl having 1 to 3 carbon atoms and may be methyl, ethyl, n-propyl or i-propyl. The term C₁₋₂alkyl having 1 to 2 carbon atoms and may be methyl or ethyl.

A similar convention applies to other radicals, for example “C₀₋₆alkylaryl” includes 1-phenylethyl and. 2-phenylethyl.

In the case where a subscript is the integer 0 (zero) the group to which the subscript refers to indicates that the group may be absent, i.e. there is a direct bond between the groups.

The term “cycloalkyl” refers to an optionally substituted, saturated cyclic hydrocarbon ring system. The term “C₃₋₆cycloalkyl” may be cyclopropyl, cyclobutyl, cyclopentyl or 0.20 cyclohexyl.

The term “alkenyl” refers to a straight or branched chain alkenyl group. The term C₂₋₆alkenyl having 2 to 6 carbon atoms and one double bond, and may be vinyl, allyl, propenyl, i-propenyl, butenyl, i-butenyl, crotyl, pentenyl, i-pentenyl or hexenyl. The term C₂₋₃alkenyl having 2 to 3 carbon atoms and one or two double bond, and may be vinyl, allyl, propenyl or i-propenyl.

The term “alkynyl” refers to a straight or branched chain alkenyl group C₂₋₆alkynyl having 2 to 6 carbon atoms and one triple bond, and may be ethynyl, propargyl, butynyl, i-butynyl, pentynyl, i-pentynyl or hexynyl. The term C₂₋₃alkynyl having 2 to 3 carbon atoms and one triple bond, and may be ethynyl or propargyl.

The term “halo” refers to fluoro, chloro, bromo and iodo.

The term “aryl” refers to an optionally substituted monocyclic or bicyclic hydrocarbon ring system containing at least one unsaturated aromatic ring. The “aryl” may be fused with a C₅₋₇cycloalkyl ring to form a bicyclic hydrocarbon ring system. Examples and suitable values of the term “aryl” are phenyl, naphthyl, indanyl or tetralinyl.

The term “heteroaryl” and “5 or 6 membered heteroaromatic ring” containing one or more heteroatoms selected from N, O and S may be furyl, imidazolyl, isoxazolyl, isothiazolyl, oxazolyl, pyrazinyl, pyrazolyl, pyridazinyl, pyridyl, pyrimidyl, pyrrolyl, thiazolyl or thienyl.

The term “heterocycloalkyl” and “heterocyclic ring containing one or more heteroatoms selected from N, O or S may optionally contain a carbonyl function and is preferably a 5, 6 or 7 membered heterocyclic, ring and may be imidazolidinyl, imidazolinyl, morpholinyl, piperazinyl, piperidinyl, piperidonyl, pyrazolidinyl, pyrazolinyl, pyrrolidinyl, pyrrolinyl, 1-methyl-1′,4-diazepane, tetrahydropyranyl, thiomorpholinyl. In the case where the heterocyclic ring contains a heteroatom selected from S this includes optionally SO and SO₂.

It is to be understood that when m is greater than one, R⁴ groups may be the same or different. Similarly, when m is greater, than one the R³ groups may be the same or different.

The term “hydrochloride” includes monohydrochloride, dihydrochloride, trihydrochloride and tetrahydrochloride salts.

A suitable pharmaceutically acceptable salt of the compound of the invention is, for example, an acid-addition salt, which is sufficiently basic, for example an inorganic or organic acid. In addition a suitable pharmaceutically acceptable salt-of the compounds of the invention which is sufficiently acidic is an alkali metal salt, an alkaline earth metal salt or a salt with an organic base which affords a physiologically-acceptable cation.

Some compounds of formula I may have chiral centres and/or geometric isomeric centres (E- and Z-isomers), and it is to be understood that the invention encompasses all such optical, diastereoisomers and geometric isomers.

The invention relates to any and all tautomeric forms of the compounds of formula I.

The invention also relates to a compound of formula XI 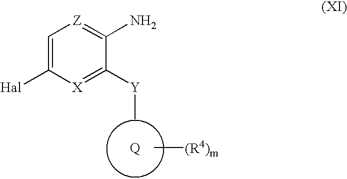
wherein Y, X, Z, Q, R⁴, R⁵, R⁶, R⁷, R⁸, R⁹, A and m are defined as in formula I.

The invention further relates to a compound of formula XIII 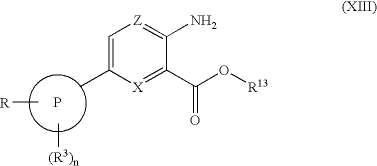
wherein X, Z, P, R, R¹, R², R³, R⁶, R⁷, R⁸, R⁹, R¹⁰, R¹¹, R¹², A and n are defined as in formula I and R¹³ is hydrogen or C₁₋₆alkyl.

One aspect of the invention relates to a compound of formula XV 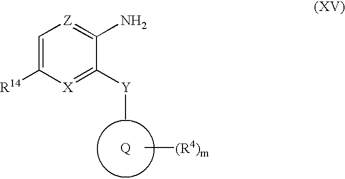
wherein Y, Z, X, Q, R⁴, R⁵, R⁶, R⁷, R⁸, R⁹, A and m are defined as in formula I and R¹⁴ is diethylboronate, 1,3,2-dioxaborolane, 1,3,2-dioxaborinane or 1,3,2-benzodioxaborole.

Another aspect of the invention relates to a compound of formula XVI 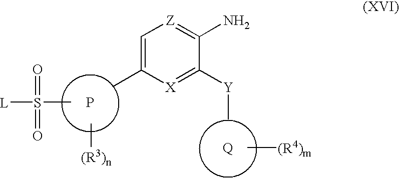
wherein Y, Z, X, P, Q, R³, R⁴, R⁵, R⁶, R⁷, R⁸, R⁹, A, m and n are defined as in formula I and L is a leaving group.

A further aspect of the-invention relates to the following compounds, which may be used as intermediates for the preparation of a compound of formula I;
3-Amino-6-bromo-N-pyridin-3-ylpyrazine-2-carboxamide,
N,N-Dimethyl-4-(4,4,5,5-tetramethyl-[1,3,2]-dioxaborolan-2-yl)benzenesulfonamide;
N,N-Dimethyl-3-(4,4,5,5-tetramethyl-1,3,2-dioxaborolan-2-yl)benzenesulfonamide,
N,N-Dimethyl-2-(4,4,5,5-tetramethyl-1,3,2-dioxaborolan-2-yl)benzenesulfonamide,
2-Amino-5-bromo-N-pyridin-3-ylnicotinamiide,
4-(4,4,5,5-Tetramethyl-1,3,2-dioxaborolan-2-yl)benzenesulfonamide,
3-Amino-6-[4-(}[2-(dimethylamino)ethyl]amino}sulfonyl)phenyl]-N-pyridin-3-ylpyrazine-2-carboxamide,
4-{[(3-Morpholin-4-ylpropyl)amino]sulfonyl}phenylboronic acid,
4-[(4-Methylpiperazin-1-yl)sulfonyl]phenylboronic acid,
4-Bromo-N-[2-(dimethylamino)ethyl]benzenesulfonamide,
4-Bromo-N-(3-morpholin-4-ylpropyl)benzenesulfonamide,
1-[(4-Bromo-2,5-difluorophen-yl)sulfonyl]-4-methylpiperazine,
1-[(4-Bromo-2-ethylphenyl)sulfonyl]-4-methylpiperazine,
1-([4-Bromo-2-(trifluoromethoxy)phenyl)sulfonyl]-4-methylpiperazine,
1-[(Bromo-2-fluorophenyl)sulfonyl]-4-methylpiperazine,
1-[(4-Bromo-2-methylphenyl)sulfonyl]-4-methylpiperazine,
1-[(2-Bromophenyl)sulfonyl]-4-methylpiperazine,
1-[(3-Bromophenyl)sulfonyl]4-methylpiperazine,
4-Bromo-N-[2-(dimethylamino)ethyl]-2-(trifluoromethoxy)benzenesulfonamide,
4-Bromo-N-[2-(dimethylamino)ethyl]-N-ethyl-2-(trifluoromethoxy)benzenesulfonamide,
N-(2-Aminoethyl)-4-bromo-2-(trifluoromethoxy)benzenesulfonamide,
tert-Butyl 2-({[4-bromo-2-(trifluoromethoxy)phenyl]sulfonyl},
(tert-butoxycarbonyl)amino)ethylcarbamate,
4-Bromo-N-methyl-N-(1-methylpyrrolidin-3-yl)benzenesulfonamide,
4Bromo-N-[2-(dimethylamino)-1-methylethyl]benzenesulfonamide,
4-Bromo-N-(3-pyrrolidin-1-ylpropyl)benzenesulfonamide,
1-Acetyl-4-[(4-bromophenyl)sulfonyl]piperazine,
4-Bromo-N-methyl-N-(1-methylpiperidin-4-yl)benzenesulfonamide,
4-Bromo-N-[3-(dimethylamino)propyl]-N-methylbenzenesulfdnam-ide,
4-Bromo-N-[2-(dimethylamino)ethyl]-N-methylbenzenesulfonamide,
4-Bromo-N-[3-(4-methylpiperazin-1-yl)propyl]benzenesulfonamide,
1-[(4-Bromophenyl)sulfonyl]4-4ethylpiperazine,
4-Bromo-N-(2-pyrrolidin-1-ylethyl)benzenesulfonamide,
1-[(4-Bromophenyl)sulfonyl]-4-methyl-1,4-diazepane,
4-Bromo-N-[2-(dimethylamino)propyl]benzenesulfonamide,
4-Bromo-N-[(1-ethylpyrrolidin-2-yl)methyl]benzenesulfonamide,
4-Bromo-N-[2-(diethylamino)ethyl]benzene sulfonamide,
4-Bromo-N-(2-pyridin-2-ylethyl)benzenesulfonamide,
4-Bromo-N-[3-(dimethylamino)propyl]benzenesulfonamide,
1-[(4-Bromophenyl)sulfonyl]-N,N-dimethylpyrrolidin-3-amine,
4-[(4-Bromophenyl)sulfonyl]morpholine,
4-Bromo-N-isopropyl-N-(2-methoxyethyl)benzenesulfonamide,
4-Bromo-N-(2-methoxy-1-methylethyl)benzenesulfonamide,
4-Bromo-N-[2-(dimethylamino)ethyl]benzamide,
4-Bromo-N-[2-(dimethylamino)ethyl]-N-methylbenzamide,
N-[2-Fluoro-4-[(4-methyl-1-piperazinyl)sulfonyl]phenyl]acetamide,
2-Methyl-4-[(4-methylpiperazin-1-yl)sulfonyl]aniline,
1-[(4-Bromo-3-methylphenyl)sulfonyl]4-methylpiperazine,
2-Fluoro-4-[(4-methyl-1-piperazinyl)sulfonyl]benzenamine,
1-[(4-Bromo-3-fluorophenyl)sulfonyl]-methylpiperzine,
-4-[(4Methylpiperazin-1-yl)sulfonyl]-2-(trifluoromethyl)aniline,
1-([4-Bromo-3-(trifluoromethyl)phenyl]sulfonyl)-4-methylpiperazine,
-[(4-Bromo-2-floro-5-methylphenyl)sulfonyl]-4-methylpiperazine,
1-[(4-Bromo-2,5-dimethylphenyl)sulfonyl]-4-methylpiperazine,
1-[(4-Bromophenyl)sulfonyl]piperidine,
1-[(4-Bromophenyl)sulfonyl]pyrrolidine,
1-[(4-Bromo-2,5-difluorophenyl)sulfonyl]piperidine,
1-[(4-Bromo-2,5-difluorophenyl)sulfonyl]pyrrolidine,
tert-Butyl 4[(4-bromophenyl)sulfonyl]piperazine-1-carboxylate,
1-(4-Bromobenzoyl)methylpiperazine,
3-(4-Bromophenoxy)-1-methylpyrrolidine,
tert-Butyl 4-[2-(4-bromophenoxy)ethyl]piperazine-1-carboxylate,
tert-Butyl 4-[2-(4-bromo-2,5-difluorophenoxy)ethyl]piperazine-1-carboxylate,
4-[2-(4Bromo-2,5-difluorophenoxy)ethyl]morpholine,
1-[2-(4-Bromo-3,5-dimethylphenoxy)ethyl])methylpiperazine,
1-[2-(4Bromo-3-methylphenoxy)ethyl]4-methylpiperazine,
1-[2-(4Bromo-2,5-difluorophenoxy)ethyl]pyrrolidine,
5-Bromo-N,N-dimethylthiophene-2-sulfonamide,
tert-Butyl 4-(5-bromo-2-fluroyl)piperazine-1-carboxylate,
3-Ethyl-4-[(4-methylpiperazin-1-yl)sulfonyl)phenylboronic acid,
4-[(4-Methylpiperazin-1-yl)sulfonyl]-3-(trifluoromethoxy)phenylboronic acid,
4-([4-(tert-Butoxycarbonyl)piperazin-1-yl]sulfonyl)phenylboronic acid,
2,5-Difluoro-4-(piperidin-1-ylsulfonyl)phenylboronic acid,
2,5-Difluoro-4(pyrrolidin-1-ylsulfonyl)phenylboronic acid,
4-(Pyrrolidin-1-ylsulfonyl)phenylboronic acid,
4-(Piperidin-1-ylsulfonyl)phenylboronic acid,
4-[(Dimethylamino)sulfonyl]phenylboronic acid,
4-((Methyl(−1-methylpyrrolidin-3-yl)amino)sulfonyl)phenylboronic acid,
4-((4-Acetylpiperazin-1-yl)sulfonyl)phenylboronic acid,
4-(((2-Dimethylamino)ethyl)(ethyl)amino)sulfonyl)phenylboronic acid,
4-((3-Dimethylamino)pyrrolidin-1-yl)sulfonyl)phenylboronic acid,
4-(((2-Dimethylamino)-1-methylethyl)amino)sulfonyl)phenylboronic acid,
4-((3-Pyrrolidin-1-ylpropyl)amino)sulfonyl)phenylboronic acid,
4-((Methyl-(1-methylpiperidin-4-yl)amino)sulfonyl)phenylboronic acid,
4(((Dimethylamino)propyl)(methyl)amino)sulfonyl)phenylboronic acid,
4-(Morphoin-4-ylsulfonyl)phenylboronic acid,
4-(((3-(4-Methylpiperazin-1-yl)propyl)amino)sulfonyl)phenylboronic acid,
4((4-Ethylpiperazin-1-yl)sulfonyl)phenylboronic acid,
4-((2-Pyrrolidin-1-ylethyl)amino)sulfonyl)phenylboronic acid,
4-((4-Methyl-1,4-diazepan-1-yl)sulfonyl)phenylboronic acid,
4-(((2-Dimethylamino)propyl)amino)sulfonyl)phenylboronic acid,
4-((Isopropyl-(2-methoxyethyl)amino)sulfonyl)phenylboronic acid,
4-((((1-Ethylpyrrolidin-2-yl)amino)sulfonyl)phenylboronic acid,
4-(((2-Diethylamino)ethyl)amino)sulfonyl)phenylboronic acid,
4-(((2-Pyridin-2-ylethyl)amino)sulfonyl)phenylboronic acid,
4-(((2-Methoxy-1-methylethyl)amino)sulfonyl)phenylboronic acid,
4-(((3-Dimethylamino)propyl)amino)sulfonyl)phenylboronic acid,
tert-Butyl 4-[(dimethylamino)methyl]pyridin-3-ylcarbamate,
4-[(Dimethylamino)methyl]pyridin-3-amine,
4-(Pyrrolidin-1-ylmethyl)pyridin-3-amine,
4-(2-Pyrrolidin-1-ylethyl)pyridin-3-amine,
4(3-Pyrrolidin-1-ylpropyl)pyridin-3-amine,
tert-Butyl 4-(pyrrolidin-1-ylmethyl)pyridin-3-ylcarbamate,
tert-Butyl 4-(2-pyrrolidin-1-ylethyl)pyridin-3-ylcarbamate,
tert-Butyl 4-(2-hydroxyethyl)pyridin-3-ylcarbamate,
tert-Butyl 4-(3-pyrrolidin-1-ylpropyl)pyridin-3-ylcarbamate,
tert-Butyl 4-(3-pyrrolidin-1-ylprop-1-yl)pyridin-3-ylcarbamate,
tert-Butyl 5-(3-pyrrolidin-1-ylprop-1-ynyl)pyridin-3-ylcarbamate,
tert-butyl 4-[3-(dimethylamino)prop-1-ynyl]pyridin-3-ylcarbamate,
4-(3-Dimethylaminopropyl)pyridin-3-ylamine,
5-(3-Pyrrolidin-1 ylpropyl)pyridin-3-armine,
tert-Butyl 4-(3-hydroxyprop-1-ynyl)pyridin-3-ylcarbamnate,
tert-Butyl 0.5-(3-hydroxyprop-1-ynyl)pyridin-3-ylcarbamate,
tert-Butyl 5-[3-(dimethylamino)prop-1-ynyl]pyridin-3-ylcarbamate,
tert-Butyl 5-bromopyridin-3-ylcarbamate,
tert-Butyl 5-[3-(dimethylamino)propyl]pyridin-3-ylcarbamate,
5-[3-(Dimethylamino)propyl]pyridin-3-amine,
2-Amino-5-bromo-N-(3-pyridinyl)benzamide,
2-Amino-5-bromo-N-[4-(pyrrolidin-1-ylmethyl)pyridin-3-yl]nicotinamide,
3-Amino-6-bromo-N-[4-(pyrrolidin-1-ylmethyl)pyridin-3-yl]pyrazine-2-carboxamide,
3-Amino-6-bromo-N-[4-(2-pyrrolidin-1-ylethyl)pyridin-3-yl]pyrazine-2-carboxamide,
3-Amino-6-bromo-N-{4-[(dimethylamino)methyl]pyridin-3-yl}pyrazine-2-carboxamide,
3-Amino-6-bromo-N-{5-[3-(dimethylamino)propyl]pyridin-3-yl}pyrazine-2-carboxamide,
3-Amino-6-bromo-N-[5-(3-pyrrolidin-1-ylpropyl)pyridin-3-yl]pyrazine-2-carboxamide,
Methyl 3-amino-6-{4-[(dimethylamino)sulfonyl]phenyl}pyrazine-2-arboxylate,
3-Amino-6-{4-[(dimethylamino)sulfonyl]phenyl}pyrazine-2-carboxylic acid,
tert-Butyl 4-formylpyridin-3-ylcarbamate,
3-Amino-6-[4-(pyrrolidin-1-ylsulfonyl)phenyl]pyrazine-2-carboxylic acid and
Methyl 3-amino-6-[4-(pyrrolidin-1-ylsulfonyl)phenyl]pyrazine-2-carboxylate.
   Methods-of Preparation

Another aspect of the present invention provides a process for preparing a compound of formula I as a free base or a pharmaceutically acceptable salt thereof. Throughout the following description of such processes it is understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis” T. W. Green, P. G. M. Wuts, Wiley-Interscience, New York, 1999.

Methods of Preparation of the Intermediates.

The process for the preparation of the intermediates, wherein Y, X, Z, P, Q, R¹, R¹, R², R³, R⁴, R⁵, R⁶, R⁷, R⁸, R⁹, R¹⁰, R¹¹, R¹², A, m and n are, unless specified otherwise, defined as in formula I, comprises of:

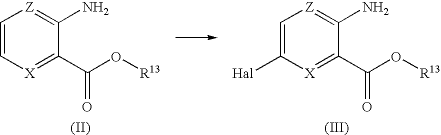

(i) halogenation of a compound of formula II wherein X and Z are N or CH, R¹³ is hydrogen, C₁₋₆alkyl or when R¹³ is hydrogen in the form of a salt such as a sodium salt, to obtain a compound of formula III, may be carried out using a suitable halogenating reagent such as iodine, bromine, chlorine, halide salts such as ICI, BrCl or HOCl or other suitable halogenation reagents such as N-bromosuccinimide or phosphorous tribromide. The reaction may be catalysed by metals or acids such as Fe, Cu-salts, acetic acid or sulfuric acid or aided by oxidising agents such as nitric acid, hydrogen peroxide or sulfur trioxide. The reaction may be carried out in a suitable solvent such as water, acetic acid or chloroform at a temperature in the range of −700C to +100° C.

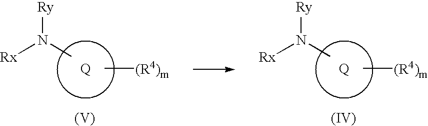

(ii) reacting a compound of formula V, wherein Q is a pyridine ring, R⁴ is hydrogen (when m=0), bromine or iodide, m is 1 and wherein at least one of Rx or Ry is a suitable protecting group CO₂R⁸ to form a carbamate such as tert-butyl carbamate and the other of the Rx or Ry (in the case of one protecting group) is hydrogen, to obtain a compound of s5 formula IV, wherein Q is a pyridine ring, R⁴ is C₁₋₆alkylNR⁶R⁷ and m is 1, may be carried out by,

a) a reaction with butyllithium in a suitable solvent such as tetrahydrofuran or hexane followed by the addition of a suitable reagent such as ethylene oxide followed by the activation of the formed alcohol by the formation of the mesylate or the tosylate with a suitable reagent such as methansulfonyl chloride or para-toluensulfonyl chloride in a suitable solvent such as methylene chloride or tetrahydrofuran with or without a suitable base such as potassium carbonate or a trialkyl amine such as triethyl amine and at a suitable reaction temperature range between 0° C. and +100° C., followed by the addition of the appropriate amine HNR⁶R⁷ at a reaction temperature range between 0° C. and +100° C.; or,

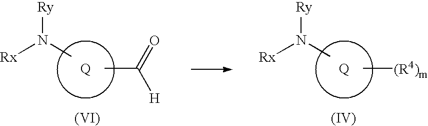

b) reacting a compond of formula VI, wherein Q is as defined above and wherein at least one of Rx or Ry is a suitable protecting group CO₂R⁸, to form a carbamate such as tert-butyl carbamate and the other of the Rx or Ry (in the case of one protecting group) is hydrogen, with the appropriate amine HNR⁶R⁷ in the presence of a suitable reducing reagent such as sodium cyanoborohydride or sodium triacetoxyborohydride in a suitable solvent such as methylene chloride, 1,2-dichloroethane and at a reaction temperature range between 0° C. and +80° c; or,

c) reacting a compound of formula V, wherein Q is a pyridine ring, R⁴ is bromine or iodide and m is 1, 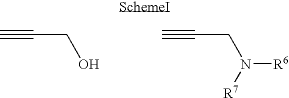
in a palladium catalysed reaction using a suitable palladium reagent such as palladium tetrakistriphenylphosphine in the prescence of a copper(I) halide such as CuI and a suitable base such as potassium carbonate or a trialkyl amine such as triethyl amine, and a compound described in Scheme I. The reaction may be performed in a solvent such as dioxane, tetrahydrofaran, toluene or acetonitrile at temperatures between +25° C. and +100° C.

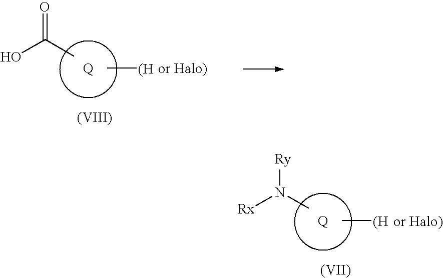

(iii) conversion of a compound of formula VIII, wherein Q is as defined above, to obtain a compound of formula VII, wherein Rx and Ry are hydrogen or at least one of Rx or Ry is a suitable protecting group CO₂R⁸, to form a carbamate such as tert-butyl carbamate and the other of the Rx or Ry (in the case of one protecting group) is hydrogen, may be carried out by,

a) a reaction of a compound of formula VII with a suitable reagent such as diphenylphosphorylazide in tert-butanol and at a temperature interval between +25° C. and +100° C.; or,

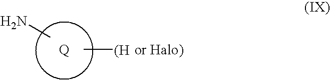

b) by treatment of a compound of formula LX with a suitable tert-butyl carbamate formation reagent such as di-tert-butyl dicarbonate in a suitable solvent such as methylene chloride or chloroform and at a suitable temperature interval between 0° C. and +60° C.

(iv) hydrolysis of a compound of formula IV, to obtain a compound of formula X, 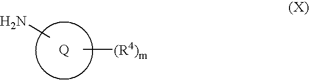
wherein Q is as defined above, R⁴ is C₁akylNR⁶R⁷ and m is 1, may be carried out by treating a compound of formula IV under acidic conditions using suitable acids such as hydrochloric acid or trifluoroacetic acid neat or in an appropriate solvent such as methanol, acetonitrile, methylene chloride or tetrahydrofuran and at a temperature interval between 0° C. and +80° C.

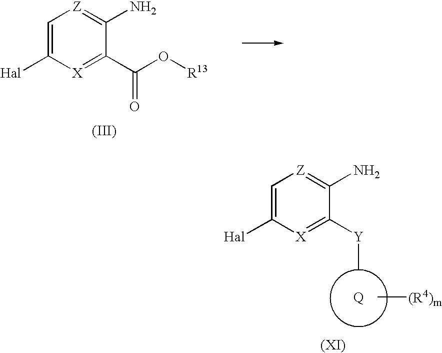

(v) amidation of a compound of formula m, wherein X and Z are N or CH, R¹³ is C₁₋₆alkyl to obtain a compound of formula XI, wherein Y is CONR⁵ may be carried out by treating a compound of formula III with the appropriate amine such as a compound of formula X or 3-aminopyridine. The reaction may be performed neat or using a suitable solvent such as N,N-dimethylformrnamide, methylene chloride or ethyl acetate at a temperature ranging from −25° C. to +150° C. The reaction may be aided by using a base such as potassium carbonate, trietylamine or 1,8-diazabicyclo[5.4.0]undec-7-ene or an acid such as trimethylaluminum or p-toulenesulfonic acid.

(vi) amidation of a compound of formula III, wherein R¹ is hydrogen, to obtain a compound of formula XI, wherein Y is CONR⁵ and R⁴ is a substituent that is not susceptible to certain coupling agents, may be performed by activation of a compound of formula II by treating the compound with coupling reagents such as 1-[3-(dimethylamino)propyl]-3-ethylcarbodiimide hydrochloride and 1-hydroxybenzotriazole hydrate, 1,3-dicyclohexylcarbodiimide and 1-hydroxybenzotriazole hydrate, 1,1′-carbonyldiimidazole or. O-(7-azabenzotriazol-1-yl)-N,N,N′N′-tetramethyluronium hexafluorophosphate or using an acyl halide reagent such as cyanuric chloride, oxalyl chloride, thionyl chloride or bromotrispyrrolidinophosphonium hexafluorophosphate, followed by treatment with the appropriate amine such as a compound of formula X or 3-aminopyridine.

(vii) amidation of a compound of formula II, wherein R¹³ is hydrogen or C₁₋₆alkyl, to obtain a compound of formula XI, may be carried out by amidation conditions described in (v) and (vi) above to obtain a compound of formula X, wherein Y is CONR⁵ and R⁴ is a substituent that is not susceptible to certain coupling agents; 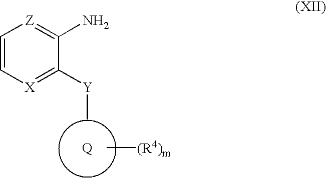
followed by,
halogenation of a compound of formula XII with a halogenating reagent as described in (i) above to obtain a compound of formula XI.

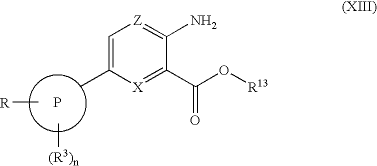

(viii) conversion of a compound of formula III to a compound of formula XIII, wherein X and Z are N or CH, R¹³ is C_{—-6}alkyl and R³ P and n are as defined above, may be carried out by a de-halogen coupling with a suitable compound of formula XXIX.

The reaction may be carried out by coupling of a compound of formula III with an appropriate aryl boronic acid or a bomic ester of formula XXIX. The reaction may be carried out using a suitable palladium catalyst such as Pd(PPh₃)₄, Pd(dppf)Cl₂ or Pd(OAc)₂ together with a suitable ligand such as P(tert-butyl)₃ or 2-(dicyclohexylphosphino)biphenyl or a nickel catalyst such as nickel on charcoal or Ni(dppe)Cl₂ together with Zn and sodium triphenylphosphinetrimetasulfonate. A suitable base such as an alkyl amine e.g. triethyl amine, or potassium carbonate, sodium carbonate, sodium hydroxide or cesium fluoride may be used in the reaction, which is performed in a temperature range between +20° C. and +160° C. using an oil bath or a microwave oven in a suitable solvent or solvent mixture such as toluene, tetrahydrofuran, dimethoxyethane/water or N,N-dimethylformamide.

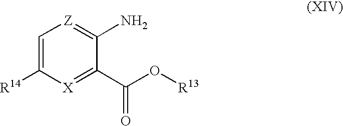

(ix) reaction of a compound of formula XIV, wherein X, Z and R¹³ is as defined above and R¹⁴ is as defind belove, to obtain a compound of formula XIII may be carried out by reacting a compound of formula XIV with a suitable aryl halide. The reaction may be carried out using a suitable palladium catalyst such as Pd(PPh₃)₄, Pd(dppf)Cl₂ or Pd(OAc)₂ together with a suitable ligand or a nickel catalyst such as nickel on charcoal or Ni(dppe)Cl₂ together with Zn and sodium triphenylphosphinetrimetasulfonate. A suitable base such as an alkyl amine e.g. triethyl amine, or potassium carbonate, sodium hydroxide or cesium fluoride may be used in the reaction, which is performed in a temperature range between +20° C. and +120° C. in a suitable solvent such as toluene, tetrahydrofuran or N,N-dimethylformamide.

(x) conversion of a compound of formula XTII, wherein R¹³ is C₁₋₆alkyl, to a compound of formula XIII, wherein R¹³ is hydrogen, may be carried out in a suitable solvent such as tetrahydrofuran or water or mixtures thereof in the presence of a suitable base such as potassium carbonate, sodium hydroxide or lithium hydroxide at a reaction temperature between +20° C. and +60° C.

(xi) borylation of a compound of formula III to a compound of formula XIV, wherein X and Z are N or CH and R¹⁴ may be a group outlined in Scheme II, R¹⁵ and R¹are C₁₋₆alkyl or C₁₋₃alkyl fused together to form a 5 or 6 membered boron-oxygen-C₂₋₃cycloalkyl and the alkyl, cycloalkyl, and the aryl moieties may be optionally substituted, may be carried out by a reaction with:

a) butyllithium or magnesium and a suitable boron compound-such as trimethyl borate or triisopropyl borate. The reaction may be performed in a suitable solvent such as tetrahydrofuran, hexane or methylene chloride in a temperature range between −78° C. and +20° C.; or,

b) a palladium catalyst such as palladium tetrakistriphenylphosphine, palladium diphenylphosphineferrocene dichloride or palladium acetate with or without a suitable ligand such as 2-(dicyclohexylphosphino)biphenyl, and a suitable boron species such as biscatecholatodiboron, bispinacolatodiboron or pinacolborane. A suitable base, which under the reaction conditions do not promote dimerisation of a compound of formula III, such as a tertiary amine such as trietylamine or diisopropylethylamine or potassium acetate may be used. The reaction may be performed in a solvent such as dioxane, toluene or acetonitrile at temperatures between 480° C. and +100° C.

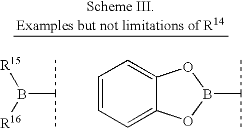

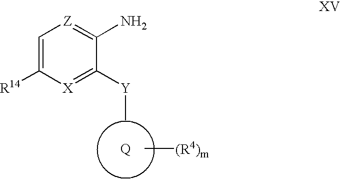

(xii) borylation of a compound of formula XI to a compound of formula XV, wherein X and Z are N or CH, Y is CONR⁵, Q, R⁴ and m are as defined above and R¹⁴ is a group outlined in Scheme II, may be carried out by the reaction conditions described in (xi):

(xiii) amidation of a compound of formula XIV, wherein X and Z are N or CH, R¹³ is C₁₋₆ alkyl and R¹⁴,Q, R⁴ and m are as defined above, to obtain a compound of formula XV, wherein Y is CONR⁵ and may be carried out by reacting a compound of formula XIV with a suitable amine such as a compound of formula X or 3-aminopyridine, under reaction conditions described in (v) and (vi).

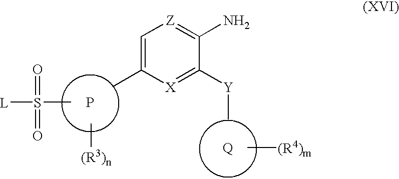

(xiv) conversion of a compound of formula XI to a compound of formula XVI, wherein L is a leaving group such as outline d in Scheme III and Y is CONR⁵ and R³, R⁴, m and n are as defined above, may be carried out by a de-halogen coupling with a suitable aryl species using the conditions described in (viii). The suitable arylSO₂-L species may be prepared by known methods described in the literature.

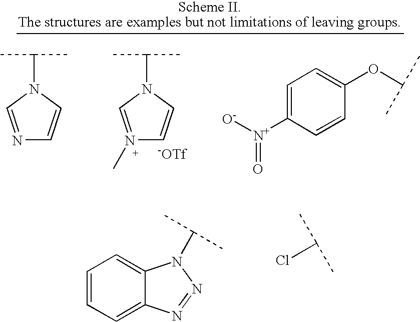

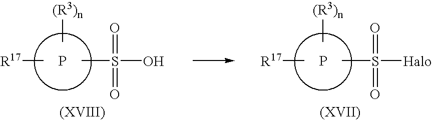

(xv) halogenating a compound of formula XVII, wherein R¹⁷ is bromine, NH₂ or CH₃(CO)NH and P, R³ and n are as defined above, to obtain a compound of formula XVII may be carried out by treatment of a compound of formula XVIII with a halogenation reagents such as thionyl chloride or oxalyl chloride. The reaction may be performed neat or in a suitable solvent such as tetrahydrofaran, dioxane, N,N-dimethylformamide or is methylene chloride at a temperature range between −20° C. and +60° C.;

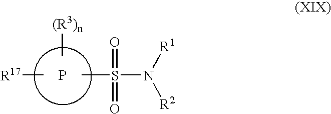

(xvi) amidation of a compound of formula XVII, wherein R¹⁷ is bromine, NH2 or CH₃(CO)NH, halo is fluorine, chlorine or bromine and P, R³ and n are as defined above, to obtain a compound of formula XIX, wherein R¹⁷ is bromine, NH₂ or CH₃(CO)NH and P, R¹, R², R³ and n are as defined above, may be carried out by reacting a compound of formula XVII with the suitable amine HNR¹R². The reaction may be performed in a suitable solvent such as tetrahydrofuran, dioxane, N,N-dimethylformamide or methylene chloride in a temperature range between 0° C. and +50° C.

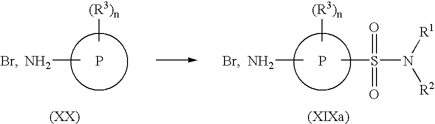

(xvii) conversion of a compound of formula XX, wherein P, R³ and n are as defined above to obtain a compound of formula XIXa, wherein P, R¹, R², R³ and n are as defined above may be carried out by treating a compound of formula XX with a sulfonating reagent such as.chloro sulfonic acid followed by addition of a suitable amine, HNR¹R². The reaction may be performed neat or in an appropriate solvent such as tetrahydrofuran, methylene chloride and at a reaction temperature between 25° C. and reflux.

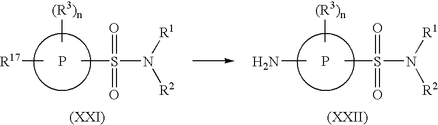

(xviii) transformation of a compound of formula XXI, wherein R¹⁷ is CH₃(CO)NH, and R¹, R², R³, n and P are as defined above, to a compound of formula XXII may be carried out by the reaction with an acid such as hydrochloric acid or hydrobromic acid at a temperature range between +25° C. and +110° C.

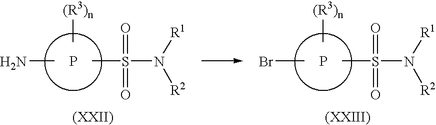

(xix) conversion of a compound of formula XXII to obtain a compound of formula XXIII, wherein R¹, R², R³, n and P are as defined above, may be carried out by treatment of a compound of formula XXII with sodium nitrite and hydrobromic acid followed by the addition of bromide source such as CuBr in an appropriate solvent such as water at a temperature range between 0° C. and +5° C.

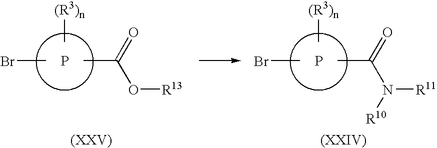

(xx) formation of an amide of formula XXIV, wherein R¹, R² R³, n and P are as defined above, may be carried out by treating a compound of formula XXV, wherein R¹³ is C₁₋₆ alkyl, with the appropriate amine HNR¹OR¹¹. The reaction can be performed neat or using a suitable solvent such as N,N-dimethylformamide, methylene chloride or ethyl acetate at a temperature ranging from −25° C. to +150° C. The reaction may be aided by: using a base such as potassium carbonate, trietylamine or 1,8-diazabicyclo[5.4.0]undec-7-ene or an acid such as trimethylaluminum or p-toulenesulfonic acid.

(xxi) amidation of a compound of formula XXV, wherein R¹³ is hydrogen and R³, n and P are as defined above to obtain a compound of formula XXV may be performed by activation of a compound of formula V by treating the compound with coupling reagents such as 1-[3-(dimethylamino)propyl]-3-ethylcarbodiimide hydrochloride and 1-hydroxybenzotriazole hydrate, 1,3-dicyclohexylcarbodiimide and 1-hydroxybenzotriazole hydrate, 1,1′-carbonyldiimidazole or O-(7-azabenzotriazol-1-yl)-N,N,N′,N′-tetramethyluronium hexafluorophosphate or using an acyl halide reagent such as cyanuric chloride, oxalyl chloride, thionyl chloride or brbmotrispyrrolidinophosphonium hexafluorophosphate, followed by treatment with the appropriate amine HNR¹⁰R¹¹. The reaction may be carried out in a suitable solvent such as N₂N-dimethylformamide, acetonotrile or methylene chloride at a temperature ranging from −25° C. to +150 DC, with or without a suitable base such as an alkyl amine e.g. triethylamine, ethyl diisopropyl amine or N-methyl morpholine, or potassium carbonate or sodium hydroxide.

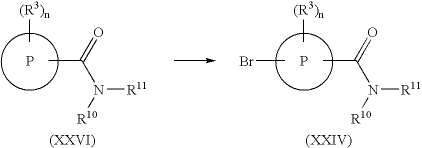

(xxii) bromination of a compound of formula XXVI to obtain a compound of formula XXIV, wherein R¹, R², R³, n and P are as defined above, may be carried out by treatment of a compound of formula XXVI with bromine with or without an appropriate base such as sodium acetate in a suitable solvent such as acetic acid.

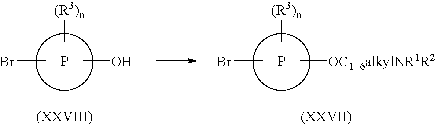

(xxiii) conversion of a compound of formula XXVIII, wherein R³, n and P are as defined above, to obtain a compound of formula XXVII, wherein R¹, R², R³, n, C₁₋₆alkyl and P are as defined above, may be carried out by reacting a compound of formula XXVIII with a suitable alcohol, R¹R²C₁₋₆alkylOH in the presence of triphenylphosphine and an appropriate azidodicarboxylate such as diethyl azidodicarboxylate. The reaction may be performed in a suitable solvent such as tetrahydrofuran, toluene or methylene chloride and at a reaction temperature between 0° C. to 60° C.

(xxiv) conversion of a compounds of formula XXIII, XXIV and XXVII to obtain compounds of formula XXIX, wherein R¹⁴, R¹, R², R³, R¹⁰, R¹¹, n, C₁₋₆alkyl and P are as defined above, may be carried out by a borylation reaction described in (xi)

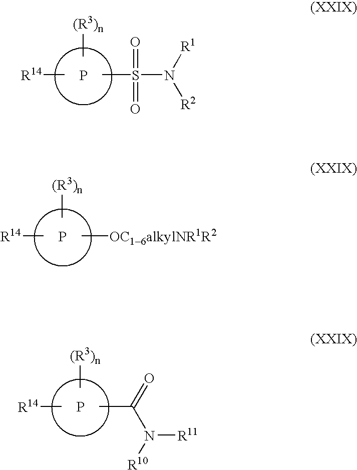

Methods of preparation of the End Products.

Another object of the invention are processes for the preparation of a compound of general formula I, wherein Y, X, Z, P, Q, R¹, R², R³,, R³, R⁵, R⁶, R⁷, R⁸, R⁹. R¹⁰, R¹¹, R¹², A, m and n are, unless specified otherwise, defined as in formula I, comprising of

A

de-halogen coupling, wherein R³ and R⁴ are substituents that are not susceptible to certain agents in the reaction, of a compound of formula XI with a suitable aryl species to give a compound of formula I:

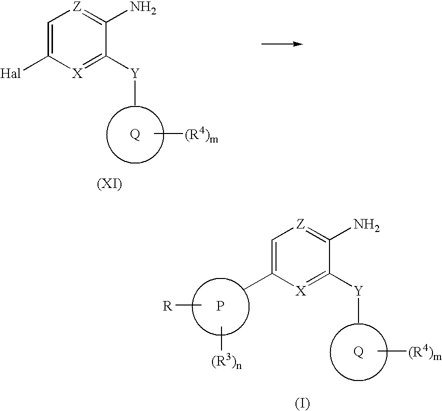

Thus, the de-halogen coupling according to process A may be carried out by coupling of a compound of formula XI with:
a) an appropriate aryl halogen such as aryl iodide, aryl bromide or aryl chloride in the presence of metals such as copper, nickel or zink and nickel complexes, copper oxide or palladium acetate and tetrabutyiammonium bromide and a base such as potassium carbonate or trietylamine. The reaction may occur at a temperature between 20 DC and 180° C. in a suitable solvent such as N,N-dimetylformamide, toluene or 2-pentanol;
   or,
b) an appropriate aryl boronic acid or a bornic ester such as compounds of formula XXIX. The reaction may be carried out using a suitable palladium catalyst such as Pd(PPh₃)₄, Pd(dppf)Cl₂ or Pd(OAc) together with a suitable ligand such as P(tert-butyl)₃ or 2-(dicyclohexylphosphino)biphenyl or a nickel catalyst such as nickel on charcoal or Ni(dppe)Cl₂ together with Zn and sodium triphenylphosphinetrimetasulfonate. A suitable base such as an alkyl amine e.g. triethyl amine, or potassium carbonate, sodium carbonate, sodium hydroxide or cesium fluoride may be used in the reaction, which is performed in the temperature range between +20° C. and +160° C. using an oil bath or in a microwave oven in a suitable solvent or solvent miixtuire such as toluene, tetrahydrofuran, dimethoxyethane/water or N,N-dimethylformamide;
   or,
c) an appropriate aryl stannane in the presence of palladium catalyst such as Pd(PPh₃)₄, Pd(PPh₃)₂Cl₂ or Pd(dba)₃ and if needed a helping reagent such as 4-tert-butylcatechole, lithium chloride or potassium carbonate. Suitable solvents may be toluene, tetrahydrofuran or N,N-dimethylformamide. The reaction may occur in a temperature range of +20° C. and +120° C.;
   or,
d) an appropriate aryl halogen such as aryl iodide or aryl bromide by treatment with butyllithium in a suitable solvent such as tetrahydrofuran at a reaction temperature between 78° C. and −25° C., and a suitable base such as sodium carbonate or potassium carbonate in the presence of a suitable palladium catalyst such as Pd(dppf)Cl₂ or Pd(OAc)₂ and at a reaction temperature between 25° C. and reflux.
   B
   amidation, wherein R³ and R⁴ are substituents that are not susceptible to certain agents in the reaction, of a compound of formula XIII with the appropriate amine:

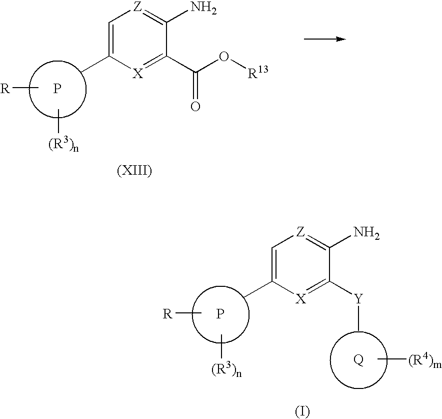

Thus, the amidation according to process B may be carried out by treating a compound of formula XIII, wherein R¹³ is C₁-C₆alkyl, with the appropriate amine such as a compound of formula X or 3-aminopyridine. The reaction can be performed neat or using a suitable solvent such as N,N-dimethylformamide, methylene chloride or ethyl acetate at a temperature ranging from −25° C. to +150° C. The reaction may be aided by using a base such as potassium carbonate, triethylamine or 1,8-diazabicyclo[5.4.0]undec-7-ene or an acid such as trimethylaluminum or p-toulenesulfonic acid;

or,

the amidation of a compound of formula XIII, wherein R¹³ is hydrogen, may be performed by activation of a compound of formula XIII by treating the compound with coupling reagents such as 1-[3-(dimethylamino)propyl]-3-ethylcarbodimide hydrochloride and 1-hydroxybenzotriazole hydrate, 1,3-dicyclohexylcarbodiimide and 1-hydroxybenzotriazole hydrate, 1,1′-carbonyldiimidazole or O-(7-azabenzotriazol-1-yl)-N,N,N′,N′-tetramethyluronium hexafluorophosphate or using an acyl halide reagent such as cyanuric chloride, oxalyl chloride, thionyl chloride or bromotrispyrrolidinophosphonium hexafluorophosphate followed by treatment with the appropriate amine such as a compound of formula X or 3-aminopyridine.

C

de-halogen coupling, wherein R³ and R⁴ are substituents that are not susceptible to certain agents in the reaction, of a compound of formula XV with an appropriate aryl species to give a compound of formula I,

wherein R¹⁴ IS 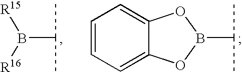
and R¹⁵ and R¹⁶ are C₁₋₆alkyl or C₁₋₃alkyl fused together to form a 5 or 6 membered boron oxygen-C₂-C₃cycloalkyl and the alkyl, cycloalkyl and the aryl moieties may be optionally substituted;

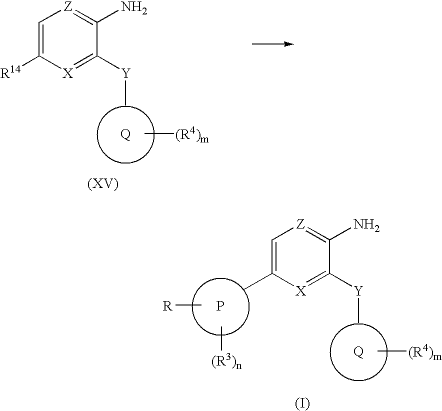

Thus, the de-halogen coupling according to process C may be carried out by using a suitable palladium catalyst such as Pd(PPh₃)₄, Pd(dppf)Cl₂ or Pd(OAc)₂ together with a suitable ligand such as P(tert-butyl)₃ or 2-(dicyclohexylphosphino)biphenyl, or a nickel catalyst such as nickel on charcoal or Ni(dppe)Cl₂ together with Zn and sodium triphenylphosphinetrimetasulfonate in the precense of a suitable aryl bromide, aryl iodide or aryl chloride. A suitable base such as an alkyl amine e.g. triethyl amine, or potassium carbonate, sodium hydroxide or cesium fluoride may be used in the reaction, which is performed in the temperature range between +20° C. and +120° C. in a suitable solvent such as toluene, tetrahydrofuran or N,N-dimethylform amide.

D

reaction of a compound of formula XVI, wherein L is a leaving group with an appropriate amine, to give a compound of formula Ia:

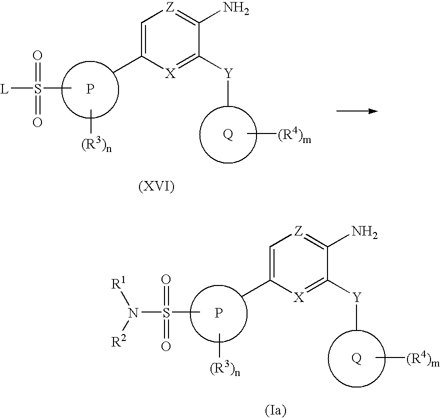

Thus, the reaction according to process D may be carried out by treating a compound of formula XVI with the appropriate amine HNR¹R², in a suitable solvent such as tetrahydrofuran, methanol or water at temperatures in the range of 0° C. and +80° C. with or without a suitable base such as an alkylamine such as triethyl amine, sodium hydroxide or potassium carbonate.

E

amidation, wherein R³ and R⁴ are substituents that are not susceptible to certain agents in the reaction, of a compound of formula Ib, with the appropriate amine to give a conpound of formula Ic:

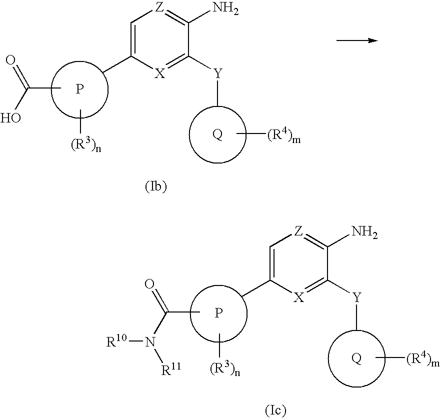

Thus the amidation of a compound of formula I according to process E may be performed by activation of the carboxylic acid function in a compound of formula Ib, wherein R is COOH, by treating the compound with coupling reagents such as 1-[3-(dimethylamino)propyl]-3-ethylcarbodiimide hydrochloride and 1-hydroxybenzotriazole hydrate, 1′,3-dicyclohexylcarbodiimide and 1-hydroxybenzotriazole hydrate, 1,1′-carbonyldiimidazole or O-benzotriazol-1-yl-N,NN′,N′-tetramethyluronium hexafluorophosphate or using an acyl halide reagent such as cyanuric chloride, oxalyl chloride, thionyl chloride or bromotrispyrrolidinophosphonium hexafluorophosphate in a suitable solvent such as N,N-dimethylformamide, dioxane or tetrahydrofuran followed by treatment with the appropriate amine HNR¹⁰R¹¹ and at a reaction temperature between 25° C. and 70° C.

The hydrochloric salt of compound of formula I may be obtained from a compound of formula I by treatment with hydrochloric acid at a temperature range between 0° C. and +25° C., in suitable solvent such as methylene chloride, tetrahydrofuran or methylene chloride/methanol mixture.

### EXAMPLES

The following examples will now be illustrated by the following non-limiting examples.

General Methods

All starting materials are commercially available or earlier described in the literature. The ¹H and ¹³C NMR spectra were recorded on Brucker 400 at 400 Mz and 0.100 MHz, respectively. The mass spectra were recorded utilising thermospray (Finnigan MAT SSQ 7000, buffer: 50 nM NH₄OAc in CH₃CN:H₂O; 3:7), electron impact (Finnigan MAT SSQ 710) or electrospray (LC-MS; LC:Waters 2790, column XTerra MS C₈ 2.51 μm 2.1×30 mm, buffer gradient H₂O+0.1% TFA:CH₃CN+0.04% TFA, MS: micromass ZMD) ionisation techniques.

### Example 1

### 3-Amino-6-bromo-N-pyridin-3-ylpyrazine-2-carboxamide

To 3-aminopyridine (10 g, 106 mmol) at 70° C. were added methyl 3-amino-6bromo-2-pyrazinecarboxylate (1.0 g, 4.3 mmol) and 1,8-diazabicyclo[5.4.0]undec-7-ene (645 μL, 4.3 mmol). The reaction solution was stirred for 4 h, diluted with water (75 mL) and extracted with methylene chloride. The combined organic layers were washed with a saturated ammonium chloride solution, dried (MgSO₄), filtered and evaporated in vacuo. The crude product was purified on a silica gel column using methylene chloride/ethanol, (9:1), as the eluent to give-750 mg (59% yield) of the title compound as a yellow solid: ¹H NMR (CDCl₃, 400 MHz) δ 9.50 (br s, 1H), 8.82 (d, J=3 Hz, 1H), 8.43 (dd, J=5 and 1 Hz, 1H), 8.31 (s, 1H), 8.23 (ddd, J=8, 3 and 2 Hz, 1H), 7.34 (dd, J=8, 5 Hz, 1H); MS (TSP) m/z 294 (M⁺+1).

### Example 2

### N,N-Dimethyl-4-(4,4,5,5-tetramethyl-[1,3,2]-dioxaborolan-2-yl)benzenesulfonamide

A three necked round bottom flask equipped with a dripping funnel and a condenser was to charged with bispinacolatodiborone (508 mg, 20 mmol), Pd(dppf)Cl₂:CH₂Cl₂; 1:1 (4.9 mg, 6 lmol) and potassium acetate (59.9 mg, 0.6 mmol). The system was evacuated and nitrogen atmosphere was introduced. N,N-dimethylformamide (SnL) was added and the mixture was stirred at 80° C. A solution of 4-bromo-(N,N-dimethyl)benzenesulfamide (52.8 mg, 0.2 mmol) in N,N-dimethylformamide (5 mL) was added to the reaction mixture is via the dripping funnel during 30 min. After 4 h, the solvent was removed in vacuo and the residue was partitioned between ethyl acetate and 1 M HCl (aq) and the layers were separated. To the organic layer was added 6.5 g silica gel and the mixture was concentrated to dryness. The residue was purified on a silica gel column using heptane/ethyl acetate, (5:1), as the eluent to give 31 mg (50% yield) of the title compound as a white solid: ¹H NMR (CDCl₃, 400 MHz) 7.95 (d, J=8 Hz, 2H), 7.74 (d,f=8 Hz, 2H), 2.67 (s, 6H), 1.34 (s, 12H).

### Example 3

### N,N-Dimethyl-3-(4,4,5,5-tetramethyl-1,3,2-dioxaborolan-2-yl)benzenesulfonamide

The compound was prepared as described for Example 2 using 3-bromo-N,N-dimethylbenzenesulfonamide: yield 66. %; 3H NMR (CDCl₃, 400 MHz) 8.20 (br s, 1H), 8.01 (br d, J=8 Hz, 1H), 7.87-7.84 (m, 1H), 7.54 (t, J=8 Hz, 1H), 2.72 (s, 6H), 1.13 (s, 12H).

### Example 4

### N,N-Dimethyl-2-(4,4,5,5-tetramethyl-1,3,2-dioxaborolan-2-yl)benzenesulfonamide

The compound was prepared as described for Example 2 using 2-bromo-N,N-dimethylbenzenesulfonamide: yield 14%; MS (ES) m/z 312 (M⁺+1).

### Example 5

### 2-Amino-5-bromo-N-pyridin-3-ylnicotinamide.

2-Amino-5-bromonicotinic acid (0.25 g, 1.15 nmol), 3-aminopyridine (0.22 g, 2.3 mmol), diisopropylcarbodiimide (0.27 mL: 0.22 g, 1.74 mmol), 1-hydroxybenzotriazole hydrate (0.31 g, 2.3 mmol) and N-methylmorpholine (0.38 mL: 0.35 g, 3.8 mmol) were mixed in N,N-dimethylformamide (5 ml) and stirred at room temperature for 4 h. The solvent was evaporated in vacuo and the remaining solid was purified on a silica gel column, using a gradient toluene 100% to ethyl acetate 100% as the eluent, to give 337 mg (52% yield) of the title compound as a solid: ¹H NMR (DMSO-d₆, 400 MHz) δ 6.93 (dd, J=6 Hz, 1H), 7.19 (d, J=2 Hz, 1H), 7.44(d, J=2 Hz, 1H), 7.55 (d, J=7 Hz, 1H), 7.64 (d, J=9 Hz, 1H), 8.20 (s, 1H), 10.30 (s, 1H); ¹³C NMR (DMS O-d6, 400 MHz) δ 103.84, 110.60, 127.21, 133.38, 135.45, 136.96, 138.31, 140.61, 151.18, 156.85, 165.63; MS (MS) m/z 293 and 295 (M⁺+1).

### Example 6

### 4-(4,4,5,5-Tetramethyl-1,3,2-dioxaborolan-2-yl)benzenesulfonamide

The compound was prepared as described for Example 2 using 4-bromobenzenesulfonamide. After 4 h, 0.75 g silica gel was added to the reaction mixture and the solvent was removed in vacuo. The residue was purified on a silica gel column using heptane/ethyl acetate, (5:1->3:1), as the eluent to give the title compound (64% yield) as a yellow solid: mp 240-242 DC; ¹H NMR (DMSO-d₆, 400 MHz) 7.83 (s, 4H), 7.43 (s, 2H), 1.31 (s, 12H); ¹³C NMR (DMSO-d₆, 100 MHz) 134.89, 124.95, 84.20, 24.70; EINS (70 eV) m/z 283 (M+).

### Example 7

### 3-Amino-6-[4-({[2-(dimethylamino)ethyl]amino}sulfonyl)phenyl]-N-pyridin-3-ylpyrazine-2-carboxamide

Triisopropyl borate (2.35 mL, 10.2 mmol) was added to a cooled (−78° C.) solution of 4-bromo-N-[2-(dimethylamino)ethyl]benzenesulfonamide (0.626 g, 2.0 mmol) in anhydrous tetrahydrofuran (30 mL) under nitrogen atmosphere. The solution was treated with n-butyllithium (6.4 mL, 10.2 mmol) dropwise over 35 min. The resulting mixture was stirred at −78° C. for 3.5 h and at room temperature for another 16 h. Water (10 mL) was added, the mixture stirred for 30 min, and evaporated to dryness. The residue was pre-adsorbed onto silica and purified by column chromatography on silica using methylene chloride/methanol, (4:6), to methanol as the eluent to give 540 mg (88% yield) of the title compound as a white foam: ¹H NMR (CD₃OD, 400 MHz) δ 7.70 (d, J=8 Hz, 2H), 7.63 1o (d, J=8 Hz, 2H), 2.94 (t, J=7 Hz, 2H), 2.36 (t, J=7 Hz, 2H), 2.16 (s, 6H); ¹³CNMR (CD₃OD, 100 MHz) δ 137.7, 135.1, 126.2, 59.6, 45.7, 42.0; MS (TSP) m/z 273 (M⁺+1).

### Example 8

### 4-{[(3-Morpholin-4-ylpropyl)amino]sulfonyl}phenylboronic acid

The title compound was prepared as described for-Example 7 using 4-bromo-N-(3-morpholin-4ylpropyl)benzenesulfonamide: yield 54%; ¹H NMR (CD₃OD, 400 MHz) 5-7.70 (d, J=8 Hz, 2H), 7.62 (d, J=8 Hz, 2H), 3.65 (t, J=5 Hz, 4H), 2.89 (t, J=7 Hz, 2 H), 2.38 (m, 4H), 2.34 (m, 2H), 1.62 (m, 2H); ³C NMR (CD₃OD, 100 Mz) δ 137.7, 135.0, 125.9, 67.8, 57.6, 54.8, 42.8, 27.1.

### Example 9

### 4-[(4-Methylpiperazin-1-yl)sulfonyl]phenylboronic acid

Triisopropylborate (0.64 mL, 2.8 mmol) was added to a solution of 1-[(4-bromophenyl)sulfonyl]4-methylpiperazine (0.602 g, 1.9 mmol) in anhydrous 2s tetrahydrofuran (7 mL) at −78° C. under nitrogen atmosphere followed by dropwise addition of n-butyllithium (1.4 nL, 2.2 mmol). The resulting mixture-was stirred at −78° C. for 2 h and at room temperature for another 16 h. Water (2.0 mL) was added, the mixture stirred for 30 min and evaporated to dryness. The residue was pre-adsorbed onto silica and purified by column chromatography using methylene chloride/methanol, (9:1 to 1:9), as the eluent. The product was recrystallized from water to give 311 mg (58% yield) of the title compound as white crystals: mp 215-218° C.; ¹H NMR (DMSO-d₆, 400 MHz) δ 10.89(br s, 1H), 8.47 (br s, 2H), 8.05 (d, J=8 Hz, 2H), 7.73 (d, J=8 Hz, 2H), 3.77 (m, 2H), 3.40 (m, 2H), 3.13 (m, 2H), 2.71 (s, 3H), 2.65 (m, 2H); ¹³C NMR (DMSO-d₆, 100 MHz) 133.7, 133.3, 124.7, 49.8, 41.6, 41.4; MS (TSP) m/z 285 (M⁺+1).

### Example 10

### 4-Bromo-N-[2-(dimethylamino)ethyl]benzenesulfonamide

N,N-Dimethylethylenediamine (0.55 mL, 5.0 mmol) was added to a stirred solution of 4-bromobenzenesulphonyl chloride (0.644 g, 2.5 mmol) in tetrahydrofuran (7.5 μL) and the resulting mixture was stirred at room temperature for 20 min. The solvent was evaporated and the resulting mixture dissolved in ethyl acetate. The organic phase was washed with water and brine, dried over MgSO₄, and the solvent was evaporated to give the title compound as a white solid: yield: 99%; ¹H NMR (CDCl₃, 400 MHz) δ 7.77 (d, J=8 Hz, 2H), 7.68 (d, J=8 Hz, 2H), 3.00 (t, J=6 Hz, 2H), 2.37 (t, J=6 Hz, 2H), 2.12 (s, 6H); ¹³C NMR (CDC₃, 100 MHz) 140.6, 134.1, 130.5, 129.3, 58.7, 46.5,41.8.

### Example 11

### 4-Bromo-N-(3-morpholin-4-ylpropyl)benzenesulfonamide

The title compound was prepared as described for Example 10 using 3-morpholin-4-ylpropan-1-amine: yield: 87%; ¹H NMR (CDCl₃, 400 MHz) δ 7.73 (m, 2H), 7.67 (m; 2H), 7.19 (br s, 1H), 3.73 (t, J=4 Hz, 4H), 3.10 (m, 2H), 2.45 (m, 6H), 1.68 (quint, J=6 Hz, 2H); ¹³C NMR (CDCl₃, 100 MHz) δ 139.7, 132.7, 128.9, 127, 67.3, 58.7, 53.9, 44.4, 24.2.

The following Examples 12-21 were synthesized as described for Example 10:

### Example 12

### 1-[(4-Bromo-2,5-difluorophenyl)sulfonyl]-4-methylpiperazine

Starting material: 4-bromo-2,5-diflourobenzenesulfonyl chloride and 1-methylpiperazine, yield 97%: ¹H NMR (CDCl₃, 400 MHz) δ 7.60 (m, 1H), 7.48 (m, 1H), 3.27 (br s, 4H), 2.53 (br s, 4H), 2.33 (s, 3H); ³C N (CDCl₃, 100 MHz) δ 156.4, 155.8, 155.7, 154.0, 153.9, 153.2, 126.1, 126.0, 125.9, 125.9, 122.8, 122.5, 118.4, 118.1, 115.5, 54.3, 45.9, 45.7.

### Example 13

### 1-[(4-Bromo-2-ethylphenyl)sulfonyl]-4-methylpiperazine

Starting material: 4-bromo-2-ethylbenzenesulfonyl chloride and 1-methylpiperazine, yield 97%: ¹H NMR (CDCl₃, 400 MHz) δ 7.74 (d, J=9 Hz, 1H), 7.53 (d, J=2 Hz, 1H), 7.45 (dd,J 8,2 Hz, 1H), 3.20 (t, J=5 Hz, 4H), 2.98 (q, J=8 Hz, 2H), 2.47 (t, J=5 Hz, 4H), 2.30 (s, 3H), 1.28 (t, J=8 Hz, 3H); ¹³C NMR (CDCl₃, 100 MHz) δ 146.4, 134.3, 143.2, 132.0, 129.3, 128.1, 54.4, 46.0, 45.3, 26.2, 15.7.

### Example 14

### 1-{[4-Bromo-2-(trifluoromethoxy)phenyl]sulfonyl}-4-methylpiperazine

Starting material: 0.4-bromo-2-(trifluoromethoxy)benzenesulfonyl chloride and 1-methylpiperazine, yield 96%: H N (CDCl₃, 400 MHz) δ 7.84 (d, J=8 Hz, 1H), 7.55 (m, 2H), 3.27 (m, 4H), 2.50 (m, 4H), 2.32 (s, 3H); ³C NMR (CDCl₃ 100 MHz) δ 146.5, 146.5, 133.0, 130.1, 129.6, 128.4, 124.2, 124.2, 121.5, 118.9, 116.3, 54.5, 45.9, 45.7.

### Example 15

### 1-[(4-Bromo-2-fluorophenyl)sulfonyl)-4-methylpiperazine

Starting material: 2-bromo4-fluorobenzenesulfonyl chloride and 1-methylpiperazine, yield 99%: ¹H NMR (CDCl₃, 400 MHz) δ 7.69 (m, 1H), 7.42 (m, 2H), 3.22 (m, 4H), 2.50 (m, 4H), 2.30 (s, 3H); N³CNMR (CDCl₃, 100 MHz) δ 160.1, 157.5, 132.3, 128.7, 128.6, 128.2, 124.4, 124.3, 121.5, 121.0, 54.3,45.9, 45.7; MS (TSP) m/z 337 and 339 (M⁺+1).

### Example 16

### 1-[(4-Bromo-2-methylphenyl)sulfonyl]-4-methylpiperazine

Starting material: 2-bromo-4-methylbenzenesulfonyl chloride and 1-methylpiperazine, yield 99%: ¹H NMR (CD Cl₃, 400 MHz) 87.74 (d, J=8 Hz, 1H), 7.46 (m, 2H), 3.20 (m, 4H), 2.59 (s, 3H), 2.47 (m, 4H), 2.30 (s, 3H); ¹³C N (CDCl₃, 100 MHz) δ 140.2, 135.8, 134.8, 131.9, 129.5, 127.8, 54.4, 46.0, 45.2, 20.8; MS (TSP) m/z 333 and 335′ (M⁺+1).

### Example 17

### 1-[(2-Bromophenyl)sulfonyl]-4-methylpiperazine

Starting material: 2-bromobenzenesulfonyl chloride and 1-methylpiperazine, yield 97%: H NMR (CDCl₃, 400 MHz) δ 8.07 (dd, J=8, 2 Hz, 1H), 7.75 (d, J=8 Hz, 1H), 7.43 (m, 2H), 3.39 (br s, 4H), 2.55 (br s, 4H), 2.35 (s, 3H); ¹³C NMR (CDCl₃, 100 MHz) δ 137.6, 136.1, 133.9, 132.4, 127.7, 120.7, 54.5, 45.9, 45.5; MS (TSP) m/z 319 and 321 (M⁺+1).

### Example 18

### 1-[(3-Bromophenyl)sulfonyl]-4-methylpiperazine

Starting material: 3-bromobenzenesulfonyl-chloride and 1-methylpiperazine, yield 86%:

H NMR (CDCl₃, 400 MHz) δ 7.89 (m, 1H), 7.71 (m, 2H), 7.42 (r, 1H), 3.11 (br s, 4H), is 2.57 (br s, 4H), 2.33 (s, 3H); ¹³CNMR (CDCl₃, 100 MHz) δ 137.4, 136.2, 130.8, 130.7, 126.5, 123.4,54.0,45.9, 45.7; MS (TSP) m/z 319 and 321 (M⁺+1)

### Example 19

### 4-Bromo-N-[2-(dimethylamino)ethyl]-2-(trifluoromethoxy)benzeneulfonamde

Starting material: 4-bromo-2-(trifluoromethoxy)benzenesulfonyl chloride and N,N-dimethylethylenediamine, yield 99%: ¹H NMR (CDCl₃, 400 MHz) δ 7.91 (d, J=9 Hz, 1H), 7.56 (m, 2H), 3.03 (m, 2H), 2.40 (m, 2H), 2.17 (s, 6H); ¹³C NMR (CDCl₃, 100 MHz) “146.4, 132.3, 131.0, 129.8, 128.1, 123.1, 123.1, 121.6, 119.0, 57.3, 44.9, 40.4.

### Example 20

### 4-Bromo-N-[2-(dimethylamino)ethyl]-N-ethyl-2-(trifluoromethoxy)benzenesulfonamide

Starting material: 4-bromo-2-(trifluoromethoxy)benzenesulfonyl chloride and N,N-dimethyl-N′-ethylethylenediamine, yield 98%: ¹H NMR (CDCl₃, 400 MHz) δ 7.90 (d, J 9 Hz, 1H), 7.51 (m, 2H), 3.40 (t, J=7Hz,2H), 3.33 (q, J=7 Hz, 2H), 2.52 (t, J=7 Hz, 2H), 2.24 (s, 0.6H), 1.09 (t, J=7 Hz, 3H); ¹³cNMR(CDC₃, 100 MHz) δ 146.3, 132.8, 132.1, 129.8, 127.9, 123.5, 123.5, 121.6, 119.0, 58.1, 45.5, 44.9, 43.2, 14.2.

### Example 21

### N-(2-Aminoethyl)-4-bromo-2-(trifluoromethoxy)benzenesulfonamide

Starting material: 4-bromo-2-(trifluoromethoxy)benzenesulfonyl chloride and ethylenediamine, yield 89%: ¹H NMR (CD₃OD, 400 MHz) δ 7.91 (m,n 1H), 7.71 (m, 2H), −2.98. (t, J=6 Hz, 2H), 2.67 (t, J=6 Hz, 2H); ¹³C NMR (CD₃OD, 100 MHz) δ 147.6, 134.0, 133.3, 131.7, 128.8, 125.5, 123.1, 120.5, 46.6, 42.5.

### Example 22

### tert-Butyl 2-({[4-bromo-2-(trifluoromethoxy)phenyl]sulfonyl}-(tert-butoxycarbonyl)amino)ethylcarbamate

4-Dimethylaminopyridine (0.025 g, 0.20 mmol) and di-tert-butyl dicarbonate (0.815 g, 3.73 mmol) was added to a stirred solution of N-(2-aminoethyl)-4-bromo-2-(trifluoromethoxy)benzenesulfonamide (0.644 g, 1.77 mmol) in tetrahydrofuran (20 mL) and the resulting mixture was stirred at reflux-for 45 min. The solvent was evaporated and the crude product purified by column chromatography on silica using heptane/ethyl acetate, (3:1), to give 0.94 g (94% yield) of the title compound: ¹H NMR (CDCl₃, 400 MHz) δ 7.99 (m, 1H), 7.55 (m, 2H), 4.89 (br s, 1H), 3.94 (m, 2H), 3.44 (m, 2H), 1.43 (s, 9H), 1.31 (s, 9H).

### Example 23

### 4-Bromo-N-methyl-N-(1-methylpyrrolidin-3-yl)benzenesulfonamide

A solution of methyl-(1-methylpyrrolidin-3-yl)amine (0.89 g, 7.8 mmol) in dioxane (5 mL) was added dropwise to a solution of 4bromobenzenesulfonyl chloride (2.0 g, 7.8 mmol) in dioxane (5 mL) under vigorous stirring and cooling on ice-bath. The mixture was stirred 30 min, and then diluted with ethyl acetate (10 mL). The precipitated material was filtered off, washed with ethyl acetate (10 mL) and dried in vacuo. The solid was dissolved in water, alkalyzed with sodium hydroxide (2 M, aq) and extracted with ethyl acetate three times. The ethyl acetate phases were dried (Na₂SO₄) and evaporated in vacuo to afford 2.5 g (96% yield) of a clear oil: MS (ES) rr/z 333 and 335 (M⁺+1).

The following Examples, 24-38, were synthesized as described for Example 23:

### Example 24

### 4-Bromo-N-[2-(dimethylamino)-1-methylethyl]benzenesulfonamide

Starting material: 2-(dimethylamino)-1-methylethylamine: MS (ES) m/z 321 and 323 (M⁺+1).

### Example 25

### 4-Bromo-N-(3-pyrrolidin-1-ylpropyl)benzenesulfonamide

Starting material: 3-pyrrolidin-1-ylpropylamine: MS (ES) m/z 347 and 349 (M⁺+1).

### Example 26

### 1-Acetyl-4-[(4-bromophenyl)sulfonyl]piperazine

A solution of 1-N-acetylpiperazine (1 g, 7.8 mmol) and triethylamine (1 mL, 7.8 mmol) in dioxane (5 mL) was added dropwise to a solution of 4-bromobenzenesulfonyl chloride (2.0 g, 7.8 mmol) in dioxane (5 XhL) under vigorous stirring and cooling with ice. The mixture was stirred 48 h. The filtrate was concentrated under reduced pressure to give 1.98 g (73% yield) of the title compound as an oil: MS (ES) m/z 347 and 349 (M⁺+1).

### Example 27

### 4-Bromo-N-methyl-N-(1-methylpiperidin-4-yl)benzenesulfonamide

Starting material: methyl-(1-methylpiperidin-4-yl)amine: MS (ES) m/z 347 and 349 (M⁺+1).

### Example 28

### 4-Bromo-N-[3-(dimethylamino)propyl]-N-methylbenzenesulfonamide

Starting material: N,N,N′-trimethylpropane-1,3-diamine: MS (ES) m/z 335 and,337 (M⁺+1).

### Example 29

### 4-Bromo-N-[2-(dimethylamino)ethyl]-N-ethylbenzenesulfonamide

Starting material: N′-ethyl-N,N-dimethylethane-1,2-diamine: MS (ES) m/z 335 and 337 (M⁺+1).

### Example 30

### 4-Bromo-N-[3-(4-methylpiperazin-1-yl)propyl]benzenesulfonamide

Starting material: 3-(4-methylpiperazin-1-yl)propylamine: MS (ES) m/z 376 and 378

### Example 31

### 1-[(4-Bromophenyl)sulfonyl]-4-ethylpiperazine

Starting material: 1-ethylpiperazine (diethyl ether was used instead of ethyl acetate): MS (ES) m/z 333 and 335 (M++I).

### Example 32

### 4-Bromo-N-(2-pyrrolidin-1-ylethyl)benzenesulfonamide

Starting material: 2-(pyrrolidin-1-yl)ethylamine: MS (ES) m/z 333 and 335 (M⁺+1).

### Example 33

### 1-[(4-Bromophenyl)sulfonyl]-4-methyl-1,4-diazepane

Starting material: 1-methyl-1,4-diazepane: MS (ES) rr/z 333 and 335 (M⁺+1).

### Example 34

### 4-Bromo-N-[2-(-dimethylamino)propyl]benzenesulfonamide

Starting material: 2-dimethylaminopropaneamine: MS (ES) m/z 321 and 323 (M⁺+1).

### Example 35

### 4-Bromo-N-[(1-ethylpyrrolidin-2-yl)methyl]benzenesulfonamide

Starting material: (1-ethylpyrrolidin-2-yl)methylamine: MS (ES) m/z 347 and 349 (M⁺+1).

### Example 36

### 4-Bromo-N-[2-(diethylamino)ethyl]benzenesulfonamide

Starting material: N,N-diethylethane-1,2-diamine: MS (ES) m/z 335 and 337 (M⁺+1).

### Example 37

### 4-Bromo-N-(2-pyridin-2-ylethyl)benzenesulfonamide

Starting material: 2-pyridin-2-ylethylamine. The crude product was purified on a silica gel column using methanol/methylene chloride, (1:10), as the eluent: MS (ES) In/z 341 and 343 (M⁺+1).

### Example 38

### 4-Bromo-N-[3-(dimethylamino)propyl]benzenesulfonamide

Starting material: N,N-methylpropane-1,3-diamine: MS (ES) m/z 321 and 323 (M⁺+1).

### Example 39

### 1-[(4-Bromophenyl)sulfonyl]-N,N-dimethylpyrrolidin-3-amine

A solution of dimethylpyrrolidin-3-ylamine (0.89 g, 7.8 mmol) in dioxane (5 mL) was added dropwise to a solution of 4-bromobenzenesulfonyl chloride (2.0 g, 7.8 mmol) in dioxane (5 mL) under vigorous stirring and cooling on ice-bath. The mixture was stirred 30 min, and then diluted with diethyl ether (10 mL). The mixture was filtered and evaporation of the filtrate gave 2.6 g of a brown oil: MS (ES) m/z 333 and 335 (M⁺+1).

The following Examples, 40-42, were synthesized as described for Example 39:

### Example 40

### 4-[(4-Bromophenyl)sulfonyl]morpholine

Starting material: morpholine. The title compound crystallized from the filtrate as long 0.25 needles: MS (ES) m/z 306 and 308 (M⁺+1).

### Example 41

### 4-Bromo-N-isopropyl-N-(2-methoxyethyl)benzenesulfonamidce

Starting material: isopropyl-(2-methoxyethyl)amine: MS (ES) m/z 336 and 338 (M⁺+1).

### Example 42

### 4-Bromo-N-(2-methoxy-1-methylethyl)benzenesulfonamide

Starting material: 2-methoxy-1-methylethylamine. The crude product was purified on a silica gel column using hexane/ethyl acetate, (4:1): MS (ES) m/z 308 and 310 (M++I).

### Example 43

### 4-Bromo-N-[2-(dimethylamino)ethyl]benzamide

A mixture of p-bromobensoic acid (1 g, 4.97 mmol) in thionyl chloride (10 mL) was refluxed for 10 inin and then cooled to room temperature and the thionyl chloride was evaporated in vacuo. The residue was dissolved in methylene chloride (10 mL) and the solution was cooled to 0° C. 2-Dimethylaminoethylamine (0.52 mL, 4.73 mmol) was added dropwise and the mixture was stirred at room temperature for 24 h. The mixture was acidified with 1 M HCl and washed with methylene chloride. The water phase was alkalized with 1 M NaOH (aq) and extracted with methylene chloride. The combined organic phases were dried (MgSO₄) and the solvent was removed in vacuo affording 1.07 g (84% yield) of the title compound as a white solid: mp 67-69° C.; ¹H NMR (DMSOd6, 400 MHz) 8.06 (d, J=8 Hz, 2H), 7.98 (d, J=8 Hz, 2H), 3.67 (t, 2H), 2.80 (s, 6H), 2.49 (s, 2H); ¹³C NMR (DMSO-d₆, 100 MHz) δ 165.2, 133.6, 131.3, 129.3, 124.9, 58.0, 45.2, 37.3; MS (EI) 7z/z 273 (M⁺+1).

### Example 44

### 4-Bromo-N-[2-(dimethylamino)ethyl]-N-methylbenzamide

The title compound was prepared as described for Example 43 using N¹,N¹,N²-trinethylethane-1,2-diamine. Purification on a silica gel column using chloroformimethanol, (95:5), as the eluent gave 0.98 g (72% yield) of the, title compound as a yellow oil: ¹H NMR (CDCl₃, 400 MHz) δ 7.48 (d, J=8 Hz, 2H), 7.23 (d, J=9 Hz, 2 25s H), 3.59 (br s, 1H), 3.27 (br's, 1H), 3.03 (s, 1H), 2.94 (s, 2H), 2.52 (br s, 1H), 2.35 (br S, 1H), 2.26 (s, 3H), 2.04 (s, 3H); 130 NMR (CDCl₃, 100 MHz) δ 170.1, 135.3, 131.4, 128.6, 123.5, 57.2, 56.4, 49.3, 45.6; MS (EI) m/z 285 (M⁺+1).

### Example 45

### N-[2-Fluoro-4-((4-methyl-1-piperazinyl)sulfonyl]phenyl]acetamide

To solution of 4-(acetylamino)-3-fluorobenzenesulfonylfluoride (0.566 g, 2.4 mmol) in dry tetrahydrofuran (5 mL) was added N-meth piperazine (0.25 mL, 2.3 mmol) and triethylamine (0.52 mL, 3.6 mmol) at room temperature. The mixture was stirred at room temperature for 5 days and then heated to 60° C. for 2 days. The mixture was cooled to room temperature and a precipitation was formed. The precipitation was filtered and washed with cold methylene chloride and dried in vacuo to give 0.724 g (95% yield) of the title compound as a white solid: ¹H NMR (CD₃CN, 400 MHz) δ 8.45 (m, 2H), 7.48 (m, 2H), 2.96 (t, J=5 Hz, 4H), 2.38 (t, J=5 Hz, 2H), 2.17 (s, 3H), 2.16 (s, 3H); ¹³C NMR (CD₃CN, 100 MHz) δ 170.5, 153.9, 151.4, 132.4, 131.4, 131.4, 125.4, 122.5, 118.3, 15.8, 115.5,54.8, 47.0, 45.9, 24.6; MS (ESP) m/z 316 (M⁺+1).

### Example 46

### 2-Methyl-4-[(4-methylpiperazin-1-yl)sulfonyl]aniline

A suspension of 2-aminotoluene-5-sulfonic acid (10.1 g, 54 mmol) in thionyl chloride (80 mL) and N₃N-dimethylformanride (0.5 mL) was refluxed for, 28 h to give a dark solution. The solvent was evaporated and the resulting residue was suspended in tetrahydrofuran/methylene chloride (100:50 mL). 1-Methylpiperazine (25 mL, 225 mmol) was added carefully, and the resulting mixture was stirred at room temperature for 45 min. The solvent was evaporated and the crude product was purified by column chromatography using methylene chloride/methanol, (9:1), as the eluent to give 6.34 g (44% yield) of the title compound; ¹H NMR (CDCl₃, 400 MHz) δ 7.39 (m, 2H), 6.66 (m, 1H), 4.07 (s, 2H), 3.06 (br s, 4H), 2.58 (br s, 4H), 2.33 (s, 3H), 2.15 (s, 31); ¹³C NMR (CDCl₃, 100 MHz) δ 149.6, 130.6, 128.1, 123.3, 122.0, 114.2, 54.3, 45.9, 45.8, 17.7.

### Example 47

### 1-[(4-Bromo-3-methylphenyl)sulfonyl]-4-methylpiperazine

A solution of sodium nitrite (0.385 g, 5.58 mmol) in water (2 mL) was added dropwise to a stirred solution of 2-methyl-4-[(4-methylpiperazin-1-yl)sulfonyl]aniline (1.2 g, 4.45 mmol) in HBr (aq. conc. 17 mL) andwater (10 mL) at 5° C. The resulting mixture was stirred at 5° C. for 30 min and a solution of CuBr (0.332 g, 2.31 mmol) in HBr (aq. conc. 12 mL) was added. The resulting mixture was stirred at 5° C. for 20 min, and at 70° C. for 1 h. The reaction mixture was allowed to cool to room temperature and ice was added carefully to give an orange precipitate. The crystals were collected, washed with water and purified by column chromatography on silica using methylene chloride methanol, (9:1), as the eluent to give 0.62 g (42. % yield) of the title compound as white crystals; ¹H NMR (CDCl₃, 400 MHz) δ 7.68 (d, J=8 Hz, 1H), 7.58 (d, J=2 Hz, 1H), 7.40 (dd, J=8, 2 Hz, 1H), 3.10 (br s, 4H), 2.61 (br s, 4H), 2.46 (s, 3H), 2.33 (s, 3H); ¹³CNMR (CDCl₃, 100 MHz) δ 139.8, 134.6, 134.0, 130.8, 129.7, 126.6, 53.8, 45.5, 45.4, 23.3; MS (TSP) 333 and 335 (M⁺+1).

### Example 48

### 2-Fluoro-4-[(4-methyl-1-piperazinyl)sulfonyl]benzenamine

N-[2-Fluoro-4-[(4-methyl-1-piperazinyl)sulfonyl]phenylacetamide (0.724 g, 2.3 mmol) in HCl (30 mL, 18% in water) was heated at 110° C. for 30 min. The solution was cooled to 0-C and aqueous NaOH (conc. 46%) was added dropwise until the solution reached pH 5 and a precipitate was formed. The mixture was stirred at room temperature for 20 min then the precipitate was filtered and washed with cold water to give 0.484 g (75% yield) of the title compound as a off-white solid: ¹H NMR (CD₃CN, 400 MHz) δ 7.31 (m, 2H), 6.89 (m, 1H), 4.91 (br s, 2H), 3.01 (br s, 4H), 2.56 (br s, 2H), 2.29(s, 3H); ³CNMR (CD₃CN, 100 MHz) δ 150.5, 148, 140.6, 140.5, 125.1, 125.0, 121.9, 121.9, 117.0, 115.1, 115.0, 114.7, 114.5, 53.1, 45.0, 43.9; MS (ESP) m/z 272 and 274 (M⁺+1).

### Example 49

### 1-[(4-Bromo-3-fluorophenyl)sulfonyl]-4-methylpiperazine

To a solution of 2-fluoro-4-[(4-methyl-1-piperazinyl)sulfonyl]benzenamine (0.430 g, 1.57 mmol) in HBr (5 mL, 46%.in water) was added sodium nitrite (0.13 g, 1.89 mmol), in water (2 mL), dropwise at 0-5° C. After 30 min of stirring at 0-5° C., CuBr (75 mg, 0.52 25-mmol) in HBr (1 mL, 46% in water) was-added dropwise and the resulting mixture was stirred at 70° C. for 1 h. Cold water and ice was added and the solution was alkalyzed with saturated NaCO₃ (aq) and a precipitate was formed. The water mixture was partitioned between water and methylene chloride. The water phase was extracted with methylene chloride (3 times), the combined organic phases were dried (MgSO₄) and evaporated in, vacuo. The product was purified by column chromatography using methylene chloride/methanol, (95:5), as the eluent to give 0.256 g (48% yield) of the title compound as a beige colored solid: ¹H NMR (CD₃CN, 400 MHz) δ 7.86 (m, 1H), 7.57 (m, 1H), 7.47 (m, 1H), 2.99 (t, J=5 Hz, 4H) 2.38 (t, J.=5 Hz, 4H), 2.18 (s, 3H); ¹³C NMR (CD₃CN, 100 MHz) 159.8, 157.3, 136.9, 136.9, 134.4, 124.4, 124.4, 117.0, 115.6, 115.4, 114.1, 113.9, 53.4, 45.7, 44.5; MS (ESP) m/z 339 (M⁺+1).

### Example 50

### 4-[(4-Methylpiperazin-1-yl)sulfonyl]-2-(trifluoromethyl)aniline

Chlorosulfonic acid (6.5 mL, 96 mmol) was added to 2-(trifluoromethyl)aniline (5.0 mL, 40 mmol) under stirring to give a solid that was slowly dissolved upon heating. The mixture was heated at 60° C. for 2 h, allowed to cool to room temperature and was poured over ice to give a white solid. The solid was filtered off, dissolved in tetrahydrofuran (30 mL) and 1-methylpiperazine (4.5 mL, 41 mmol) was added. The resulting mixture was stirred at room temperature for 20 min, and the solvent was evaporated to give the crude product. Purification by column chromatography on silica using methylene chloride/methanol, (9:1), as the eluent gave 0.414 g (3% yield) of the title compound as white crystals: ¹H NMR (CDCl₃, 400 MHz5) δ 7.79 (m, 1H), 7.62 (m, 1H), 6.78 (d, J=9 Hz, I H), 4.68 (br s, 2H), 3.04 (r s, 4H), 2.52 (br s, 4H), 2.30 (s, 3H).

### Example 51

### 1-[(4-Bromo-3-(trifluoromethyl)phenyl]sulfonyl)₄-methylpiperazine

The title compound was prepared as described for Example 47 using 4-[(4-methylpiperazin--yl)sulfonyl]-2-(trifluoromethyl)aniline: yield 32%; ¹H NMR (CDCl₃, 400 MHz) δ 8.02 (d, J=2 Hz, 1H), 7.90 (d, J=8 Hz, 1H), 7.74 (dd, J=8, 2 Hz, 1H), 3.13 (br s, 4H), 2.57 (br s, 4H), 2.34 (s, 3H); ¹³C NMR (CDCl₃, 100 MHz) δ 136.1, 135.6, 131.6, 126.9, 123.4, 120.7, 53.7,45.2,45.1; MS (ES) 387 and 389 (M⁺+1).

### Example 52′

### 1-[(4-Bromo-2-fluoro-5-methylphenyl)sulfonyl]4-methylpiperazine

2-Bromo-4-fluoro-1-methylbenzene (1.5 g, 7.9 mmol) was cooled to 0° C. and chlorosulfonic acid (1.85 g, 15.9 mmol) was slowly added. The reaction mixture was allowed to warm to room temperature after 10 min and stirring was continued for 30 min. The reaction mixture was then warmed to 80° C. and stirred for 3 h. The reaction mixture was cooled to room temperature and slowly added to an ice/water mixture. The precipitate was dissolved in a methylene chloride/tetrahydrofuran mixture, (10:1, 60 mL), and washed with a saturated sodium hydrogen carbonate solution. The organic layer was dried over magnesium sulfate. Filtration and removal of the solvent in vacuo yielded 1.3 g of the crude sulfonchloride that was dissolved in tetrahydrofuran (20 mL) and cooled to 0° C. N-Methylpiperazine (2 mL) was added and stirring was continued for 30 min at room temperature. A saturate aqueous sodium hydrogencarbonate solution (20 n L) was added and the mixture was extracted with methylene chloride. The organic layer was dried over sodium sulfate. Filtration and removal of the solvent in vacuo gave a residue which was purified-by chromatography on silica gel using a gradient ethyl acetate to ethyl “acetate/methanol, (1:1), as the eluent to give 1.09 g (39% yield) of the title compound: ¹H NMR (DMSOd6, 400 MHz) δ 7.86 (d, J=10 Hz, 1H), 7.72 (d, J=8 Hz, 1H), 3.04 (m, 4H), 2.38 (s, 3H), 2.34 (m, 4H), 2.14 (s, 3H); MS (ES) m/z 352 (M⁺+1).

### Exampe 53

### 1-[(4-Bromo-2,5-dimethylphenyl)sulfonyl]-4-methylpiperazine

The title compound was prepared as described for Example 52 using 2-bromo-1,4-dimethylbenzene, yield 32%: ¹H NMR (DMSO-d₆, 400 MHz) δ 7.71 (s, 2H), 3.02 (m, 4 H), 2.48 (s, 3H), 2.37 (s, 3H), 2.32 (m, 4H), 2.14 (s, 3H).

### Example 54

### 1-[(4-Bromophenyl)sulfonyl]piperidine

Piperidine (3.0 g, 35.2 mmol) was added to a solution of 4-bromo-benzenesulfonyl chloride 4.5 g, 17.6 mmol) in methylene chloride (10 mL) at 0° C. The mixture was stirred for 2 h, NaOH (aq) (1 M, 5 mL) was added and stirring was continued for 10 min. The organic phase was separated and diluted with methylene chloride (40 mL), washed with HCl (aq) (1 M, 10 mL) and water. The organic phase was dried (sodium sulfate) and the solvent was evaporated to give 5.1 g (96% yield) of the title compound as a white solid: ¹³C NMR. (solvent, 100 MHz) δ 35.33, 132.16, 129.05, 127.48, 46.82, 25.04, 23.34; MS (E-S) m/z 304 and 306 (M⁺+1). The following Examples, 55-57, were synthesized as described for Example 54: Example 55 1-[(4-Bromophenyl)sulfonyl]pyrrolidine

Starting materials: pyrrolidine and 4-bromobenzenesulfonyl chloride. Yield 98% as a white solid: ¹³C NMR (solvent, 100 MHz) δ 135.93, 132.17, 128.84, 127.39,47.84,25.13; MS (ES) m/z 290 and 292 (M⁺+1).

### Example 56

1-[(4-Bromo-2,5-difluorophenyl)sulfonyl]piperidine

Starting materials: piperidine and 4-bromo-2,5-difluorobenzenesulfonyl. Yield 96% as a white solid: MS (ES) m/z 340 and 342 (M⁺+1).

### Example 57

### 1-[(4-Bromo-2,5-difluorophenyl)sulfonyl]pyrrolidine

Starting materials: pyrrolidine and 4-bromo-2,5-difluorobenzenesulfonyl chloride, yield 97%: MS (ES).m/z 326 and 328 (M⁺+1).

### Example 58

### tert-Butyl 4-[(4-bromophenyl)sulfonyl]piperazine-1-carboxylate

4-Dimethylaminopyridine (16 mg, 0.13 mmol) and di-tert-butyl dicarbonate (0.317 g, 1.45 mmol) was added to a stirred solution of 1-[(4-bromophenyl)sulfonyl]piperazine (0.40 g, 1.31 mmol) in tetrahydrofuran (12 mL) and the resulting mixture was stirred at room temperature for 30 min. The solvent was evaporated and the crude product purified by chromatography on silica gel using heptanelethyl acetate, (2:1), as the eluent to give 0.506 g (95% yield) of the title compound: ¹H NMR (CDCl₃, 400 MHz) δ 7.70 (m, 2H), 7.61 (m, 2H), 3.52 (t, J=5 Hz, 4H), 2.98 (t, J=5 Hz, 4H), 1.42 (s, 9H); ¹³CNMR (CDCl₃, 100 MHz) δ 154.3, 134.7, 132.7, 129.4, 128.4, 80.7, 46.0, 28.5.

### Example 59

### 1-(4-Bromobenzoyl)-4-methylpiperazine

4-Bromobenzoic acid (3.0 g, 14.9 mmol) was dissolved in refluxing thionyl chloride (35 mL) and the solution was heated under reflux for 1 h and then cooled to room temperature. The solvent was evaporated, co-evaporated with toluene (3×40 mL), and the resulting solid was dried in vacuo. The solid was dissolved in methylene chloride (118 mL), cooled on ice-bath, and 1-methylpiperazine (1.5 mL, 13.6 mmol) was added dropwise to give a solid. Methylene chloride/K₂CO₃ (saturated, aq.) was added and the aqueous phase was extracted with methylene chloride. The combined organic phases were dried over Na₂SO₄, filtered, and the solvent was evaporated to give 3.86 g (91% yield) of the title compound: ¹H NMR (DMSQ-d6, 300 MHz) δ 7.64 (d, J=8 Hz, 2H), 7.34 (d, J=8 Hz, 2H), 3.59 (m, 4H), 2.34 (m, 4 H), 2.21 (s, 3H); MS (ES) 283 and 285 (M⁺+1).

### Example 60

### 3-(4-Bromophenoxy)-1-methylpyrrolidine

A mixture of p-bromophenol (0.5 g, 2.89 mmol), 1-methyl-3-pyrrolidionol(0.38 ml, 3.47 mmol) and triphenylphosphine (0.91 g, 3.47 mmol) was dissolved in anhydrous tetrahydrofuran (8 mL) and cooled to 0° C. Diethyl azodicarboxylate (0.55 ml, 3.47 mmol) was added dropwise and the resulting mixture was stirred at room temperature overnight. The solvent was evaporated and the residue partioned between water and ethyl acetate. The organic phase was washed twice with water, dried (MgSO₄) and the solvent was evaporated. The product was purified by column chromatography on silica using methylene chloride/methanol, (98:2), as the eluent to give the title compound as a clear oil which crystallized on standing, yield 77%: ¹H NMR (IMSO-d6, 400 MHz) δ 7.36 (d, J=9 Hz, 2H), 6.80 (d, J=9 Hz, 2H), 4.83 (m, 1H), 2.73 (m, 1H), 2,64 (m, 1H), 2.59 (m, 1H), 2.34 (m, 2H), 2.25 (s, 3H), 1.73 (m, 1H); ¹³C NMR (DMSO-d₆, 100 MHz) δ 165.6, 132.1, 117.3, 111.7, 76.9, 61.6, 54.5, 41.6, 32.2; MS (ESP) nmz 258 (M⁺+1).

### Example 61

### tert-Butyl 4-[2-(4-bromopherioxy)ethyl]piperazine-1-carboxylate

Diethyl azodicarboxylate (1.72 mL, 10.9 mmol) was added dropwise to a cooled (0° C.) solution of tert-butyl 4-(2-hydroxyethyl)piperazine-1-carboxylate (2.10 g, 9.1 mmol; described in: Xue, C. B. Bioorg. Med. Chem. 1997, 5, 693.), 4-bromopbenol (1.58 g, 9.1 mmo]), and triphenylphosphine (3.10 g, 11.9 mmol) in tetrahydrofuran (30 mL). The resulting mixture was stirred at room-temperature for 23 h and the solvent was evaporated. Purification by chromatography on silica using methylene chloride/methanol/triethylamine, (95:5:0.1), as the eluent gave 0.50 g (14% yield) of the title compound: ¹H NMR (DMSO-d6, 300 MHz) δ 7.43 (m, 2H), 6.92 (m, 2H), 4.06 (t, J=6 Hz, 2H), 3.30 (t, J=5 Hz, 4H), 2.69 (t, J=6 Hz, 2H), 2.42 (t, J=5 Hz, 4H), 1.39 (s, 9H).

The following Examples, 62-65, were synthesized as described for Example 61:

### Example 62

### tert-Butyl 4-[2-(4-bromo-2,5-difluorophenoxy)ethyl]piperazine-1-carboxylate

Starting material: 4-bromo-2,5-difluorophenol, yield 62%: ¹H NMR (DMSO-d₆, 300 MHz) δ 7.67 (dd, J=11, 7 Hz, 1H), 7.38 (dd, J=10, 8 Hz, 1H), 4.18 (t, J=6 Hz, 2H), 3.30 (m, 4-H), 2.73 (t, J=6 Hz, 2H), 2.43 (m, 4H), 1.39 (s, 9H); MS (ES) 421 and 423 (M⁺+1).

### Example 63

### 4-[2-(4-Bromo-2,5-difluorophenoxy)ethyl]morpholine

Starting material: 4-bromo-2,5-difluorophenol and 4-(2-hydroxyethyl)morpholine, yield 55%: ¹H NMR (DMSO-d₆, 300 MHz) S.7.68 (dd, J=11, 7 Hz, 1H), 7.39 (dd, J.=11, 8 Hz, 1H), 4.18 (t, J=6 Hz, 2H), 3.56 (t,J=5 Hz, 2H), 2.70 (t, J=6 Hz, 2H), 2.46 (t, J=5 Hz, 4H), 1.18 (m, 2H).

### Example 64

### 1-[2-(4-Bromo-3,5-dimethylphenoxy)ethyl]-4-tmethylpiperazine

Starting material: 2-(4-methylpiperazin-1-yl)ethanol (described in: Ide, W. S. et al, J. Am. Chem. Soc. 1954, 76, 1122) and 4-bromo-3,5-dimethylphenol, yield 64%: ¹H NMR (DMSO-d₆, 300 MHz) δ 6.80 (s. 2H), 4.02 (t, J=6 Hz, 2H), 2.65 (t, J=6 Hz, 2H), 2.46 (m, 4H), 2.31 (m, 10H), 2.14 (m,+3H).

### Example 65

### 1-[2-(4-Bromo-3-methylphenoxy)ethyl]-4-methylpiperazine

Starting material: 2-(4-methylpiperazin-1-yl)ethanol (described in: Ide, W. S. et al, J. Am. Chem. Soc. 1954, 76,1122) and 4-bromo-3-methylphenol, yield 83%: ¹H NMR (DMSO-d6, 300 MHz)37.42(d, J=9 Hz, 1H), 6.97 (d, J=3 Hz, 1H), 6.72 (dd, =9,3 Hz, 1H), 4.03 (t, J=6 Hz, 2H), 2.65 (t, J=6 Hz, 2H), 2.46 (m, 4H), 2.29 (m, 7H), 2.14 (s, 3H); MS (ES) 313 and 315 (M⁺+1).

### Example 66

### 1-[2-(4-Bromo-2,5-difluorophenoxy)ethyl]pyrrolidine

A solution of 4-bromo-2,5-difluorophenol (0.36 g, 1.7 mmol), 1-(2-chloroetbyl)pyrrolidine hydrochloride (0.38 g, 2.2 mmol), and potassium carbonate (0.86 g, 6.2 mmol) in N,N-dimethylformamide (10 mL) was stirred at 80° C. for 16 h. The solution was cooled to room temperature, water was added and the aqueous-phase was extracted with methylene chloride. The combined organic phases were evaporated, co-evaporated with toluene (4×30 mL), and the resulting solid was dried under vacuum to give 0.51 g (97% yield) of the title compound: ¹H NMR (DMSO-d₆, 300 MHz) δ 7.68 (dd, J=11, 7 Hz, 1H), 7.37 (dd, J=10, 8 Hz, 1H), 4.15 (t, J=6 Hz, 2H), 2.79 (t, J=6-Hz, 2H), 2.50 (m, 4H), 1.67 (m, 4H).

### Example 67

### 5-Bromo-N,N-dimethylthiophene-2-sulfonamide

5-Bromothiophene-2-sulfonyl chloride (1 g, 3.8 mmol) was dissolved in tetrahydrofuran (20 mL) and the solution was cooled to 0° C. Dimethylamine (8 mL, 2 M in ethanol, 16 mmol) was added and stirring was continued for 20 min. The reaction mixture was allowed to warm to room temperature and water (20 mL) and ethyl acetate (40 mL) were added. The layers were separated and the aqueous layer was extracted with ethyl acetate. The combined organic layers were dried over magnesium sulfate. Filtration and removal of the solvent in vacuo gave a residue which was purified by column chromatography on silica using a gradient heptane/ethyl acetate (100:0->0: 100)-to give 1 g (97% yield) of the title compound as a solid: ¹H NMR (CDCl₃, 400 MHz) δ 7.31 (d, J=4 Hz, 1H), 7.15 (d, J=4 Hz, 1H), 2.77 (s, 6H); MS (ES) m/z 270 and 272 (M⁺+1).

### Example 68

### tert-Butyl 4-(S-bromo-2-furoyl)piperazine-1-carboxylate

1-(2-Furoyl)piperazine (2 g, 1.1 mmol) and sodium acetate (1.8 g, 22 mmol) were dissolved in acetic acid (40 mL, 0.7 mmol). Bromine was added dropwise and the solution was stirred for 12 h. The solution was poured on ice (300 mL) and the aqueous solution was neutralized with solid sodium carbonate. The aqueous solution was extracted with chloroform and the combined organic layers were dried over magnesium sulfate. Filtration and removal of solvent in vacuo-gave a residue, which was dissolved in tetrahydrofuran (10 mL). Di-tert-butyldicarbonate (2.6 g, 12 mmol) was added and the reaction mixture was stirred for 30 min at room temperature. The solvent was removed in vacuo and the residue was purified by column chromatography on silica gel using a gradient ethyl acetate/heptane (1:100->0:100) as the eluent to give 263 mg (7% yield) of the title compound as a white solid: ¹H NMR (DMSO-d₆, 400 MIz) δ 6.82 (m, 1H), 6.24 (m, 1H), 3.61 (m, 4H), 3.34 (m, 4H), 1.31 (s, 9H); MS (ES) m/z 359 and 361 (M⁺+1).

The following Examples 69-71 were synthesized as described for Example 7:

### Example 69

### 3-Ethyl-4-(4-methylpiperazin-1-yl)sulfonyl]phenylboronic acid

Starting material: 1-[(4-bromo-2-ethylphenyl)sulfonyl)₄-methylpiperazine, yield 55%: ¹HNMR (CD₃OD, 400 MHz) δ 7.78 (d, J=8 Hz, 1H), 7.73 (s, 1H), 7.63 (d, J=8 Hz, 1H), 3.22 (m, 4H), 3.01 (q, J=8 Hz, 2H), 2.66 (m, 4H), 2.40 (s, 3H), 1.27 (t, J=8 Hz, 3H); ¹³C NMP (CD₃OD, 100 MHz) δ 144.3, 138.1, 136.1, 132.3, 130.1, 55.2, 45.8, 45.6, 27.6, 16.9.

### Example 70

### 4[(4-Methylpiperazirin-1-yl)sulfonyl]-3-(trifluoromethoxy)phenylboronic acid

Starting material:1-{[4-bromo-2-(trifluoromethoxy)phenyl]sulfonyl}-4-methylpiperazine, yield 61%: ¹HNMR (CD₃OD, 400 MHz) δ 7.73 (d, J=8 Hz, 1H), 7.62 (m, 2H), 3.19 (m, 4H), 2.47 (m, 4. H), 2.26 (s, 3H); ³C NMR (CD₃OD, 100 MHz) δ 146.6, 132.7,130.7; 126.7, 126.3, 125.8, 123.3, 120.7, 55.4, 46.7, 46.0; MS (TSP) n-z 369 (M⁺+1).

### Example 71

### 4-{[4-(tert-Butoxycarbonyl)piperazin-1-yl]sulfonyl}phenylboronic acid

Starting material: tert-butyl 4-[(4-bromophenyl)sulfonyl]piperazine-1-carboxylate, yield 94%: ¹H NMR (CD₃OD, 400 MHz) δ 7.93 (m, 2H), 7.14 (m, 2H), 3.49 (br s, 4H), 2.95 (br s, 4H), 1.40 (s, 9H); ¹³CNMR (CD₃OD, 400 MHz) δ 156.1, 135.6, 81.9, 47.3, 28.6.

### Example 72

### 2,5-Difluoro-4-(piperidin-1-ylsulfonyl)phenylboronic acid

n-Butyllitium (13 mL, 22.1 mmol) was added dropwise over 30 min to a cooled (−78° C.) solution of 1-[(4-bromo-2,5-difluorophenyl)sulfonylpiperidine (2.5 g, 7.35 mmol) and triisopropyl borate (4.5 g, 22.1 μmol) in anhydrous tetrahydrofuran (15 mL) under nitrogen atmosphere. The reaction mixture was stirred for 12 h while the temperature was allowed to reach room temperature. HCl (aq) (5 mL, 2 M) was added and stirring was continued for 30 min. Additional methylene chloride (100 mL) was added and the organic phase was washed with HCl (aq) (20 mL, 2 M). The organic phase was dried (sodium sulfate) and evaporated. The remaining residue was purified by reversed phase chromatography (C-18) using a gradient water/acetonitrile to give 1.2 g (53% yield) of the title compound: ¹H NMR (CD₃OD, 400 MHz) δ 7.41 (dd, J=10, 5 Hz, 1H), 7.37 (dd, J 4, 4 Hz, 1H), 3.12 (m, 4H), 1.58 (m, 4H), 1,47 (m, 2H); MS (ES) m/z 306 (M⁺+1)

The following Examples, 73-76, were synthesized as described for Example 72:

### Example 73

### 2,5-Difluoro-4-(pyrrolidin-1-ylsulfonyl)phenylboronic acid

Starting material: 1-[(4bromo-2,5-difluorophenyl)sulfonyl]pyrrolidine. Yield 48% ¹H NMR (CD₃OD/CDCl₃, (1:1), 400 MHz) δ 6.68 (d, J=8 Hz, 1H), 6.23 (dd, J=2, 2 Hz, I H), 2.50 (m, 4H), 1.31 (m, 4H); MS (ES) m/z 292 (M⁺+1).

### Example 74

### 4-(Pyrrolidin-1-ylsulfonyl)phenylboronic acid

Starting material: 1-[(4-bromophenyl)sulfonyl]pyrrolidine. Purification on a silica gel column using a gradient of methylene chloride to methylene chloride/ethanol, (1:1), gave the title compound as a white solid, yield 70%: ¹³C NMR (CDCl₃/CD₃OD (1:1), 100 MHz) δ 136.79, 133.50, 125.4s, 47.19, 24.30; MS (ES) “/z 256 (M⁺+1).

### Example 75

### 4-(Piperidin-1-ylsulfonyl)phenylboronic acid

Starting material: 1-[(4-bromophenyl)sulfonyl]piperidine, Yield 78% asia white solid: ³C NMR (CDCl₃₁CD₃OD (1:1), 100 MHz) δ 136.35,133.56,125.84, 46.39, 24.87, 24.52, 22.76; MS (ES).m/z 270 (M⁺+1).

### Example 76

### 4-[(Dimethylamino)sulfonyl]phenylboronic acid

Starting material: 4-bromo-N,N-dimethylbenzenesulfonamide. Purification by chromatography on a silica gel column using a gradient methylene chloride to methylene chloride/methanol, (2:1), as the eluent gave the title compound, yield 60%: MS (ES) m/z 230 (M⁺+1).

### Example 77

### 4-((Methyl(-1-methylpyrrolidin-3-yl)amino)sulfonyl)phenylboronic acid

To a solution of 4-bromo-N-methyl-N-(1-methylpyrroidin-3-yl)benzenesulfonamide (333 mg, 1 mmol) and triisopropyl borate (1146 ul, 5 mmol) in tetrahydrofuran (7 ML) was added n-butyllithitim (2 mL, 2.5 M solution in hexaney slowly at −78° C. The mixture was stirred at −78° C. for 16 h and then heated to room temperature. 2 mL of water was added, and the mixture was stirred for another 30 min. A two-phase system has formed, where the light phase was discarded. 1 g of celite was added to the aqueous phase and the solvent was removed by evaporation. Chromnatography on silica using a gradient of methylen chloride (d00%) to methanol (100%) followed by methanol (100%) to methanol/water, (1:1), afforded 300 mg of the title compound after removal of the solvents: MS (ES) m/z 299, (M⁺+1)

The following Examples 78-80 were synthesized as described for Example 77:

### Example 78

### 4-((4-Acetylpiperazin-1-yl)sulfonyl)phenylboronic acid

Starting material: 1-acetyl-4-[(4-bromophenyl)sulfonyl]piperazine: MS (ES) m/z 313 (M⁺+1).

### Example 79

### 4-(((2-Dimethylamino)ethyl)(ethyl)amino)sulfonyl)phenylboronic acid

Starting mlaterial: 4-bromo-N-[2-(dimethylamino)ethyl]-N-ethylbenzenesulfonamide: MS (ES) m/z 301 (M⁺+1).

### Example 80

### 4-((3-Dimethylamino)pyrrolidin-1-yl)sulfonyl)phenylboronic acid

Starting material: 1-[(4-bromophenyl)sulfonyl]-NN-dimethylpyrrolidin-3-amine: MS (ES) m/z 299 (M⁺+1).

### Example 81

### 4-(((2-Dimethylamino)-1-methylethyl)amino)sulfonyl)phenylboronic acid.

To a solution of 4-bromo-N-[2-(dimetbylamino)-1-methylethyl)benzenesulfonamide (286 mg, 1 mmol) and triisopropyl borate (1146 uL, 5 mmol) in tetrahydrofuran (7 mL) was added n-buthyl lithium (2 mL, 2.5 M solution in hexane) slowly at −78° C. The mixture was stirred at −78° C. for 16 h and then heated to room temperature. Water (2 mL) was added, and the mixture was stirred for another 30 min. A two-phase system has formed, where the light phase was discarded. Celite (1 g) was added to the aqueous phase and the solvent was removed by evaporation. The celite was packed in a reservoir on top of 5 g of C-18 silica, and eluted with 40 mL of water followed by evaporation in vacuo: MS (ES) mnz 287 (M++I).

The following Examples 82-96 were synthesized as described for Example 81:

### Example 82

### 4-((3-Pyrrolidin-1-ylpropyl)amino)sulfonyl)phenylboronic acid

Starting material: 4-bromo-N-(3-pyrrolidin-1-ylpropyl)benzenesulfonamide: MS (ES) m/z 313 (M⁺+1).

### Example 83

### 4-((Methyl-(1-methylpiperidin-4-yl)amino)sulfonyl)phenylboronic acid

Starting material: 4-bromo-N-methyl-N-(1-methylpiperidin-4-yl)benzenresulfonamide: MS (ES) m/z 313 (M⁺+1).

### Example 84

### 4-(((Dimethylamino)propyl)(methyl)amino)sulfonyl)phenylboronic acid

Starting material: 4-bromo-N-[3-(dimethylamino)propyl]-N-methylbenzenesulfonamide: MS (ES) m/z 301 (M⁺+1).

### Example 35

### 4-(Morpholin-4-ylsulfonyl)phenylboronic acid

Starting material: 4-[(4bromophenyl)sulfonyl]morpholine: MS (ES) m/z 342 (M⁺+1).

### Example 86

### 4-(((3-(4-Methylpiperazin-1-yl)propyl)amino)sulfonyl)phenylboronic acid

Starting material: 4-bromo-N-[3-(4-methylpiperazin-1-yl)propyl]benzenesulfonamide: MS (ES) m/z 342 (M⁺+1).

### Example 87

### 4-((4-Ethylpiperazin-1-yl)sulfonyl)phenylboronic acid

Starting material: 1-[(4-bromophenyl)sulfonyl]4-ethylpiperazine: MS (ES) m/z 299 (M⁺+1).

### Example 88

### 4-((2-Pyrrolidin-1-ylethyl)amino)sulfonyl)phenylboronic acid

Starting material: 4-bromo-N-(2-pyrrolidin-1-ylethyl)benzenesulfonamide: MS (ES) m/z 299 (M⁺+1).

### Example 89

### 4-((4-Methyl-1,4-diazepan-1-yl)sulfonyl)phenylboronic acid

Starting material: 1-[(4-bromophenyl)sulfonyl]-4-methyl-1,4-diazepane: MS (ES) m/z 299

### Example 90

### 4-(((2-Dimethylamino)propyl)amino)sulfonyl)phenylboronic acid

Starting material: 4-bromo-N-[2-(dimethylamino)propyl]benzenesulfonamide: MS (ES) m/z 287 (M⁺+1).

### Example 91

### 4-((Isopropyl-(2-methoxyethyl)amino)sulfonyl)phenylboronic acid

Starting material: 4-bromo-N-isopropyl-N-(2-methoxyethyl)benzenesulfonamide: MS (ES) m/z302 (M⁺+1).

### Example 92

### 4-((((1-Ethylpyrrolidin-2-yl)amino)sulfonyl)phenylboronic acid

Starting material: 4-bromo-N-[(1-ethylpyrrolidin-2-yl)methyl]benzenesulfonamide: MS (ES) m/z 313 (M⁺+1).

### Example 93

### 4-(((2-Diethylamino)ethyl)amino)sulfonyl)phenylboronic acid.

Starting material: 4-bromo-N-[2-(diethylamino)ethyl]benzenesulfonamide: MS (ES) m/z 301 (M⁺+1).

### Example 94

### 4-(((2-Pyridin-2-ylethyl)amino)sulfonyl)phenylboronic acid

Starting material: 4-bromo-N-(2-pyridin-2-ylethyl)benzenesulfonamide: MS (ES) m/z 307

### Example 95

### 4-(((2-Methoxy-1-methylethyl)amino)sulfonyl)phenylboronic acid

Starting material: 4-bromo-N-(2-methoxy-1-methylethyl)benzenesulfonamide: MS (ES) m/z 274 (M⁺+1).

### Example 96

### 4-(((3-Dimethylamino)propyl)amino)sulfonyl)phenylboronic acid

Starting material: 4-bromo-N-[3-(dimethylamino)propyl]benzenesulfonamide: MS (ES) m/z 287 (M⁺+1).

### Example 97

### tert-Butyl 4-[(dimethylamino)methyl]pyridin-3-ylcarbamate

tert-Butyl 4-formylpyridin-3-y]carbamate (0.10 g, 0.45 mmol) and dimethyl ammonium hydrochloride was mixed in-methylene chloride (2 mL) and stirred for 30 min; Sodium triacetoxyborohydride (0.19 g, 0.90 mmol)+was added and the resulting mixture was stirred for 1 h. The crude product mixture was pre-adsorbed onto silica and purified by chromatography on silica gel using gradient heptane to he ptane/ethyl acetate, (1:1), as the eluent to give 53 mg (47% yield) of the title compound as a oil: MS (ES) m/z 252 (M⁺+1).

### Example 98

### 4-[(Dimethylamino)methyl]pyridin-3-amine

Trifluoroacetic acid, 50% in methylene chloride (10 mL), was added to tert-butyl 4-[(dimethylamino)methyl)pyridin-3-ylcarbamate (0.20 g, 0.796 mmol). The reaction mixture was stirred for 2 h. The solvent was evaporated and the crude product was dissolved in water (5 mL) and freeze-dried to give 0.115 g (95% yield) of the title compound as a brown oil: ¹H NMR (CDCl₃, 400 MHz) δ 7.85 (s, 1H), 7.67 (d, J=5 Hz, 1H), 6.93 (d, J=5 Hz, 1H), 3.33 (s, 2H), 2.12 (s, 6H); MS (ES) m/z 152 (M++I).

### Example 99

### 4-(Pyrrolidin-1-ylmethyl)pyridin-3-amine

tert-Butyl 4-(pyrrolidin-1-ylmethyl)pyridin-3-ylcarbamate (1 g, 3.6 mmol) was dissolved in methylene chloride (20 mL) and trifluoroacetic acid (3 mL, 39 mmol) was added and stirring was continued for 30 min. The solvent was removed in vacuo and ethyl acetate (5 mL) were added and removed in vacuo. This procedure was repeated 3 times. The residue, was dissolved in methanol (50 mL) and DOWEX-OH was added until the m-ethanolic solution was basic. Filtration and removal of the solvent in vacuo gave the title 0.57 g (90% yield) of the title compound: ¹H NMR (CD₃OD, 400 MHz) δ 7.92 (s, 1H), 7.75 (d, J=5 Hz, 1H), 7.05 (d, J=5 Hz, 1H), 3.61 (s, 2H), 2.49 (m, 4H), 1.79 (m, 4H); MS (ES) m/z 178 (M⁺+1).

The following Examples, 100-101, were synthesized as described for Example 99:

### Example 100—

### 4-(2-Pyrrolidin-1-ylethyl)pyridin-3-amine

Starting material: tert-butyl 4-(2-pyrrolidin-1-ylethyl)pyridin-3 ylcarbamate, yield 80%: ¹H NMR (CD₃OD, 400 MHz) δ 7.95 (s, 1H), 7.75 (d, J=5 Hz, 1H), 7.04 (d, J=5 Hz, I—H), 2.75 (m, 4H), 2.66 (m, 4H), 1.86 (m, 4H); MS (FS) n7/z 192 (M⁺+1).

### Example 101

### 4-(3-Pyrrolidin-1-ylpropyl)pyridin-3-amine

Starting material: tert-butyl 4-(3-pyrrolidin-1-ylpropyl)pyridin-3-ylcarbamate, yield 80%: ¹H NMR (CD₃OD, 400 MHz)δ 7.91 (s, 1H), 7.72 (d, J=6 Hz, 1H), 7.02 (d, J=5 Hz, 1-H, 2.59-2.49 (m, 8H), 1.87-1.79 (m, 6H); MS (ES) m/z 206 (M⁺+1).

### Example 102

### tert-Butyl 4-(pyrrolidin-1-ylmethyl)pyridin-3-ylcarbamate

Tert-Butyl 4-formylpyridin-3-ylcarbamate (1.03 g, 4.64 mmol; described in: Venuti, M. C. et al. J. Med. Cliem. 1988, 31, 2136-2145) was dissolved in 11,2-dichloroethane (20 mL) under nitrogen atmosphere. Pyrrolidine (0.41 mL, 4.9 mmol) and acetic acid (0.27 mL) 4.72 mmol) were added and the reaction mixture was stirred for 1 h. Sodium-triacetoxyborobydride (1.27 g, 6 mmol) was added and stirring was continued for 10 h. Sodium hydroxide solution (1 M, 5 ml, 5 mmol) was Added and the layers were separated. The aqueous layer wasp extracted with methylene chloride and the combined organic layers were dried over sodium sulfate. Filtration and removal of the solvent in vacuo yielded a residue. Purification on a silica gel column using a radient methylene chloride/methanol, (100:2) to (100:10), as the eluent gave 900 mg (70% yield) of the title compound as an oil: ¹H NMR (CDCl₃, 400 MHz) δ 9.83 (s, 1H), 9.21 (s, 1H), 8.16 (d, J=5 Hz, 1H), 6.96 (d, J=5 Hz, 1H), 3.66 (s, 2H), 2.49 (m, 4H), 1.81 (m, 4H), 1.52 (s, 9H); MS (ES) m/z 278

### Example 103

### tert-Butyl 4-(2-pyrrolidin-1-ylethyl)pyridin-3-ylcarbamate

tert-Butyl 4-(2-hydroxyethyl)pyridin-3-ylcarbamate (1 g, 4.2 mmol) was dissolved in methylene chloride (40 mL) under inert gas atmosphere and cooled to 0° C. Methanesulfonyl chloride (0.48 mL, 6.3 mmol) and triethylamine (1.8 mL, 12.6 mmol) were added and stirring was continued for 1.5 h. Pyrrolidine (1.76 mL, 21 mmol) was added and the reaction mixture was stirred for 12 h at room temperature. Saturated aqueous sodium chloride solution (5 mL) was added and the organic layer was separated and dried over sodium sulfate. Filtration and removal of the solvent in vacuo yielded a residue, which was purified by chromatography on silica gel using ethyl acetate/heptane, (1:8->1:1), as the eluent to give 730 mg (60% yield) of the title compound as an oil: ¹H NMR (CDCl₃, 400 MHz) δ 9.09 (br s, 1H), 8.18 (d, J=5 Hz, 1H), 6.96 (d, J=5 Hz, 1H), 2.76 (m, 4H), 2.66 (m, 4H), 1.89 (m, 4H), 1.54 (s, 9H); MS (ES) m/z 292 (M⁺+1).

### Example 104

### tert-Butyl 4-(2-hydroxyethyl)pyridin-3-ylcarbamate

Tert-Butyl pyridin-3-ylcarbamate (2 g, 10.3 mmol, described in Kelly, T. A., McNiel, D. W., Tetrahedron Lett. 1994, 35, 9003-9006) was dissolved under inert gas atmosphere in tetrahydrofuran (60 mL) and the solution was cooled to −78° C. Tert-butyl lithium (14 mL, 1.7 M in pentane) was added dropwise and stirring was continued for 3 h. Ethylene oxide (1 mL, 20 mmol) was added dropwise and the reaction was allowed to warm up to room temperature. Saturated ammonium chloride solution was added (5 mL). The organic layer was separated and dried over magnesium sulfate. Filtrationand.removal of the solvent in vacuo yielded a residue which was purified by column chromatography on silica gel using heptane/ethyl acetate, (10:1->0:100), as the eluent to give 1.7 g (70% yield) of the title compound as a white solid: ¹H NMR (CD₃OD, 400 MHz) δ 8.66 (s, 1H), 8.22 (d, J=5 Hz, 1H), 7.33 (d, J=5 Hz, 1H), 3.83 (t, J=6 Hz, 2H), 2.89 (t, J=7 Hz, 2H), 1.54 (s 9H); MS (ES) m/z 239 (M⁺+1).

### Example 105

### tert-Butyl 4-(3-pyrrolidin-1-ylpropyl)pyridin-3-ylcarbamate

tert-Butyl 4-(3-pyrrolidin-1-ylprop-1-ynyl)pyridin-3-ylcarbamate (1.23 g, 4 mmol) was dissolved in 20 mL methanol. Palladium (10%) on charcoal (40 mg) was added and the reaction mixture was shaken for 12 h under hydrogen pressure (40 psi). The reaction mixture was filtered through a pad of celite and the solvent was removed in vacuo to give 1.2 g (97% yield) of title compound as an oil: ¹H NMR (CDCl₃, 400 MHz) δ 10.17 (br s, 1H), 8.91 (br s, 1H), 8.22 (d, J=5 Hz, 1H), 7.03 (d, J=5 Hz, 1H), 2.70 (t, J=6 Hz, 2<H), 2.52 (m, 4H), 2.20 (t, J=6 Hz, 2H), 1.89 (m, 4H), 86 (,2H), 1.53 (s, 9H);MS(ES) m/z 306 (M⁺+1).

### Example 106

### tert-Butyl 4-(3-pyrrolidin-1-ylprop-1-ynyl)pyridin-3-ylcarbamate

tert-Butyl 4-(3-hydroxyprop-1-ynyl)pyridin-3-ylcarbamate (1.1 g, 4.4 mmol) was dissolved under inert-gas atmosphere in methylene chloride (40 mL) and cooled to 0° C. Methanesulfonyl chloride (0.51 mL, 6.6 mmbl) and triethylamine,(1.9 mL, 13.2 mmol) were added and stirring was continued for 1.5 h. Pyrrolidine (1.9 mL, 22 mmol) was added and the reaction mixture was stirred for 12 h at room temnperature. Saturated aqueous sodium chloride solution (5 mL) was added and the organic layer was separated and dried over sodium sulfate. Filtration and removal of the solvent in vacuo yielded a residue, which was purified by chromatography on silica gel using a gradient ethyl acetate/heptane, (1:8), to ethyl acetate/methanol, (1:1), as the eluent to give 1.25 g (94% yield) of the title compound: ¹H NMR (CDCl₃, 400 MHz) S 9.36 (s, 1H), 8.22 (d, J=5 Hz, 1H), 7.21 (dd, J=5, 1 Hz, 1H), 7.07 (br s, 1H), 3.73 (s, 2H), 2.70 (m, 4H), 1.86 (m, 4H), 1.53 (s, 9H); MS (ES) m/z 302 (M⁺+1).

### Example 107

### tert-Butyl 5-(3-pyrrolidin-1-ylprop-1-ynyl)pyridin-3-ylcarbamate

The title compound was prepared as described for Example 106 using tert-butyl 5-(3-hydroxyprop-1-ynyl)pyridin-3-ylcarbamate, yield 82%: ¹H NMR (CDCl₃, 400 MHz) δ 8.34 (s, 1H), 8.31 (s, 1H), 6.71 (s, 1H), 2.88 (m, 4H), 1.92 (m, 4H), 1.51 (s, 9H); MS (ES) m/z 302 (M⁺+1).

### Example 108

### tert-butyl 4-[3-(dimethylamino)prop-1-ynyl]pyridin-3-ylcarbamate

tert-Butyl 4-iodopyridin-3-ylcarbamate (0.32 g, 1.0 mmol; described in: Crous, R. et al, Heterocycles, 1999, 51, 721-726), Pd(PPh₃)₄ (58 mg, 0.05 mmol), copper(1) iodide (19 mg, 0.1 Imnol), potassium carbonate (0.45 g, 3.0 mmol), 1-dimethylamino-2-propyne (0.323 ML, 3.0 mmol) were mixed with anhydrous tetrahydrofuran (3 mL) in a sealed reaction tube. All air was evacuated and tube was flushed with nitrogen for 5 min. The reaction mixture was heated to 55° C. over night. The mixture was filtered through Celite. Silica gel was added and the solvent was evaporated. Purification by chromatography on silica gel using a gradient, heptane to heptane/ethyl acetate, (2:1), as the eluent gave 188 mg (73% yield) of the title compound as oil: ¹H NMR (CDCl₃, 400 MHz) δ 9.49 (s, 1H), 8.38 (d, J=5 Hz), 7.37 (d, J=5 Hz, 1H), 3.77 (s, 2H), 2.55 (s, 6H), 1.67 (s, 9H); ¹³C NMR (CDCl₃, 100 MHz) δ 174.48, 151.88, 142.79, 140.17, 135.54, 125.00, 118.78, 81.54, 48.15, 43.64, 28.18, 21.25; MS (ES) ni/z 276 (M⁺+1).

### Example 109

### 4-(3-Dimethylaniinopropyl)pyridin-3-ylamine

tert-Butyl 4-[3-(dimethylamino)prop-1-ynyl]pyridin-3-ylcarbamate (0.31 g, 1.13 mmol) and palladium (1O %)on charcoal (10 mg) was mixed with methanol (25 mL). The reaction mixture was shaken under hydrogen atrnosphere (2 bar) for 3 h. The product mixture was filtered through Celite and the solvent was evaporated. The remaining oil was dissolved in trifluoroacetic acid (50% in methylen chloride, 10 mL) and stirred for 2 h. Evaporation of the solvent followed by purification by reversed phase chromatography (C-18), gradient water/acetonitrile and freeze-drying gave 0.202 g (99% yield) of the title compound: MS (ES) z 180 (M⁺+1).

### Example 110

### 5-(3-Pyrrolidin-1-ylpropyl)pyridin-3-amine

The title compound was prepared as described for Example 109 using tert-butyl 5-(3-pyrrolidin-1-ylprop-1-ynyl)pyridin-3-ylcarbamate. The solvent was evaporated and the crude residue was dissolved in methanol and basic ion exchange resin (Dowex OI) was added until the solution was basic. Filtering and evaporation of the solvent gave the title compound as a brown syrup, yield 99%: MS (ES) nVz 206 (MG+4).

### Example 111

### tert-Butyl 4-(3-hydroxyprop-1-ynyl)pyridin-3-ylcarbamate

tert-Butyl 4-iodopyridin-3-ylcarbamate (2.07 g, 6.5 mmol; described in: Crous, R. et al, Heterocycles, 1999, 51, 721-726), prop-2-yn-1-ol (0.45 mL, 7.7 mmol), copper(I) iodide (120 mg, 0.63 mmol), triethylamine (3 mL, 21.4 mmol) and Pd(PPh₃)₄ (80Mg, 0.07 mmol) were dissolved under inert gas atmosphere in tetrahydrofuran (40 mL). The reaction mixture was stirred for 12 h at 50° C. Water (10 mL) and saturated aqueous sodium chloride solution (40 mL) were added. The organic layer was separated and dried over magnesium sulfate. Filtration and removal of solvent in vacuo yielded a residue which was purified by chromatography on silica gel using a gradient heptanelethyl acetate, (1:10), to ethyl acetate/methanol, (1:1), as the eluent to give 1.3 g (81% yield) of the title compound as a solid: ¹H NMR (CDCl₃, 400 MH) δ 9.39 (s, 1H), 8.21 (d, J=5 Hz, 1H), 7.22 (d, J=5 Hz, 1H), 7.05 (s, 1H), 4.59 (s, 2H), 1.53 (s, 9H).

### Example 112

### tert-Butyl 5-(3-hydroxyprop-1-ynyl)pyridin-3-ylcarbamate

tert-Butyl 5-bromopyridin-3-ylcarbamate (4.0 g-14.3 mmol), propargylalcohol (1.6 g, 29 mmol), potassium carbonate (4.05 g, 29 mmol), copper(I)iodide (0.279 g, 1.423 mmol) and Pd(PPh₃)₄ (0.85 g 0.73 mmol) were mixed in tetrahydrofuran (25 mL) and heated to 65° C. over night. Evaporation of the solvent and absorption on silica gel followed by chromatography on a silica gel column using heptane to heptane/ethyl acetate, (1:1), gradient gave 1.0 g (28% yield) of the title compound, ¹H NMR (CD₃OD, 400 MHz) δ 8.41 (d, J=2 Hz, 1H) 8.08 (s, 1H), 7.91 (s, 1H), 4.32 (s, 2H), 1.42 (s, 9H)I; ³C (CD₃OD, 100 MHz) 8154.71, 145.81, 139.69, 137.95, 128,94, 121.67, 92.66,81.74, 81.61, 51.06, 28.55; MS (ES) m/z 249 (M⁺+1)

### Example 113

tert-Butyl 5-[3-(dimethylamino)prop-1-ynyl]pyridin-3-ylcarbamate The title compound was prepared as described for Example 112 using tert-butyl 5-bromopyridin-3-ylcarbamate and 1-dimethylamino-2-propyne, yield 91% as brown solid: ¹³C NMR (CDCl₃, 100 MHz) δ 152.52, 146.37, 138.87, 134.90, 127.98, 120.15, 87.15, 82.44,47.97,43.61, 28.23. MS (ES) m/z 276 (M⁺+1).

### Example 114

### tert-Butyl 5-bromopyridin-3-ylcarbamate

5-Bromonicotinic acid (10 g, 49.5 mmol), diphenylphosphorylazide (11.2 mL, 52 mmol) and triethylaminue (7.25 mL, 52 mmol) were mixed in'tert-butylalcohol (50 mL). The reaction mixture was stirred for 12 h at 60° C. and the solvent was evaporated in vacuo. The remaining crude product was diluted with methylene chloride (500 mL) and washed with HCl (aq) (100 mL, 0.2 M), water (100 mL), sat NaHCO₃ (aq) (100 mL) and water (100 IrSL). The organic phase was evaporated and purification by chromatography on a silica gel column using a gradient heptane to heptane/ethyl acetate, (2:1), gave 11 g (81% yield)of the title compound: ¹³C NMR (CDCl₃, 100 MHz) δ 152.97, 144.33, 137.67, 137.36, 128.78, 121.40, 82.18, 28.67; MS (ES) m/z 273 and 275 (M⁺+1).

### Example 115

### tert-Butyl 5-[3-(dimethylamino)propyl]pyridin-3-ylcarbamate

tert-Butyl 5-[3-(dimethylamino)prop-1-ynyl]pyridin-3-ylcarbamate (0.310 g, 1.126 mmol) and palladium (10%) on charcoal (10 mg) were mixed with methanol (25 mL) in a reaction bottle. Vacuum nitrogen cycle 3 times was performed to remove the air. The reaction mixture was shaken under hydrogen atmosphere (2 bar) for 1.5 h. The product mixture was filtered through celite and the solvent was evaporated. Chromatography on silica gel using methylene chloride to methylene chloride/ethanol, (2:1), as the eluent gave 1.8 g (89% yield) of the title compound: MS (ES) m/z 280 (M++I).

### Example 116

### 5-[3-(Dimethylamino)propyl]pyridin-3-amine

Trifluoroacetic acid, 50% in methylene chloride (10 mL), was added to a solution of tert-butyl 5-[3-(dimethylamino)propyl]pyridin-3-ylcarbamate (1.0 g, 3.58 mmol) and stirred for 2 h. Evaporation of the solvent followed by addition of methanol and treatment with DOWEX (8) OH gave after filtration and evaporation 0.60 g (94% yield) the title compound: ¹H NMR (CD₃OD 400 MHz) δ 7.63 (m, 2H), 7.71 (m, 1H), 7.32 (m, 1H), 3.04(m11,2H) 2.91 (m, 2H), 2.63 (m, 6H), 2.50 (t, J=8 Hz, 2H); ³CNMR (CD₃OD, 100 MHz) δ 149.88, 142.37, 129.41, 128.74, 125.12, 57.93, 43.43, 30.14, 26.21; MS (ES) m/z 180 (M⁺+1).

### Example 117

### 2-Amino-5-bromo-N-(3-pyridinyl)benzamide

Triethylaluminium (8.7 mL, 17.4 mmol) was added dropwise to a solution of methyl-2-amino-5-bromobenzoate (2 g, 8.69 mmol) and 3-arinopyridine (0.82 g, 8.69 mmol) in methylene chloride (20 mL) at roorn temperature (N₂-atm). The mixture-was refluxed for 5 days and ice and water was added in portions. The organic solution was washed, twice, with water, dried (MgSO₄) and evaporated in vacuo to give 0.143 g (6% yield) of the title compound as a yellow solid: ¹H NMR (DMSO-d₆, 400 MHz) δ 10.26 (s, 1H), 8.85 (d, J=2 Hz, 1H), 8.29 (dd, J=4, 1 Hz, 1H), 8.09 (m, 1H), 7.81 (d, J=2 Hz, 1H), 7.35 (m, 2H), 6.74 (d, J=9 Hz, 1H), 6.54 (br s, 2H); ¹³C NMR (DMSO-d₆, 100 MHz) δ 166.8, 20-149.2, 144.5, 135.6, 134.9, 130.7, 127.6, 123.4, 118.6, 115.8, 105.0; MS (ES) m/z 29² and 294 (M⁺+1).

### Example 118

### 2-Amino-5-bromo-N-[4-(pyrrolidin-1-ylmethyl)pyridin-3-yl]nicotinamide

2-Amino-5-broinonicotinic acid (60 mg, 0.28 mmol), 4-(pyrrolidin-1-ylmethyl)pyridin-3-amine (60 mg, 0.34 mmol), 2-(1H-benzotriazol 1-yl)-1,1,3,3-tetramethyluroniumtetrafluoroborate (133 mg, 0.41 mmol), 1-hydroxybenzotriazole hydrate (56 mg, 0.41 mmol) and N,N-diisopropylethylamine (0.1 mL, 0.6 mmol) were suspended in acetonitrile (8 mL) and stirred at room temperature for 12 h. The solvent was removed in vacuo and the residue was separated between methylene chloride arid saturated aqueous sodium hydrogen carbonate solution and the organic layer was dried over sodium sulfate. Filtration and removal of solvent in vacuo gave the crude product which was purified by chromatography on silica gel using a gradient ethyl acetate/heptane, (1:1) to (10:1), as the eluent to give 96 mg (93% yield) of the title compound as a solid: ¹H NMR (DMSO-d₆, 400 MHz) δ 11.69 (s, 1H), 9.26 (s, 1H), 8.28 (d, J5=Hz, 1H), 8.24 (d, J=3 Hz, 1H), 7.95 (d,J 3 Hz, 1H), 7.34 (brs, 2H), 7.32 (d, J=4 Hz, 1H), 3.83 (s, 2H), 2.53 (m, 4H), 1.79 (m, 4H); MS (ES) m/z 376 and 378 (M⁺+1).

### Example 119

### 3-Amino-6-bromo-N-[4-(pyrrolidin-1-ylmethyl)pyridin-3-ylpyrazine-2-carboxamide

3-Amino-6-6-bromopyrazine-2-carboxylic acid (148 mg, 0.68 mmol; described in: Ellingson, R. C.; Henry, R. L. J. Am. Chem. Soc. 1949, 2798-2800), 4-(pyrrolidin-1-ylmethyl)pyridin-3-amine (100 mg, 0.56 mmol), 2-(1H-benzotriazol-1-yl)-1,1,3,3-tetramethyluroniumtetrafluoroborate (288 mg, 0.89 mmol), 1-hydroxybenzotriazole hydrate (118 mg, 0.87 mmol) and N,N-diisopropylethylamine (0.2 mL, 1.15 mmol) were suspended in acetonitrile (8 mL) and stirred under inert gas atmosphere at room temperature for 12 h. The solvent was removed in vacuo and the residue was separated between methylene chloride and saturated aqueous sodium hydrogen carbonate solution and the organic layer was dried over sodium sulfate. Filtration and removal of solvent in vacuo gave the crude product, which was purified by chromatography on silica gel using a gradient ethyl acetate/heptane, (1:1) to (4:1), as an eluent to give 210 mg (98% yield).of the title compound as a light brown solid: ¹H NMR (DMSO-d₆,400 MHz) δ 11.97 (s, 1H), 9.41 (s, 11H), 8.46 (s, 1H), 8.30 (d, J=5 Hz, 1 H), 7.84 (br s, 2H), 7.34 (d, J=5 Hz, 1H), 3.77 (s, 2H), 2.57 (m, 4H), 1.84 (m, 4H).

The following Examples, 120-121, were synthesized as described for Example 119:

### Example 120

### 3-Amino-6-bromo-N-[4-(2-pyrrolidin-1-ylethyl)pyridin-3-yl]pyrazine-2-carboxamide

Starting material: 4-(2-pyrrolidin-1-ylethyl)pyridin-3-amine. Purification by chromatography on silica gel using a gradient ethyl acetatelmethanol, (10:1), to ethyl acetatelmethanol/triethyl amine, (4:1:0.05), as the eluent gave the title compound as a brown oil, yield 91%: ¹H NMR (DMSO-d₆, 400 MHz) 610.51 (br s, 1H), 8.68 (s, 1H), 8.43, s (1H), 8.33 (d, J=5 Hz, 1H), 7.72 (br s, 2H), 7.35 (d, J=5 Hz, 1H), 2.77 (m, 2H), 2.67 (m, 2H), 2.49 (m, 4H), 1.63 (m, 4H).

### Example 121-

### 3-Amino-6-bromo-N-{4-[(dimethylamino)methyl]pyridin-3-yl}pyrazine-2, carboxamide

Starting material: 4-[(dimethylamino)methyl]pyridin-3-amine. Purification by chromatography on silica using a gradient ethyl acetate/heptane, (4:1), to ethyl acetate/methanol, (2:1), as an eluent gave the title compound as a yellow solid, yield 70%: ¹H NMR (CD₃OD, 400 MHz) δ 9.53 (s, 1H), 8.26 (s, 1H), 8.20 (d, J=5 Hz, 1H), 7.26 (d, J=5 Hz, 1H), 3.62 (s, 2H), 2.36 (s, 6H); MS (ES) m/z 351 and 353 (M⁺+1).

### Example 122

### 3-Amino-6-bromo-N-{S-[3-(dimethylamino)propyl]pyridin-3-yl}pyrazine-2-carboxamide

1-Ethyl-3-(3-dimethylaminopropyl)carbodiimide hydrochloride (0.81 g, 4.2 mmol) and 1-hydroxybenzotriazole (0.57 g, 4.2 mmol) were added to a mixture of 5-(3-dimethylamino-propyl)pyrdin-3-ylamine (0.345 g, 1.93 mmol), 3-amino-6-bromopyrazine-2-carboxylic acid (0.546 g, 2.5 mmol, described in Ellingson, R. C., Henry, R. L., J. Am. Chem. Soc. 1949, 71, 2798-2800) in N,N-dimethylformamide (2 mL) at 0° C. The mixture was stirred for 30 min. Precipitation was almost immediate, filtering the precipitate and washing with diisopropyl ether gave 0.402 g (55% yield) of the title compound: ¹H NMR (CDCl₃/CD₃OD (1:1), 400 MHz) δ 8.75 (s, 1H), 8.23 (s, 1H), 8.13 (s, 1H), 8.11 (s, 1H), 3.12 (m, 4H), 2.81 (s, 6H), 2.70 (dd, J=8, 8 Hz, 2H), 2.01 (m, 4H); MS (ES) m/z ³⁷⁹ and 381(M+1).

### Example 123

### 3-Amino-6-bromo-N-[5-(3-pyrrolidin-1-yipropyl)pyridin-3-yl]pyraine-2 carboxamide

1-Ethyl-3-(3-dimethylaminopropyl)carbodiimide hydrochloride (0.81 g, 4.2 mmol) and 1-hydroxybenzotriazole (0.57 g, 4.2 mmol) were added to a mixture of 5-(3-pyrrolidin-1-yl-propyl)pyridin-3-ylamine (0.3 g, 1.46 mmol), 3-amino-6-bromropyrazine-2-carboxylic acid (0.382 g, 1.76 mmol; described in: Ellingson, R. C.; Henry, R. L. J. Am. Chen. Soc. 1949, 2798-2800) in N,N-dimethylformamide (2 mL) at 0 IC. The mixture was stirred for 1 h. The solvent was evaporated and the crude product was purified by chromatography on a silica gel column using a gradient methylene chloride to methylene chloride/methanol, (2:1), to give 0.456 g, (77% yield) of the title compound: ¹H NMR (CDCl₃/CD₃OD (1:1), 400 MHz)68.85 (d, J=2 Hz, 1H), 8.36 (s, 1H), 8.24 (d, J=2 Hz, 1H), 8.20 (dd, J=2, 2 Hz, 1H), 2.91 (m, 4H), 2.84 (m, 2H), 2.80 (m, 2H), 2.04 (m, 2H), 1.98 (m, 4H); ¹³C NMR (CDCl₃/CD₃OD (1:1),400 MHz) δ 163.52, 153.98, 149.13, 143.65, 138.60, 136.79, 134.34, 127.35, 124.09, 121.63, 54.49, 53.27, 29.47, 28.01, 22.29; MS (ES) niz 405 and 407 (M⁺+1).

### Example 124

### Methyl 3-amino-6-{4-[(dimethylamino)sulfonylphenyl}pyrazine-2-carboxylate

3-Amino-6-bromopyrazine-2-carboxylic acid methyl ester (0.40 g, 1.72 mmol), 4-(—N₁N-1S dimethylsulfonamide)phenylboronic acid (0.474 g, 2.07 mmol) and Pd(dppf)Cl₂ (63 mg, 86.2 μmol) were mixed in toluene/ethanol, (1: 1, 2 mL), and Na₂CO₃ (2 M (aq), 0.40 mL). Nitrogen gas was bubbled through the reaction mixture for 5 min and the mixture was heated to 80° C. for 16 h. Silica gel was added and the solvent was evaporated. The residue was purified by chromatography on a silica gel column using a gradient, heptane to heptane/ethyl acetate, (2:1), as the eluent to give 0.40 g (69% yield) as a yellow solid: ¹H NMR (CD₃OD, 400 MHz) 8.64 (s, 1H), 7.86 (d, J=9 Hz, 2H), 7.58 (d, J=9 Hz, 2H), 3.73 (s, 3H), 2.45 (s, 6H); MS (ES) m/z337 (M⁺+1).

### Example 125

### 3-Amino-6-{4-[(dimethylamino)sulfonyl]phenyl}pyrazine-2-carboxylic acid

Methyl 3-amino-6-{4-[(dimethylamino)sulfonyl]phenyl}pyrazine-2-carboxylate (0.25 g, 0.74 mmol) and lithium hydroxide (0.20 g, 8.35 mmol) were mixed in tetrahydrofuran/water, (10:1, 50 mL), and stirred for 2 h. The solvent was evaporated and the residue was dissolved in water and washed with chloroform. The phases were separated and the water-phase was acidified with.HCl (aq) (2 M). Extraction with chloroform/diethyl ether, (20:1), gave after evaporation 0.21 g, (87% yield) the title compound as a yellow solid: MS (ES) m/z 323 (M⁺+1).

### Example 126

### tert-Butyl 4-formylpyridin-3-ylcarbamate

tert-Butllitium (13.3 mL, 22.7 mmol) was added dropwise to a cooled (−78° C.) solution of 3-(tert-butoxycarbonylamino)pyridine (2.0 g, 10.3 mmol; described in: Kelly, T. A., McNell, D. W. Tetrahedron Lett. 1994, 35, 9003-9006) in anhydrous tetrahydrofuran (20 mL) under nitrogen atmosphere. The reaction mixture was stirred at −78° C. for 3 h. N-Formylpiperidine-(1.4: mL, 12.4 mmol) was added dropwise to the cooled reaction mixture and stirring was continued for 1 h. Water (5 mL) was added and the mixture was stirred for min. The crude reaction-mixture was pre adsorbed onto silica and purified by chromatography on silica gel using a gradient heptane to heptane/ethyl acetate, (2:1), to give 1.83 g (80% yield) of the title compound as a yellowish solid: ¹H NMR (CDCl₃, 400 MHz) δ 9.92 (s, 1H), 9.81 (s, 1H), 9.74 (s, 1H), 8.44 (d, J=5 Hz, 1H), 7.45 (d, J.=6 Hz, 1H), 1.48 (s, 9H); ¹³C NMR (CDCl₃, 100 MHz) 142.87, 141.87, 135.29, 125.93, 124.45, 81.64, 28.03; MS (ES) m/z 195 (M⁺+1).

### Example 127

### 3-Amino-6-[4-(pyrrolidin-1-ylsulfonyl)phehyl]pyrazine-2-carboxyic acid.

Methyl 3-amino-6-[4-(pyrrolidin-1-ylsulfonyl)phenyl]pyrazine-2-carboxylate (1.0 g, 2.76 mmol) and lithium hydroxide (1.0 g 24 mmol) were mixed in tetrahydrofuran/water, (9:1, mL) and stirred at room temperature over night for 18 h. The reaction mixture was evaporated and the crude product was purified by reversed phase chromatography (C-18) using water/acetonitrile gradient to give 0.85 g (88% yield) of the title compound in: MS (ES) m/z 349 (M⁺+1).

### Example 128

### Methyl 3-amino-6-[4-(pyrrolidin-1-ylsulfonyl)phenyl]pyrazine-Z-carboxylate

4-(Pyrrolidylsulfonamide)phenylboronic acid (0.33 g, 1.29 mmol), methyl 3-amino-6-bromopyrazine-2-carboxylate (0.25 g, 1.08 mmol), K₃PO₃ (1.1 mL, 3 M, 3.2 mmol), and Pd(dppf)Cl₂ (0.044 g, 54 mmol) were suspended in ethylene glycol dimethyl ether/water (1.5:0.5 mL) and heated in a microwave oven at 160° C. for 10 min. The reaction was repeated 3×. The combined product mixtures were evaporated with silica gel and the crude product was purified by chromatography on silica gel using a heptan/ethylacetate gradient to give 0.96 g (82% yield) of the title compound: MS (ES) m/z 363 (M⁺+1).

End Compounds

### Example 129

### 3-Amino-N-pyridin-3-yl-6-[4-(pyrrolidin-1-ylsulfonyl)phenyl]pyrazine-2-carboxamide

3-Amino-6-bromo-N-pyridin-3-ylpyrazine-2-carboxamide (0.25 g, 0.85 mmol), 4-(pyrrolidin-1-ylsulfonyl)phenylboronic acid (0.26 g, 1.02 mmol), Pd(dppf)Cl₂ (35 mg, 42-0o pmol) and sodium carbonate (2 M, 1.5 mL, 3.0 mmol) were mixed with dimethoxyethane in a schlenk tube and nitrogen gas was flushed through the reaction tube mixture for 5 min. The mixture was heated to reflux for 1 h. Silica gel was added and the solvent was evaporated. The residue was purified by chromatography on a silica gel column, using a gradient heptane to heptane/ethyl acetate, (2:1), as the eluent to give 0.335 g (93% yield) as a yellow solid: MS (ES) m/z 425 (M⁺+1).

### Example 130

### 3-Amino-6-[4-(piperidin-1-ylsulfonyl)phenyl]-N-pyridin-3-ylpyrazine-2-carboxamide

The title compound was prepared as described for Example 129 using 4-(piperidin-1-ylsulfonyl)phenylboronic acid, yield 99%: MS (ES) m/z 439 (M⁺+1).

The following Examples, 131-133, were synthesized as described for Example 237

### Example 131

### 3-Amino-6-{3-ethyl-4-[(4-methylpiperazin-1-yl)sulfonylphenyl]-N-pyridin-3-ylpyrazine-2-carboxamide

Starting material: 3-ethyl-4-[(4-methylpiperazin-1-yl)sulfonyl]phenylboronic acid. The crude product was purified by column chromatography on silica using methylene chloride/methanol, (95:5), as the eluent, yield 62%: ¹H NM (CDCl₃) δ 9.88 (s, 1H), 8.83 (s, 1H), 8.74 (s, 1H), 8.45 (s, 1H), 8.30 (m, 1H), 8.01 (m, 1H), 7.88 (s, 1H), 7.82 (m, 1H), 7.38 (m, 1H), 3.33 (br s, 4H), 3.12 (m, 2H), 2.62 (br s, 4H), 2.39 (s, 3H), 1.38 (m, 3H); ¹³CNMR (CDCl₃) δ 164.4, 154.8, 145.9, 145.8, 145.4, 141.7, 140.5, 138.8, 135.0, 134.3, 131.4, 128.3, 127.1, 124.5, 124.0, 123.3, 54.3, 45.7, 45.0, 26.6, 16.1; MS (TSP) m/z 482 (M⁺+1).

### Example 132

### 3-Amino-6-[4-[(4-methylpiperazin-1-yl)sulfonyl]-3-(trinuoromethoxy)phenyl]-N-pyridin-3-ylpyrazine-2-carboxamide

Starting material: 4-[(4-methylpiperazin-1-yl)sulfonyl]-3-(trifluoromethoxy)phenylboronic acid. The-crude product was purified by column chromatography on silica using methylen chloride/methanol, (95:5), yield 70%: ¹H NMR (CDCl₃) δ 9.79 (s, 1H), 8.83 (s, 1H), 8.75 (s, 1H), 8.45 (m, 1H), 8.27 (d, J=8 Hz, 1H), 8.09 (d, J=8 Hz, 1H), 7.97 (s, 1H), 7.89 (d, J=8 Hz, 1H), 7.38 (dd, J=8,5-Hz, 1H), 3.41 (br s, 4H), 2.64 (br s, 4H), 2.41 (s, 3H); MS (TSP) m/z 538 (M⁺+1).

### Example 133

tert-Butyl 4-[(4-{5-amino-6-[(pyridin-3-ylamino)carbonyl]pyrazin-2-yl}phenyl)sulfonyl]piperazine-1-carboxylate

Starting material: 4-([4-(tert-butoxycarbonyl)piperazin-1-yl]sulfonyl}phenylboronic acid. The crude product was purified by column chromatography on silica using methylen chloride/methanol, (95:5), yield 60%: ¹H NMR (CDCl₃) δ 9.85 (s, 1H), 8.86 (s, 1H); 8.76 (s, 1H), 8.45 (d, J=S Hz, 1H), 8.30 (d, J=8Hz, 1H), 8.07 (d, J=8 Hz, 2H), 7.89 (d, J=8 Hz, 2H), 7.38 (dd, J=5, 8 Hz, 1H), 3.54 (br s, 4H), 3.04 (br s, 4H), 1.40 (s, 9H); ¹³CNMR (CDCl₃) δ 164.4, 154.9, 154.3, 145.9, 145.8, 141.7, 140.5, 138.5, 135.5, 134.2, 128.8, 127.4, 126.4, 124.6, 124.0, 80.7, 46.1, 28.5; MS (TSP) m/z 540 (M⁺+1)

### Example 134

### 3-Amino-N-{5-[3(dimethylamino)propyl]pyridin-3-yl}-6-[4-(piperidin-ylsulfonyl)phenyl]pyrazine-2-carboxamide

4-piperidin-1-ylsulfonyl)phenylboronic acid (0.49 g, 0.55 mmol), 3-amino-6-bromo-N-{5-[3-(dimethylamino)propyl pyridin-3-yl}pyrazine-2-carboxamide (0.175 g, 0.46 mmol), Na₂CO₃ (0.147 g, 1.38 mmol), and Pd(dppf)Cl₂ (0.019 g, 23 lmol) were suspended in ethylene glycol dimethyl ether/water, (3:1 mL) and heated in a microwave oven at 160° C. for 10 min. The product mixture-was filtered through celite, diluted with methylene chloride (25 mL), washed with sodium hydroxide (aq, 1 M) and water. The organic phase was dried (Na₂SO₄) and evaporated to give 0.197 g, (82% yield) of the title compound: MS (ES) m/z 524 (M⁺+1).

### Example 135

### 3-Amino-N-{5-[3-(dimethylamino)propyl]pyridin-3-yl}-6-[4-(pyrrolidin-1-ylsulfonyl)phenyl]pyrazine-2-carboxamide

The title-compound was prepared as described for Example 134 using 4-(pyrr lidin-1-ylsulfonyl)phenylboronic acid, yield 73%: MS (ES) nz/z 510 (M+1).

### Example 136

### 3-Amino-N-{4-[(dimethylamino)methyl]pyridin-3-yl}-6-{4[(dimethylamino)sulfonyl]phenyl}pyrazine-2-carboxamide

3-Amino-6-{4-[(dimethylamino)sulfonyl]phenyl}pyrazine-2-carboxylic acid (71 mg, 0.22 mmol), 4[(dimethylamino)methyl]pyridin-3-amine (40 mg, 0.265 mmol) and bromo-tripyrrolidinophosphoniumhexafluorophosphat (0.154 g, 0.33 mmol) were mixed in N,N-dimethylformamide (2 mL) and stirred for 5 min. N₁N-Diisopropylethylamine (90-1 ml, 0.66 mmol) was added and the reaction mixture was stiired for 15 h, The solvent was evaporated and the crude residue was dissolved in HCl (1 M aq, 2 mL) and applied on a reversed phase chromatography column (XTerra C8 19×300 mm) and eluted with a water/acetonitrile gradient. Freeze-drying gave 42 mg (42% yield) of the title compound as a yellow solid: MS (ES) ?ni/z 456 (M⁺+1).

### Example 137

### 3-Amino-N-{4-[3-(dimethylamino)propyl]pyridin-3-yl}-6-{0.4[(dimethylaminxo)sulfonyl]phenyl}pyrazine-2-carboxamide

The title compound was prepared as described for Example 136 using 3-amino-6(4-[(dimethylamino)sulfonyl]phenyl)pyrazine-2-carboxylic acid and 4-(3-dimethylaminopropyl)pyridin-3-amine. The title compound was purified on a.reversed phase column (Xerra C8 19×300 mm) and eluted with a water/acetonitrile gradient to give 25 mg (7% yield) as a yellow solid: ¹H NMR (CD₃OD, 400 MHz) δ 8.79 (s, 1H), 8.78 (d, J=10 Hz), 8.25 (m, 1H), 8.23 (d, J=9 Hz, 2H), 7.77 (d, J=9 Hz, 2H) 7.33 (d, J=5 Hz, 1H), 2.73 (t, J=8, 8 Hz, 2H), 2.60 (s, 6H), 2.54 (t, J=8 Hz), 2.28 (s, 6H), 1.85 (m, 2H); MS (ES) m/z 484 (M⁺+1).

### Example 138

### 3-Amino-6-(4-{[methyl(1-methylpyrrolidin-3-yl)amino]sulfonyl}phenyl-N-pyridin-3-ylpyrazine-2-carboxamide

A mixture of 4-((methyl(1-methylpyrrolidin-3-yl)amino)sulfonyl)phenylboronic acid (298 mg, 1 mmol), 3-amino-6-bromo-N-pyridin-3-ylpyrazine-2-carboxamide (294 mg; 1 mmol) and Pd(dppf)Cl₂×CH₂Cl₂ (42 mg, 0.05 mmol) in toluene (10 mL), ethanol (2 mL) and saturated aqueous sodium carbonate solution (2 mL) was stirred at 80° C. for 16 h. The mixture was cooled to room temperature, and precipitated material was filtered off, dissolved in aqueous HCl (1 M, 5 mL), alkalyzed with aqueous NaOH (2 M) and extracted with methylene chloride. The organic phase is washed with water, dried (Na₂SO₄), evaporated to dryness and chromatographed on silica using methylene chloride/mnethanol, (10:1), as the eluent to give 0.103 mg (22% yield) of the title compound: MS (ES) 448 (M⁺+1).

The following Examples 139-152 were synthesized as described for Example 138:

### Example 139

### 3-Amino-6-(4-{[methyl(1-methylpiperidin-4-yl)amino]sulfonyl}phenyl)-N-pyridin-3-ylpyrazine-2-carboxamide

Starting material: 4-((methyl-(1-methylpiperidin-4-yl)amino)sulfonyl)phenylboronic acid, yield 4%: MS (ES) 482 (M⁺+1).

### Example 140

### 3-Amino-6-(4-{[[3-(dimethylamino)propyl](methyl)aminoisulfonyl}phenyl)-N-pyridin-3-ylpyrazine-2-carboxamide hydrochloride

Starting material: 4-(((dimethylamino)propyl)(methyl)amino)sulfonyl)phenylboronic acid, yield 20%.The compound was dissolved in 1 M HCl(q, evaporated and freeze dried: MS (ES) 470 (M++I).

### Example 141

### 3-Amino-6-(4-{[3-(dimethylamino)pyrrolidin-1-yl]sulfonyl}phenyl)-N-pyridin-3-ylpyrazine-2-carboxamide

Starting material: 4-((3-dimethylamino)pyrrolidin-1-yl)sulfonyl)phenylboronic acid, yield 22%: MS (ES) 468 (M⁺+1).

### Example 142

### 3-Amino-6-[4-(morpholin-4-ylsulfonyl)phenyl]-N-pyridin-3-yipyrazine-2-carboxamide

Starting material: 4-(morpholin4-ylsulfonyl)phenylboronic acid. Purification on a silica gel column using methylene chloride/methanol, (100:1), as the eluent gave the title compound, yield 18%: MS (ES) 441 (M⁺+1).

### Example 143

### 3-Amino-6-[4-({[3-(4-methylpiperazin-1-yl)propyl]amino}sulfonyl)phenyl]-N-pyridin-3-ylpyrazine-2-carboxamide hydrochloride

Starting material: 4-(((3-(4-methylpiperazin-1-yl)propyl)amino)sulfonyl)phenylboronic acid, yield 27%. The compound was dissolved in 1 M HCl, evaporated and freeze dried: MS (ES) 511 (M++I).

### Example 144

### 3-Amino-6-{4-[(4-ethylpiperazin-1-yl)sulfonyl]phenyl}-N-pyridin-3-ylpyrazine-2-carboxamide

Starting material: 4((4-ethylpiperazin-1-yl)sulfonyl)phenylboronic acid, yield 62%: MS (ES) 468 (M⁺+1).

### Example 145

### 3-Amino-N-pyridin-3-yl-6-(4-{[(2-pyrrolidin-1-ylethyl)amino]sulfonyl}phenyl)pyrazine-2-carboxamide hydrochloride

Starting material: 4((2-pyrrolidin-1-ylethyl)amino)sulfonyl)phenylboronic acid, yield 30%. The compound was dissolved in 1 M HCl(.q, evaporated and freeze dried: MS (ES) 468 (M⁺+1).

### Example 146

### 3-Amino-6-{4-[(4-methyl-1,4-diazepan-1-yl)sulfonyl]phenyl}-N-pyridin-3-ylpyrazine-2-carboxamide hydrochloride

Starting material: 4-((4-methyl-1,4-diazepan-1-yl)sulfonyl)phenylboronic acid, yield 5%. The compound was dissolved in 1 M HCl(q), evaporated and freeze dried: MS (ES) 468(M⁺+1).

### Example 147

### 3-Amino-6-[4-({[2-(dimethylamino)propyl]amino}sulfonyl)phenyl]-N-pyridin-3-ylpyrazine-2-carboxamide hydrochloride

Starting material: 4-(((2-dimethylamino)jpropyl)amino)sulfonyl)phenylboronic acid, yield 26%: MS (ES) 456 (M⁺+1). The base was dissolved in methylene chloride/methanol, (9/1), and HCl in diethyl ether (2 M) was added to acidic pH. The formed precipetate was filtered and dried in vacuo to give the title compound, yield 90% (from the base). ¹H NMR (DMSO-d₆) δ 11.09, (s, 1H), 9.36 (d, J=2 Hz, 1H), 9.09 (s, 1H), 8.84 (d, J=9 Hz, 1H), 8.66 (m, 1H), 8.51 (d, J=9 Hz, 2H), 8.28 (t, J=6 Hz, 1H), 7.99 (dd, J=9, 6 Hz, 1H), 7.94 (d, J=9 Hz, 2H), 3.37 (m, 1H), 3.19 (m, 1H), 2.97 (m, 1H), 2.72 (d, J=Hz, 3 H), 2.66 (d, J=5 Hz, 3H), 1.22 (d, J=5 Hz, 3H).

### Example 148

### 3-Amino-6-(4-{[isopropyl(2-methoxyethyl)amino]sulfonyl}phenyl)-N-pyridin-3-ylpyrazine-2-carboxamide hydrochloride

Starting material 4-((isopropyl-(2-methoxyethyl)amino)sulfonyl)phenylboronic acid, yield 43%. The compound was dissolved in 1 M HCl (aq), evaporated and freeze dried; MS (ES) 471 (M⁺+1).

### Example 149

### 3-Amino-6-[4-({[(-ethylpyrrolidin-2-yl)methyl]amino]sulfonyl)phenyl}-N-pyridin-3-ylpyrazine-2-carboxamide hydrochloride

Starting material: 4-((((1-ethylpyrrolidin-2-yl)amino)-sulfonyl)phenylboronic acid, yield 8%. The compound was dissolved in 1 M HCl_{(aq)} evaporated and freeze dried: MS (ES) 482 (M⁺+1).

### Example 150

### 3-Amino-6-[4-(}[2-(diethylamino)ethyl]amino}sulfonyl)phenyl]-N-pyridin-3-ylpyrazine-2-carboxamide hydrochloride

Starting material: 4-(((2-diethylamino)ethyl)amino)sulfonyl)phenylboronic acid, yield 81%. The compound was dissolved in 1 M HCl(,q), evaporated and freeze dried: ¹H NMR (D₂O, 400 MHz) δ 8.90 (s, 1H), 8.25 (m, 2H), 8.08 (s, 1H), 7.66 (m, 1H), 7.56 (d, 2H), 7.31 (d, 2H), 3.07 (m, 9H), 1.11 (t, 6H); MS (ES) 470 (M⁺+1).

### Example 151

### 3-Amino-N-pyridin-3-yl-6-(4-{[(2-pyridin-2-ylethyl)amino]sulfonyl}phenyl)pyrazine-2-carboxamide

Starting material: 0.4-(((2-pyridin-2-ylethyl) amino)sulfonyl)phenylboronic acid. Purification on a silica gel column using methylene chloride/methanol, (50:1), as the eluent gave the title compound, yield 12%: MS (ES) 476 (M⁺+1).

### Example 152

### 3-Amino-6-(4-{[(2-methoxy-1-methylethyl)amino]sulfonyl}phenyl)-N-pyridin-3-ylpyrazine-2-carboxamide hydrochloride

Starting material: 4-(((2-methoxy-1-methylethyl)amino)sulfonyl)phenylboronic acid. Purification on a silica gel column using methylene chloride/methanol, (50:1), as the eluent gave the title compound, yield 80%. The compound was dissolved in 1 M HCl_{(aq)}, evaporated and freeze dried: ¹H NMR (D₂O, 400 MHz) □ 9.40 (s, 1H), 8.73 (d, 1H), 8.66(s, 1H), 8.45 (d, 1H), 8.12 (d, 2H), 7.95 (dd, 1H), 7.75 (d, 2H), 3.25-3.30 (m, 1H), 3.04-3.14 (m, 2H), 3.06 (s, 3H), 0.84 (d, 3H); MS (ES) 443 (M⁺+1).

### Example 153

### 3-Amino-6-[4-({[2-(dimethylamino)-1-methylethyl]amino}sulfonyl)phenyl]-N-pyridin. ylpyrazine-2-carboxamide

To a mixture of 4-(((2-dimethylamino)-1-methylethyl)amino)sulfonyl)phenylboronic acid (286 mg, 1 mmol), 3-amino-6-bromo-N-3-ylpyrazine-2-carboxamide (235 mg, 0.8 mmol) and Pd(dppf)Cl₂×CH₂Cl₂ (42 mg, 0.05 mmol) was added tetrahydrofuran (3 mL) and a saturated aqueous sodium carbonate solution (1 mL) in a microwave vial. The mixture was subjected to microwave irradiation for 15 min at 160° C. The mixture was cooled to room temperature, and precipitated material was filtered off, dissolved in 1 M aqueous HCl (5mL), alkalyzed with aqueous NaOH and extracted with methylene chloride. The organic phase was washed with water, dried (Na₂SO₄), evaporated to dryness and chromatographed on silica using methylene chloride/methanol, (10:1), as the eluent to give 67 mg (15% yield) the title compound: MS (ES) 456 (M⁺+1).

The following Examples 154-157 were synthesized as described for Example 153:

### Example 154

### 3-Amino-N-pyridin-3-yl-6-(4-{[(3-pyrrolidin-1ylpropyl)amino]sulfonyl}phenyl)pyrazine-2-carboxamide

Starting material: 4-((3-pyrrolidin-1-ylpropyl)amino)sulfonyl)phenylboronic acid, yield 5%: ¹H NMR (CDCl₃, 400 M 8z) δ9.85 (s, 1H), 8.82 (s, 1H), 8.73 (s, 1H), 8.41 (d, 1H), 8.26 (m, 1H), 8.01 (d, 2H), 7.95 (d, 2H), 7.34 (dd, 1H), 3.11 (t, 2H), 2.62 (t, 2H), 2.57 (m, 4H), 1.82 (m, 4H), 1.71 (t, 2H); MS (ES) 482 (M⁺+1).

### Example 155

### 6-{4-[(4-Acetylpiperazin-1-yl)sulfonyl]phenyl}-3-amino-N-pyridin-3-ylpyrazine-2-carboxamide.

Starting material: 4-((4-acetylpiperazin-1-yl)sulfony)phenylboronic acid, yield 2%:MS (ES) 482 (M⁺+1).

### Example 156

### 3-Amino-6-(4-([[2-(dimethylamino)ethyl](ethyl)amino]sulfonylphenyl)-N-pyridin-3-ylpyrazine-2-carboxamide hydrochloride.

Starting material: 4-(((2-dimethylamino)ethyl)(ethyl)amino)sulfonyl)-phenylboronic acid.

The compound was precipitated as the hydrochloride salt, yield 26%: ¹H NMR. (D₂O, 400 MHz) δ8.92 (s, 0.1H), 8.27 (m, 2H), 8.07 (s, 1H), 7.69 (m, 1H), 7.57 (d, 2H), 7.32 (d, 2H), 3.28,(m, 2H), 3.18 (m, 2H), 2.97 (m, 2H), 2.77 (s, 6H), 0.75 (t, 3H); MS (ES) 470 (M⁺1+).

### Example 157

### 3-Amino-6-[4-({[3-(dimethylamino)propyl]amino}sulfonyl)phenyl]-N-pyridin-3-ylpyrazine-2-carboxamide hydrochloride

Starting material: 4-(((3-dimethylamino)propyl)amino)sulfonyl)phenylboronic acid, yield 22%: ¹H NMR (D₂O, 400 MHz) δ 8.95 (s, 1H), 8.29 (m, 2H), 8.12 (s, 1H), 7.71 (m, 1H), 7.58 (d, 2H), 7.32 (d, 2H), 3.04 (t, 2H), 2.80 (t, 2H), 2.73 (s, 6H), 1.76 (m, 2H). The compound was dissolved in 1 M HCl(aq), evaporated and freeze dried: MS (ES) 456 (M⁺+1).

### Example 158

### 2-Amino-S-{4-[(4-methylpiperazin-1-yl sulfonyl]phenyl}-N-[4-(pyrrolidin-1-ylmethyl)pyridin-3-yl]nicotinamide

4-[(4-Methylpiperazin-1-yl)sulfonyl]phenylboronic acid (117 mg, 0.41 mmol), 2-amino-5-bromo-N-[4-(pyrrolidin-1-ylmethyl)pyridin-3-yl]nicotinamide (54 mg, 0.14 mmol), sodium carbonate (50 mg, 0.47 mmol), Pd(dppf)Cl₂×CH₂Cl₂ (28 mg, 0.04 mmol) were suspended in ethylene glycol dimethyl ether/water (2.5:0.6 mL) and heated in a microwave oven at −160° C. for 10 min. Silica was added and the solvent was evaporated. Purification by column chromatography using ethyl acetate to ethyl acetate/methanol, (10:1), as the eluent gave a product which was further purified by reversed phase chromatography (column: XTerra C8 19×300 mm, gradient: water/acetonitrile/ammonium acetate). After removal of the solvent, the residue was dissolved in methylene chloride and the organic layer was washed with aqueous saturated sodium hydrogen carbonate solution and subsequently dried over sodium sulfate. Filtration and removal of solvent in vacuo gave 65 mg (8-7% yield) of the title compound as a yellow oil: ¹H NMR (CDCl₃, 400 MHz) δ 11.76 (br s, 1 H), 9.57 (s, 1H), 8.41 (d, J=2 Hz, 1H), 8.28 (d, J=5 Hz, 1H), 7.86 (d, J=2 Hz, 1H), 7.79 (d,J 9 Hz, 2H), 7.62 (d,J=9 Hz, 2H), 7.06 (d, J=5 Hz, 1H), 6.88 (brs, 2H), 3.74 (br s, 2H), 3.04 (m, 4H), 2.47 (m, 8H), 2.25 (s, 3H), 1.51 (m, 4H); ³C NMR (CDCl₃, 100 MHz) δ 165.7; 159.3; 151.0; 145.1; 142.7; 142.6; 135.5; 134.8; 134.5; 134.0; 128.7; 126.7; 124.2; 123.6; 110.4; 59.1; 54.2; 53.9; 46.1; 45.9; 23.6; MS (ES) 7m/z 536

### Example 159

### 3-Amino-6-(4-{[(2-(dimethylamino)ethyl](ethyl)amino]carbonyl}phenyl)-N-pyridin-3-ylpyrazine-2-carboxamide

Triethyl amine (33.2 mg, 0.255 mmol) in N,N-dimethylformamide (0.1 mL) was added to a solution of 4-(5-amino-6-[(pyridin-3-ylamino)carbonyl]pyrazin-2-yl)₆enzoic acid (52.9 mg, 0.15 mmol) and O-(benzotriazol-7-yl)-N,N,N′,N′-tetramethyluronium hexafluorophosphate (0.18 mmol) in N,N-dimethylformamide (8.5 mL). N″-Ethyl-N,N-dimethylethane-1,2-diamine (17.4 mg, 0.15 mmol) in N,N-dimetbylformamide (0.33 mL) was added and the mixture was shaken at room temperature for 24 h. Most of the solvent was removed and the crude reaction mixture was dissolved in dimethyl sulfoxide (1 mL) and purified by chromatography with acetonitrile/water (5:95 increasing to 0.95:5 for 12 minutes, XTerra C8-column 19×100 mm). The product was further purified by a second chromatography with acetonitrile/water (10:90 increasing to 60:10 in 13 minutes, XTerra C8S-column 19×300 mm) to give 8 mg (12% yield) of the title compound: MS (ES) m/z 434

The following Examples, 160-175, were synthesized as described for Example 159:

### Example 160

### 3-Amino-6-(4-{[[3-(dimethylamino)propyl](methyl)amino carbonyl}phenyl)-N-pyridin-3-ylpyrazine-2-carboxamide

Starting material: N,N,N′-trimethylpropane-1,3-diamine, yield 25%: MS (ES) m/z 434 (M⁺1+1)

### Example 161

### 3-Amino-6-[4-({[3-(dimethylamino)propyl]amino}carbonyl)phenyl]-N-pyridin-3-ylpyrazine-2-carboxamide.

Starting material: N,N,-dimethyl-1,3-propanediamine, yield 5%: MS (ES) m/z 420 (M⁺+1).

### Example 162

### 3-Amino-N-pyridin-3-yl-6-(4-{((2-pyrrolidin-1-ylethyl)amino]carbonyl}phenyl)pyrazine-2-carboxamide

Starting material: 2-pyrrolidin-1-yl-ethylamine, yield 29%: MS (ES) m/z 432 (M⁺+1).

### Example 163

### 3-Amino-N-pyridin-3-yl-6-(4-{[(3-pyrrolidin-1-ylpropyl)amino]carbonyl}phenyl)pyrazine-2-carboxamide

Starting material: 3-pyrrolidin-1-yl-propylamine, yield 14%: MS (ES) m/z: 446 (M⁺+1).

### Example 164

### 3-Amino-6-{4-[(4-methyl-1,4-diazepan-1-yl)carbonyl]phenyl}-N-pyridin-3-ylpyrazine2-carboxamide

Starting material: 1-methyl-[1,4]diazepane, yield 18%: MS (S) m/z 432 (M⁺+1).

### Example 165

### 3-Amino-6-(4-{[methyl(1-methylpyrrolidin-3-yl)amino}carbonyl]phenyl)-N-pyridin-3-ylpyrazine-2-carboxamide

Starting material: methyl-(1-methylpyrrolidin-3-yl)amine, yield 36%: MS (ES) m/z 432 (M⁺+1).

### Example 166

### 3-Amino-6-[4-({[2-(dimethylamino ethyl]amino}carbonyl)phenyl]-N-pyridin-3-ylpyrazine-2-carboxamide

Starting material: N,N-dimethylethylenediamine₁ yield: MS (ES) m/z 406 (M+I).

### Example 167

### 3-Amino-6-[4-({[2-(dimethylamino)-1-methylethyl]amino}carbonyl)phenyl]-N-pyridin-3-ylpyrazine-2-carboxamide

Starting material: N¹,N¹-dimethylpropane-1,2-diamine, yield 39%: MS (ES) m/z 420 (M⁺+1).

### Example 168

### 3-Amino-6-(4-{[3-(dimethylamino)pyrrolidin-1-yl]carbonyl}phenyl)-N-pyridin-3-ylpyrazine-2-carboxamide:

Starting material: dimethylpyrrolidin-3-ylamine, yield 41%: MS (ES) m/z 432 (M++I).

### Example 169

### 3-Amino-6-[4-(}[(1-ethylpyrrolidin-2-yl)methyl]amino}carbonyl)phenyl]-N-pyridin-3-ylpyrazine-2-carboxamide

Starting material: 2-(aminomethyl)-1-ethylpyrrolidine, yield 7%: MS (ES) m/z 446 (M⁺+1)”

### Example 170

### 3-Amino-6-[4-({[3-(4-methylpiperazin-1-yl)propyl]amino}carbonylyphenyl]-N-pyridin-3-ylpyriazine-2-carboxamide

Starting material: 3-(4-methyl-piperazin-1-yl)-propylamine, yield 23%: MS (ES) m/z 476

### Example 171

### 3-Amino-6-(4-{[methyl(1-methylpiperidin-4-yl)amino]carbonyl}phenyl)-N-pyridin-3-+ylpyrazine-2-carboxamide

Starting material: methyl-(1-methylpiperidin-4-yl)amine, Yield 27%: MS (ES) m/z 446

### Example 172.

### 3-Amino-6-(4-}[(2-pipendin-1-ylethyl)amino]carbonyl}phenyl)-N-pyridin-3-ylpyrazine-2-carboxamide

Starting material: 2-piperidin-1-ylethylamine, yield 5%: MS (ES) m/z 446 (M⁺+1).

### Example 173

### 3-Amino-6-(4-{[(-ethylpiperidin-3-yl)amino]6arbonyl]phenyl)-N-pyridin-3-ylpyrazine-2-carboxamide

Starting material: 1-ethylpiperidin-3-ylamine, yield 8%: MS ( ) m/z 446 (M⁺+1).

### Example 174

### 3-Amino-6-[4-({[2-(1-methylpyrrolidin-2-yl)ethyl]aminocarbonyl)phenyl]-N-pyridin-3-ylpyrazine-2-carboxamide

Starting material: 2-(1-methylpyrrolidin-2-yl)ethylamine yield 30%: MS (ES) mni/z 446

### Example 175

### 3-Amino-N-pyridin-3-yl-6-{4-[(4-pyrrolidin-1-ylpiperidin-1-yl)carbonyl]phenyl}pyrazine-2-carboxamide

Starting material: 4-pyrrolidin-1-ylpiperidine, yield 38%: MS (ES) m/z 472 (M⁺+1).

### Example 176

### 4-Amino-4′-[(4-methylpiperazin-1-yl)sulfonyl]-N-pyridin-3-yl-1,1′-bipbenyl-3-carboxamide

4-[(4-Methyl-1-piperazine-1-yl)sulfonyl)phenylboronic acid (0.06 g, 0.20 mmol), 2-amino-5-bromo-N-(3-pyridinyl)benzamide (0.155 g, 0.54 mmol), Na₂CO₃ (0.065 g, 0.62 mmol), and Pd(dppf)Cl₂ (4 mg, 0.006 mmol) were suspended in ethylene glycol dimethyl ether/water (2.6:0.6 mL) and heated in a microwave oven at 160° C. for 10 min. Silica was added and the solvent was evaporated. Purification by column chromatography using methylene chloride/methanol, (95:5), as the eluent gave 58 mg (63% yield) of the title compound: ¹H NMR (DMSO-d₆) δ 10.34 (s, 1H), 8.86 (d, J=5 Hz, 1H), 8.30 (m, 1H), 8.12 (m, 1H), 8.06 (d, J=2 Hz, 1H), 7.94 (d, J=9 Hz, 2H), 7.74 (d, J=9 Hz, 2H), 7.68(dd, J=9, 2 Hz, 1H), 7.39 (dd, J=8,4 Hz, 1H), 6.89 (d, J=9 Hz, 1H), 6.70 (brs, 2H), −2.89 (m, 4H), 2.35 (m, 4H), 2.13 (s, 3H); ¹³C NMR (DMSO-d₆) δ 156.4, 144.5, 144.4, 142.3, 135.7, 131.8, 131.0, 128.2, 127.7, 127.7, 123.1, 124.3, 123.5, 117.1, 114.5,53.5, 45.8, 45.3; MS (ESP) nVz 452 (M⁺1).

### Example 177

### 3-Amino-6-{2,5-difluoro-4-[(4-methylpiperazin-1-yl)sulfonyl]phenyl}-N-pyridin-3-ylpyrazine-2-carboxamide hydrochloride

Triisopropylborate (1.95 mL, 8.4 mmol) was added to a solution of 1-[(4-bromo-2,5-difluorophenyl)sulfonyl]0.4-methylpiperazine (1.0 g, 2.8 mmol) in anhydrous tetrahydrofuran (15 mL) at −78 aC under an atmosphere of nitrogen followed by dropwise addition of n-butyllithium (5.0 mL, 8.0 mmol) over 30 min. The resulting mixture was stirred at −78° C. for 2 h, HC (3 M aq, 4.7 mL, 14.1 mmol) was added, and the reaction mixture was allowed to warn to room-temperature. Sodium carbonate (3 g, 28.3 mmol) was added followed by the addition of 3-amino-6-bromo-N-pyridin-3-ylpyrazine-2-carboxamide (0.585 g, 1.99 mmol) and Pd(dppf)Cl₂ (80 mg, 0.10 mmol). The resulting mixture was heated at 70° C. for 16 h. Silica was added, the solvent was evaporated and the crude mixture was purified by column chromatography using methylene chloride/methanol, (95:5), to give 0.55:g (57% yield) of the base as a pale yellow solid: H. NMR (DMSO-d₆) δ 10.63 (s, 1H), 8.94 (s, 1H), 8.81 (s, 1H), 8.57 (m, 1H), 8.38 (m, 1 H), 8.17 (m, 1H), 8.03 (br s, 2H), 7.71 (m, 1H), 7.44 (m, 1H), 3.13(br s, 4H), 2.38 (br s, 4H), 2.15 (s, 3H); MS (TSP) m/z 491 (M⁺+1) HCl in diethyl ether (1 M, 0.81 mL) was added to a solution of the base (0.096 g, 0.21 mmol) in methylene chloride/methanol, (0.95:0.05, 8 mL). The yellow precipitate was filtered off, washed with diethyl ether and dried in vacuo to give 102 mg (99% yield) of the title compound as a yellow solid: ¹H (D₂O) δ 9.37 (d, J=2 Hz, 1H), 8.73 (s, 1H), 8.63 (m, 1H), 8.56 (d, J=6 Hz, 1H), 8.08 (dd, J=1.1, 6 Hz, 1H)802 (dd,J9,6 Hz, 1H), 7.73 (dd, J=10, 6 Hz, 1H), 4.05 (m, 2H), 3.63 (m, 2H), 3.27 (m, 2H), 3.16 (m, 2H), 2.93 (s, 3H); MS (TSP) m/z 491 I(M⁺+1).

The following Examples, 178-206, were synthesized as described for Example 177:

### Example 178

### 3-Amino-6-[3-fluoro-4-[(4-methylpiperazin-1-yl)sulfonyl]phenyl}-N-pyridin-3 ylpyrazine-2-carboxamide hydrochloride

Starting material: 1-[(4-bromo-2-fluoropheny])sulfonyl]4methylpiperazine. Yield: 49% o of the base: ¹H NMR (CDCl₃) δ9.88 (s, 1H), 8.83 (s, 1H), 8.70 (s, 1H), 8.36 (m, 1H), 8.26 (m, 1H), 7.90 (m, 1H), 7.79 (m, 2H), 7.37 (m, 1H), 3.36 (br s, 4H), 2.76 (s, 3H), 2.62 (br s, 4H); Ms (TSP) m/z 472 (M⁺+1). Hydrochloride, yield 93%: ¹H NMR (D₂O) δ 9.40 (d, J=2 Hz, 1H), 8.68 (s, 1H), 8.63 (m, 1H), 8.53 (m, 1H), 8.03 (m, 1H), 7.95 (dd, J=12, 1 Hz, 1H), 7.89 (dd, J=8, 3 Hz, I H), 7.80 (m, 1H), 3.96 (m, 2H), 3.55 (m, 2H), 3.20 (m, 2H), 3.04 (m, 2H), 2.86 (s, 3H); MS (TSP) m/z 472 (M⁺+1).

### Example 179

-3-Amino-6-{3-methyl-4-[(4-methylpiperazin-1-yl)sulfonyl]phenyl}-N-pyridin-3-ylpyrazine-2-carboxamide hydrochloride

Starting material: 1-[(4-bromo-2-methylphenyl)sulfonyl)₄methylpiperazine. Yield 62% as the base: ¹H NMR (CDCl₃) δ 9.86,(s, 1H), 8.85 (s, 1H), 8.74 (s, 1H), 8.45 (d, J=5 Hz, 1H), 8.30 (dd, J=8, I.Hz, 1H), 8.02 (d, J=8 Hz, 1H), 7.83 (d, J=8 Hz, 1H), 7.82 (s, 1H), 7.37 (dd, J 8, 5 Hz, 1H), 3.34 (br s, 4H), 2.74 (s, 3H), 2.62 (br s, 4. H), 2.39 (s, 3 H); ¹³C NMR (CDCl₃) 8164.4, 154.8, 146.0, 145.8, 141.9, 140.3, 139.1, 138.7, 135.4, 134.2, 131.4, 130.0, 127.3, 124.6, 124.0, 123.4, 54.3, 45.8, 45.0, 21.4; MS (TSP) mn/z 468 (M⁺+1). Hydrochloride, yield 99%: ¹H NMR (D₂O) δ 9.32 (d, J=2 Hz, 1H), 8.56 (m, 2H), 8.49(s, 1H), 8.02 (dd, =8, 6 Hz, 1H), 7.75 (m, 2H), 7.66 (d, J=8 Hz, 1H), 3.84 (m, 2H), 3.58 (m, 2H), 3.14 (m, 4H), 2.90 (s, 3H), 2.44 (s, 3H); ¹³C NMR (D₂O) δ 164.9, 153.9, 145.3, 139.7, 139.2, 137.5, 137.1, 137.0, 133.3, 132.6, 131.0, 129.4, 128.0, 123.6, 123.1, 53.1, 43.3, 42.6, 20.3; MS (TSP) m/z 468 (M⁺+1).

### Example 180

### 3-Amino-6-(2-[(4-methylpiperazin-1-razine-2carboxamide hydrochloride

Starting material: 1-[(2-bromophenyl)sulfonyl]-4-methylpiperazine. Yield 29% of the base: ¹H NMR (CDCl₃) δ 10.43 (s, 1H), 8.94 (s, 1H), 8.47 (m, 1H), 8.35 (m, 2H), 7.98 (m, 1-H), 7.69 (m, 1H), 7.60 (m, 2H), 7.29 (m, 1H), 3.27 (br s, 4H), 2.40 (br s, 4H), 2.28 (s, 3 H); ³C NMR (CDCl₃) δ 164.7, 154.1, 146.6, 145.3, 141.8, 140.1, 137.9, 136.9,135.1, 133.0, 132.0, 129.6, 129.1, 126.7, 124.1, 123.8, 54.5, 45-9, 45.3; MS (TSP) m/z 454 (M⁺+1) Hydrochloride, yield 99%: ¹H NMR (D₂O) δ 9.36 (s, 1H), 8.51 (m, 3H), 7.99 (m, 2H), 7.85 (m, 1H), 7.72 (m, 2H), 3.73 (,2H), 3.51 (m, 2H), 3.15 (m, 2H), 3.02 (m, 2H), 2.88 (s, 3H); ¹³C NMR (D₂O) δ 165.4, 154.1, 147.6, 139.6, 137.9, 137.3, 136.3, 136.1, 135.3, 134.7, 132.9, 132.8, 130.3, 129.9, 128.0, 123.2, 53.3, 43.3, 42.7; MS (TSP) mn/z 454

### Example 181

### 3-Amino-6-{3-[(4-methylpiperazin-1-yl)sulfonyl]phenyl}-N-pyridin-3-ylpyrazine-2-carboxamide hydrochloride

Starting material: 1-[(3-bromophenyl)sulfonyl]-4-methylpiperazine. Yield 63% as the base:

¹H NMR (CDCl₃) δ 9.85 (s, 1H), 8.82 (d, J=8 Hz, 1H), 8.71 (s, 1H), 8.43 (d, J=4 Hz, 1H), 8.28 (m, 1H), 8.22 (s, 1H), 8.12 (d, J=8 Hz, 1H), 7.78 (d, J=8 Hz, 1H), 7.69 (t, J=8 Hz, 1H), 7.35 (m, 1H), 3.26 (ir s, 4H), 2.72 (br s, 4H), 2.42 (s, 3H); ³C NMR (CDCl₃) δ 164.4, 154.7, 145.8, 145.5, 141.7, 13.8.7, 137.4, 136.5, 134.3, 130.2, 130.1, 127.8, 127.3, 124.9, 124.5, 124.0, 54.1,45.9, 45.7; MS (TSP) m/z 454 (M⁺+1).

Hydrochloride, yield 84%; ¹H NMR (D20) δ 9.33 (d, i=2 Hz, 1H), 8.62 (s, 1H), 8.55 (m, 2H), 8.23 (m, 1H), 8.16 (s, 1H), 8.03 (m, 1H), 7.67 (m, 2H), 3.93 (m, 2H), 3.58 (m, 2H), 3.23 (m, 2H), 2.87 (s. 3H), 2.83 (m, 2H); ¹³ C NMR (D₂O) 6165.2, 154.1, 145.3, 137.7, 137.6, 137.2, 137.0, 136.8, 135.1, 132.8, 131.1, 131.0, 128.0, 127.7, 124.0, 123.8, 53.0, 43.5,43.2; MS (TSP) m/z 454 (M⁺+1).:

### Example 182

### 3-Amino-6-{2-methyl-4-[(4-methylpiperazin-1-yl)sulfonyl]phenyl}-N-pyridin-3-ylpyrazine-2-carboxamide hydrochloride

Starting material: 1-[(4-bromo-3-methylphenyl)sulfonyl]-4-methylpiperazine. Yield 74% as the base: ¹H NMR (CDCl₃) δ 9.76 (s, 1H), 8.80 (s, 1H), 8.39 (s, 2H), 8.23 (d, J=8 Hz, 1H), 7.67 (m, 2H), 7.55 (d, J=8 Hz, 1H), 7.31 (m, 1H), 3.16 (br s, 4H), 2.64 (br s, 4-30H), 0.48 (s, 3H), 2.37 (br s, 3H); ¹³C NMR (CDCl₃) δ 164.2, 154.1, 147.5, 145.6, 141.5, 140.8, 140.5, 137.6, 134.7, 134.1, 130.1, 126.9, 125.7, 124.1, 123.6, 53.8, 45.6, 45.3, 20.8. Hydrochloride, yield 95%: ¹H NMR (DzO) δ 9.42 (d, J=2 Hz, 1H), 8.61 (m, 1H), 8.56 (m, 1. H), 8.43 (s, 1H), 8.03 (dd, J=8, 6 Hz, 1H), 7.78 (d, J=8 Hz, 2H), 3.96 (m, 2H), 3.61 (m, 2H), 3.26 (m, 2H), 2.91 (s,3H), 2.86 (m, 2H), 2.47 (s, 3H); ³C NMR (D20) 165.7, 153.9, 147.8, 141.6, 140.5, 139.0, 137.9, 137.4, 137.0, 133.8, 133.2, 131.0, 130.1, 128.0, 125.6, 43.9, 43.1, 20.2; MS (TSP) m/z 468 (M⁺+1).

### Example 183

### 3-Amino-6-[4-({[2-(dimethylamino)ethyl]amino}sulfonyl)-3-(trifluoromethoxy)phenyl]-N-pyridin-3-ylpyrazine-2-carboxamide hydrochloride

Starting material: 4-bromo-N-[2-(dimethylamino)ethyl]-2-(trifluoromethoxy)benzene-sulfonamide. Yield 56% as the base: ¹H NMR (DMSO-d₆) δ 10.67 (s, 1H), 9.07 (s, 1H), 8.95 (d, J=2 Hz, 1H), 8.42 (m, H), 8.35 (m, 2H), 8.20 (m, IH), 7.99 (d, J=8 Hz, I H), 7.93 (br s, 2H), 7.79 (br s, 1H), 7.45 (m,₁H),2.99 (t,J-=7 Hz, 2H), 2.28 (t, J=7 Hz, 2H), 2.07 (s, 6H); ¹³C NMR (DMSO-d₆) δ 164.8.154.9, 145.8, 145.6,145.5, 145.1, 142.8, 141.7, 135.3, 134.6, 132.6, 130.3, 128.2, 124.3, 24.1, 123.5, 121.3, 118.7, 118.3, 58.2,44.9; MS (TSP) nz/z 526 (M+11). Hydrochloride, yield 99%: ¹H NMR (D2O) δ 9.84 (br s, 1H), 8.64 (s, 1H), 8.56 (d, J=6 Hz, 1H), 8.45 (m, 1H), 7.99 (m 2H), 7.94 (m, 1H), 7.87 (d, J=8 Hz, 1H), 3.33 (s, 4H), 2.95 (s, 6H; ¹³C N (D₂O) δ 162.9, 152.4, 144.5, 143.7, 140.3, 135.6, 135.4, 134.2, 134.0, 130.9, 129.7, 127.2, 125.9, 122.0, 121.4, 119.7, 115.3, 54.8, 41.3, 35.9; MS (TSP) m/z 526 (M⁺+1).

### Example 184

3-Amino-6-[4-{[[2-(dimethylamino)ethyl](ethyl)amino]sulfonyl}-3-(trifluoromethoxy)phenyl]-N-pyridin-3-ylpyrazine-2-carboxamidehydrochloride

Starting material: 4-bromo-N-[2-(dimethylamino)ethyl]-N-ethyl-2-(trifluoromethoxy)benzenesulfonamide. Yield 87% as the base: ¹H NMR (CDCl₃) δ 9.79 (s, 1H), 8.82 (d, J=2 Hz, 1H), 8.76 (s, 1H), 8.44 (m, 1H), 8.25 (m, 1H), 8.16 (d, J=8 Hz, 1H), 7.96 (m, 1H), 7.87 (dd, J=8, 1 Hz, 1H), 7.37 (dd, J=85 Hz, 1H), 3.55 (m, 2 H), 3.40 (q, J=7 Hz, 2H), 2.72 (m, 2H), 2.39(S.6H)_{z 1.15} (t, J=:₇ HZ,₃H); ¹³C NMR (CDCl₃) δ 164.0, 155.1, 146.8, 146.0, 145.6, 142.0, 141.6, 137.0, 134.1, 132;7, 132.1, 126.9, 124.7, 124.5, 124.0, 122.9, 121.9, 119.3, 117.0, 58.0, 45.2, 44.6, 43.5, 14.4; MS (TSP) m/z 554 (M⁺+1). Hydrochloride, yield 91%: ¹H NMR (D₂O) δ 9.42 (d, J=2 Hz, 1H), 8.63 (s, 1H), 8.60 (d, J=6 Hz, H), 8.50 (m, 1H), 8.06 (m, 1H), 7.95 (m, 2H), 7.89 (d, J=8 Hz, 1H), 3.74 (t, J=6 Hz, 2H), 3.42 (t, J=6 Hz, 2H), 3.33 (q, J=7 Hz, 2H), 2.98 (s, 6H), 0.97 (t, J=7 Hz, 3H); i³C NMR (D₂O) δ 162.7, 152.1, 144.0, 143.4, 140.0, 135.3, 134.9, 134.4, 133.6, 130.3, 130.1, 127.1, 125.8, 121.7, 121.2, 114.8, 53.5,41.3, 41.1, 11.3, 10.2; MS (TSP)m/z 554 (M⁺+1).

### Example 185

### tert-Butyl 2-{[(4-[5-amino-6-[(pyridin-3-ylamino)carbonyl]pyrazin-2 yl}phenyl)sulfonyl]-(tert-butoxycarbonyl)amino}ethylcarbamate

Starting material: tert-butyl 2-(([4-bromo-2-(trifluoromethoxy)phenyl]sulfonyl}-(tert-butoxycarbonyl)armino)ethylcarbamate. The product was used in the next step without further analysis.

### Example 186

### 3-Amino-6-[4-[(4-methylpiperazin-1-yl)sulfonyl]-2-(trifluoromethyl)phenyl]-N-pyridin-3-ylpyrazine-2-carboxamide hydrochloride

Starting material:1-[([4-bromo-3-(trifluoromethyl)pbenyl]sulfonyl} lmethylpiperazine.

Purification on a reversed phase column (XTerra C8 19x300 mm) using a water/acetonitrile gradient as the eluent gave the base in 3% yield: ¹H NMR (CDCl₃) δ 9.79 (br s, 1H), 8.81 (br s, 1H), 8.44 (s, 1H), 8.41 (m, 1H), 8.21 (m, 2H), 8.03 (dd, J=8, 2 Hz, 1H), 7.74 (m, H), 7.32 (dd, J=8, 5 Hz, 1H), 3.19 (m, 4H), 2.63 (m, 4H), 2.37 (br s, 3H); MS (TSP) m/z 522 (M⁺+1). Hydrochloride, yield 99%: ¹H NMR (D20, 400 MHz) δ 9.18 (br S, 1H), 8.47 (m, 2H), 8.41 (m, 1H), 8.28 (m, 1H), 8.17 (m, 1H), 7.94 (d, J=8 Hz, 1H), 7.88 (m, 1H), 4.04 (m, 2H), 3.64 (m, 2H), 3.49 (m, 1H), 3.30 (m, 2H), 3.12 (m, 1H), 2.92 (s, 3H); MS (TSP) m/z 522.0 (M⁺+1).

### Example 187

### 3-Amino-6-[4-[2-(dimethylamino)ethoxy]phenyl]-N(3-pyridinyl)-2-pyrazine-carboxamide hydrochloride

Starting material: N-[2-(4-bromophenoxy)ethyl]-N,N-dimethylami.ne (described in Ruenitz, P., et al, J. Med. Chem. 1982, 25, 1056-1060). Yield 19% of the base: ¹H NMR (DMSO-d6) δ 10.56 (s, 1H), 8.97 (s, 1H), 8.88 (s, 1H), 8.34 (d, J=3 Hz, 1H), 8.21 (m, 1H), 8.17 (d, J=9 Hz, 21H), 7.58 (br s, 21H), 7.42 (dd, J=8, 4 Hz, 1H), 7.03 (d, J=9 Hz, 2H), 4.11 (t, J=6 Hz, 2H), 2.64 (t, J=6 Hz 2H), 2.22 (s, 6H); ³CNMR (DMSO-d₆) δ 165.3, 158.8, 153.9, 145.0, 144.4, 143.0, 138.9, 134.7, 128.4, 127.2, 123.5, 123.0, 115.7, 114.6, 65.8, 57.7, 45.6; MS (EI) m/z 379 (M⁺+1). Hydrochloride: yield 45%.

### Example 183

3-Amino-6-[4-[2-(4-morpholinyl)ethoxy]phenyl]-N-(3-pyridinyl)-2-pyrazinecarboxamide hydrochloride

Starting material: 4-[2-(4-bromophenoxy)ethyl]morpholine (described in Lednicer, D., et al, J. Med. Chem. 1965, 8, 52-57). Yield 20% of the base: ¹H NMR (DMSO-d₆) δ 10.55 (s, 1H), 8.99 (br s, 1H), 8.88 (s, 1H), 8.4 (m, 1H), 8.22 (d, J=8 Hz, 2H) 8.12 (d, J=9 Hz, 2H), 7.56 (br s, 21H), 7.51 (d, J=9 Hz, 1H), 7.41 (dd, J=8, 5 Hz, 1H), 4.16 (t, J=0.6 Hz, 2H), 4.08 (t, J=6 Hz, 1H), 3.58 (in, 6H), 2.72 (m, 3H); ¹³C NMR (DMSO-d₆) δ 165.2, 158.7, 157.5, 153.8, 144.9, 144.3, 143.0, 138.8, 132.3, 128.3, 127.1, 123.4, 123.0, 114.8, 114.6, 66.1, 65.3, 56.9, 53.6; MS (EI) m/z 421 (M++I). Hydrochloride: yield 46%.

### Example 189

### 3-Amino-6-[4-[([2-(dimethylanuno)ethyl]methylamino]carbonyl]phenyl]-N-(3-pyridinyl)-2-pyrazinecarboxamide hydrochloride

Starting material: 4-bromo-N-[2-(dimethylamino)ethyl]-N-methylbenzamide. Yield 20% of the base: ¹H NMR (CDCl₃) δ 10.59 (s, 1H), 8.98 (s, 2H), 8.36 (dd, J=4, 1 Hz, 1H), 8.30 (d, J=8 Hz, 2H), 8.21 (m, 1H), 7.73 (br s, 2H), 7.49 (d, J=9 Hz, 2H), 7.42 (dd, J=8, 4 Hz, 1H), 3.32 (br s, 4H), 2.96 (br s, 3H), 2.23 (br s, 3H), 1.99 (br s, 3H); ¹³C NMR (CDCl₃) δ 139.4, 154.2, 145.5, 145.3, 141.5, 134.1, 127.0, 125.5, 124.0, 123.8, 109.5, 76.7, 58.4, 50.8, 45.5, 29.7, 22.7, 18.4; MS (ED m/z 420 (M⁺+1). Hydrochloride, yield 28%: ¹H NMR (D20) δ 9.34 (s, 1H), 9.33 (s, 1H), 8.65 (s, 1H), 8.58 (d, J=0.9 Hz, 1H), 8.50 (d, J=6 Hz, 1H), 7.99 (d, J=8 Hz, 2H), 7.52 (d, J=8 Hz, 2H), 3.92 (t, J=6 Hz, 2H), 3.48 (t, J=7 Hz, 2H), 3.06 (s, 3H), 3.00 (s, 6H).

### Example 190

### 3-A 2-no-6-[4-[2-(4-methyl-1-piperazinyl)ethoxyphenyl]-N-(3-pyridinyl)-2-pyrazinecarboxamide

Starting material: 1-[2-(4-bromophenoxy)ethylj4-methylpiperazine (described in Ide, et al., J. Am. Chem. Soc., 1954, 76, 1122-1125). Yield 66% of the base: ¹H NMR (DMSO-d₆) 10.54 (s, 1H), 8.97 (d, J=2 Hz, 1H), 8.88 (s, 1H), 8.34 (dd, J=5, 2 Hz, 1H), 8.21 (m, 11H), 8.16 (d,J-9 Hz, 2H), 7.56 brs, 2H), 7.42 (dd, J=8, 5 Hz, 1H), 7.03 (d, J=9 Hz, 2H), 4.14 (t, J=6 Hz, 2H), 2.72 (t, J=6 Hz, 2H), 2.57 (brs, 8H), 2.31 (s, 3H); MS (ES) m/z 434 (M⁺+1). Hydrochloride, yield 92%: ¹H NMR (D₂O) δ 9.4 (s, 1H), 8.66 (s 1H), 8.63 (d, J=9 Hz, 1H), 8.55 (d, J=6 Hz, 1H), 8.05 (dd, J=8, 6 Hz, 1H), 7.94 (d, J=9 Hz, 2H), 7.63 (d, J=9 Hz, H), 7.13 (d, J=9 Hz, 2H), 4.43 (t, J=S Hz 2H), 3.62 (br s, 10H), 3.0 (s, 3H); ¹³C NMR (D₂) δ 166.2, 158.8, 154.0, 145.4, 140.8, 138.3, 137.6, 137.4, 133.8, 129.7, 128.4, 128.0, 124.0, 115.9, 62.9, 56.7, 51.2, 50.2, 43.9.

### Example 191

### 3-Amino-6-[4-[[[2-(4-morpholinyl)ethyl]amino]carbonyl]phenyl]-N-(3-pyridinyl)-2-pyrazinecarboxamide

Starting material: 4-bromo-N-(2-morpholin-4-ylethyl)benzamide described in Rafli, H., et al., Life Sci. 1996, 58, 1159-1170, yield 1% as the base: ¹H NMR (DMSO-d₆) δ 10.61 (s, 2H), 9.02 (s, 1H), 8.96 (d, J=2 Hz, 1H), 8.49 (m, 1H), 8.34 (d, J=9 Hz, 2H), 8.20 (m, 1H), 7.94 (d, =9 Hz, 2H), 7.78 (br s, 2H), 7.44 (dd, J=8, 4 Hz, 1H), 3.57 (t, J=5 Hz, 4H), 3.40 (m, 4H), 2.42 (m, 4H); MS (ES) m/z 448 (M⁺+1).

### Example 192

### 3-Amino-6-[4-[(1-methyl-3-pyrrolidinyl)oxy]phenyl]-N-(3-pyridinyl). 2-pyrazinecarboxamide

Starting material: 3-(4-bromophenoxy)-1-methylpyrrolidine. Yield 79% of the base: ¹H NMR (DMSO-d₆) δ 10.54 (s, 1H), 8.97 (d, J=2 Hz, 1H), 8.87 (s, 1H), 8.34 (dd, J=5, 2 Hz, 1H), 3.21 (m, 1H), 8.15 (d, J=9 Hz, 2H), 7.56 (br s, 2H), 7.42 (dd, J=8, 5 Hz, 1H), 6.96 (d, J=9 Hz, 2H), 4.93 (m, 1H), 2.79 (m, 1H), 2.66 (m, 2H), 2.35 (m, 2H), 2.26 (s, 3H), 1.78 (m, 1H); 3C NMR (DMSO-d₆) δ 165.2, 157.6, 153.8, 144.9, 144.3, 142.9, 138.8, 134.6, 128.2, 127.2, 123.4, 123.0, 115.2, 76.7, 61.7, 54.6, 41.6, 32.4; MS (ES) m/z 391 (M⁺+1). Hydrochloride, yield 96%: ¹H NMR (DMSO-d₆) δ 11.12 (s, 1H), 9.42 (d, f=2 Hz, 1H), 8.96 (m, 2H), 8.69 (d, J=5 Hz, 1H), 8.22 (m, 2H), 8.08 (dd, J=9, 6 Hz, 1H), 7.08 (d, J=9 Hz, 2H), 5.25 (m, 1H), 3.97 (m, 1H), 3.65 (m, 2H), 3.17 (m, 2:H), 2.85 (m, 3H), 2.29 (m, 1H).

### Example 193

### 3-Amino-6-{2-fluoro-4-[(4-methylpiperazin-1-yl)sulfonyl]phenyl}-N-pyridin-3ylpyrazine-2-carboxamide hydrochloride

Starting material: 1-[(4-bromo-3-fluorophenyl)sulfonyl]-4-methylpiperazine. Yield 36% of the base: MS (ES) m/z 472 (M⁺+1).

Hydrochloride, yield 28%: ¹H NMR (DMSO-d₆) δ 10.58 (s, 1H), 8.96 (m, 1H), 8.78 (d, J=2 Hz, 1H), 8.53 (t, J=8 Hz, 1H), 8.35 (dd,J 4,2 Hz, 1H), 8.2 (m, 1H), 7.92 (brs, 2H), 7.68 (m, 2H), 7.42 (m, 1H), 2.97 (m, 4H), 2.37 (m, 4H), 2.14 (s, 3H); ³C NMR (DMSO-d₆) S 164.6, 160.2, 157.7, 154.4, 147.9, 145.0, 142.8, 135.8, 134.4, 132.8, 131.5, 128.3, 128.2, 124.8, 124.8, 123.6, 115.3, 53.4, 45.7, 45.2.

### Example 194

### 3-Amino-6-({5 fluoro-2-methyl-4-[(4-methylppiperazin-1-yl)sulfo nylophenyl}-N-pyridin-3-ylpyrazine-2-carboxamide hydrochloride

Starting material: 1-[(4-bromo-2-fluoro-5-methylphenylsulfonyl]4-methylpiperazine Purification by chromatography on silica gel using a gradient of ethyl acetatelheptane, (1:100), to ethyl acetate/methanol, (10:1), followed by formation of the hydrochloric salt in 3 ML of a methylene chloride/methanol mixture (v/v=3: 1) by the addition of 5 mL of hydrochlorid acid in diethyl ether (1 M) gave after washing with diethyl ether and drying 92 mg (48% yield) of the title compound: ¹H NMR (D₂O, 400 MHz) δ 9.41 (d, J=3 Hz, 1H), 8.62 (m, 1H), 8.56 (m, 1H), 8.39 (s, 1H), 8.04 (dd, J=9, 6 Hz, 1H), 7.72 (d, J=7 Hz, 1H), 7.53 (d, J=11 Hz, 1H), 4.00 (m, 2H), 3.62 (m, 2H), 3.24 (m, 2H), 3.10 (m, 2Hz 2.92 (s, 3H), 2.40 (s, 3H); MS (ES) m/z 486 (M⁺+1).

### Example 195

### 3-Amino-6-{2,5-dimethyl-4-[(4-methylpiperazin-1-yl)sulfonyl]phenyl}-N-pyridin-3-ylpyrazine-2-carboxamide hydrochloride

Starting material: 1-[(4-bromo-2,5-dimethylphenyl)sulfonyl]-4-methylpiperazine. Purification by chromatography on silica gel using a gradient of ethyl acetate/heptane, (1:100), to ethyl acetate/methanol, (10:1), followed by formation of the hydrochloric salt in 3 mL of a methylene chloride/methanol mixture (v/v-=3: 1) by the addition of 5 mL of hydrochlorid acid in diethyl ether (1 M) gave after washing with diethyl ether and drying 90 mg (48% yield) of the title compound: ¹H NMR (D₂O, 400 MHz) δ 9.40 (m, 1H), 8.59 (m, 1H), 8.55 (m, 1H), 8.36 (s, 1H), 8.03 (ddd, J=9, 6, 1 Hz, 1H), 7.78 (s, 1H), 7.50 (s, 1H), 3.90 (d, J=12 Hz, 2H), 3.60 (d, J=1 Hz, 2H), 3′.18 (quint, J=13 Hz, 4H), 2.93 (s, 3H), 2.52 (s, 3H), 2.38 (s, 3H); MS (ES) m/z 482 (M⁺+1).

### Example 196 —

### 3-Amino-6-[4-(2-piperidin-1-ylethoxy)phenyl]-N-pyridin-3ylpyrazine-2carboxamide hydrochloride

Starting material: 1-[2-(4-bromophenoxy)ethyl]piperidine (described in: Stauffer, S. R. et al, Bioorg. Med. Chem. 2001, 9, 151-162): ¹H NMR (DMSO-d₆, 300 MHz) δ 10.56 (s, 1H), 8.98 (d, J=2 Hz, 1H), 8.88 (s, 1H), 8.36 (dd, J=5, 1 Hz, 1H), 8.22 (m, 1H), 8.17 (d, J=9 Hz, 2H), 7.57 (brs, 2H), 7.43 (dd, J=8, 5 Hz, 1H), 7.04 (d, J=9 Hz, 2H), 4.13 (t, J=6 Hz, 2H), 2.67 (t, J=6 Hz, 2H), 2.44 (m, 4H), 1.50 (m, 4H)) 1.39 (m, 2H). Hydrochloride, yield 28%: ¹H NMR (DMSO-d₆, 300 MHz) δ 11.18 (s, 1H), 10.91 (m, 1H), 9.50 (d, J=2 Hz, 1H), 9.07 (s, 1H), 9.00 (d, J=9 Hz, 1H), 8.78 (d, J=5 Hz, 1H), 8.35 (d, J=9 Hz, 2H), 8.15 (dd, J=9, 6 Hz, 1H), 7.23 (d, J=9 Hz, 2H), 4.64 (r, 2H) 3.60 (m, 4H), 3.11 (m, 2H), 1,93 (m, 4H), 1.26 (m, 2H); ¹³C NMR (DMSO-d₆, 75 MHz) δ 165.7, 157.9, 153.9, 145.1, 138.9, 137.9, 136.5, 136.0, 133.2, 128.8, 127.4, 127.3, 122.1, 114.8, 62.5, 54.6, 52.6,22.2, 21.2; MS (ES) 419 (M⁺+1)

### Example 197

### 3-Amino-6-[4-(2-pyrrolidin-1-ylethoxy)phenyl]-N-pyridin-3-yl-pyrazine-2-carboxamide hydrochloride

Starting material: 1-[2-(4-bromophenoxy)ethylpyrrolidine (described in Penning, T. D. et al, J. Med. Chem. 2000, 43, 721-735). Hydrochloride: ¹HNMR (DMSO-d₆, 390 MHz) δ 11.26 (br s, 1H), 1.10 (s, 1H), 9.41 (d, J.=2 Hz, 1H), 8.95 (s, —H), 8.93 (d, J=7 Hz, 1 H), 8.68 (d, J=5 Hz, 1H), 8.23 (d, J=9 Hz, 2H), 8.07 (dd, J=9, 6 Hz, 1H), 7.11 (d, J=9 Hz, 2H), 4.44 (m, 2H), 3.58 (m, 4H), 3.12 (m, 2H), 1.94 (m, 4H); MS (ES) 405(M⁺+1).

### Example 198

### tert-Butyl 4[2-(4-{5-amino-6-[(pyridin-3-ylamino)carbonyl]pyrazin-2-yl}phenoxy)ethyl]piperazine-1-carboxylate

Starting materials: tert-butyl 4-[2-(4bromophenoxy)ethyl]piperazine-1-carboxylate, yield 70% as the base: ¹H NMR (DMSO-d₆, 300 MHz) δ 11.66 (s, 1H), 10.09 (d, J=2 Hz, 1H), 9.99 (s, 1H), 9.46 (m, 1H), 9.32 (m, 1H), 9.28 (d, J=0.9 Hz, 2H), 8.68 (s, 2H), 8.54 (dd, J=8, 5 Hz, 1H), 8.16 (d, J=9 Hz, 2,H), 5.26 (t, J=6 Hz, 2H), 4.42 (m, 4H), 3.85 (t, J=6 Hz, 2H), 3.57 (m, 4H), 2.50 (s, 9H); MS (ES).520 (M⁺+1).

### Example 199

### 3-Amino-6-{4-[(4-methylpiperazin-1-yl)carbonyl]phenyl}-N-pyridin-3-ylpyrazine-2-carboxamide hydrochloride

Starting material: 1-(4-bromobenzoyl)-4-methylpiperazine. Hydrochloride, yield 26%: ¹H NMR (DMSO-d₆, 300 MHz) δ 11.41 (br s, 1H), 11.11 (s, 1H), 9.40 (s, 1H), 9.06 (s, 1H), 8.90 (d, J=9 Hz, H), 8.69 (d, J=5 Hz, 1H), 8.38 (d, J=8 Hz, 2H), 8.06 (dd, J=9, 6 Hz, 1H), 7.59 (d, J=8 Hz, 2H), 3.39 (m, 4H), 3.13 (m, 2H), 2.77 (s, 3H), 2.50 (m,2 H); MS (ES) 418 (M⁺+1).

### Example 200

### tert-Butyl 4-[2-(4-{5-amino-6-[(pyridin-3-ylamino)carbonyl]pyrazin-2-yl]-2,5-difluorophenoxy)ethyl]piperazine-1-carboxylate

Starting material: tert-butyl 4-[2-(4-bromo-2,5-difluorophenoxy)ethyl]piperazine-1-carboxylate, yield 22% as the base: ¹H NMR (DMSO-d₆, 300 MHz) δ 10.57 (s, 1H), 8.95(d, J=2 Hz, 1H), 8,69 (d, J=2 Hz, 1H), 8.37 (dd, J=5, 1 Hz, 1H), 8.29 (dd, J=13, 8 Hz, 1 H), 8.17 (dd, J8, 1 Hz, 1H), 7.75 (s, 2H), 7.44 (dd, J8, 5 Hz, 1H), 7.35 (dd, J=13,7 Hz, 1H), 4.25 (t, J=6 Hz, 2H), 2.75 (t, J=6 Hz, 2H), 3.31 (m, 4H), 2.46 (m, 4H), 1.39 (s, 9H).

### Example 201

### 3-Amino-6-[2,5-difluoro-4-(2-morpholin-4-ylethox)phenyl]-N-pyridin-3-ylpyrazine-2-carboxamide hydrochloride

Starting material: 4-[2-(4-bromo-2,5-difluorophenoxy)ethyl]morpholine. Hydrochloride, yield 63%: ¹H NMR (DMSO-d₆, 300 MHz) δ 11.07 (s, 1H), 9.37 (d, J=2Hz, 1H), 8.86 (d, J=9 Hz, 1H), 8.70 (s, 1H), 8.68 (m, 1H), 8.33 (dd, J=12, 7 Hz, 1H), 8.02 (dd, J=8, 5 Hz, 1H), 7.77 (br s, 2H), 7.36 (dd, J=13, 7 Hz, 1H), 4.66 (m, 2H), 3.96 (m, 4H), 3.63 (m, 2H), 3.50 (m, 2H), 3.27 (m, 2H).

### Example 202

### 3 Amino-6-[2,5-difluoro-4-(2-pyrrolidin-1-ylethoxy)phenyl]-N-pyridin-0.3-ylpyrazine-2-carboxamide hydrochloride

Starting material: 1-[2-(4-bromo-2,5-difluorophenoxy)ethyl]pyrrolidine, yield 31%: ¹H NMR (DMSO-d₆,300 MHz) δ 10.88 (s, 1H), 10.74 (br s, 1H), 9.21 (d, J=2 Hz, 1H), 8.74 (d, J=2 Hz, 1H), 8.58 (m, 2H), 8.34 (dd, J=12, 7 Hz, 1H), 7.81 (dd, J=8, 5 Hz, 2 H), 7.41 (dd, J=13, 7 Hz, 1H), 4.54 (m, 2H), 3.64 (m, 4H), 3.13 (m, 2H), 2.04 (m, 2H), −1.90 (m, 2H); MS (ES)441(M⁺+1).

### Example 203

### 3-Amino-6-{2,6-dimethyl-4-(2-(4-methylpiperazin-1-yl)ethoxyhphenyl}-N-pyridin-3-ylprazine-2-carboxamide hydrochloride

Starting material: 1-[2-(4-bromo-3,5-dimethylphenoxy)ethyl]-4-methylpiperazine. Hydrochloride, yield 7%: ¹H NMR (DMSO-d₆, 300 MHz) δ 10.46 (s, 1H), 9.3.1 (s, 1H), 8.93 (d, J=2 Hz, 1H), 8.30 (m 1H), 8.21 (m, 2H), 762 (brs, 2H), 7.38 (m, 1H), 6.76 1 (s, H), 4.12 (m, 2H), 2.79 (m, 10H), 2.50 (s, 3H), 2.09 (s, 6H); MS (ES) 462 (M⁺+1).

### Example 204

### 3-Amino-6-{2-methyl-4-[2-(4-methylpiperazin-1-yl)ethoxy]phenyl}N-pyridin-3-ylpyrazine-2-carboxamide hydrochloride

Starting material:1-[2-(4-bromo-3-methylphenoxy)ethyl]4-methylpiperazine. Hydrochloride, yield 23%: ¹H NMR (DMSO-d₆, 300 MHz) δ 11.10 (s, 1H), 9.38 (d, J=2 Hz, 1H), 8.89 (d, J=9 Hz, 1H), 8.67 (d, J=5 Hz, 1H), 8.49 (s, 1H), 8.04 (dd, J9, 5 Hz, 1H), 7.52 (d, J=0.8 Hz, 2H), 7.01 (d,1H), 6.97 (m, 1H), 4.52 (m, 2H), 3.81 (m, 10H), 2.84 (s, 3H), 2.41 (s, 3H); MS (ES) 448 (M⁺+1).

### Example 205

### 3-Amino-6-{5-[(dimethylamino)sulfonyl]thien-2-yl}-N-pyridin-3-ylpyrazine-2-carboxamide

Starting material: 5-bromo-N,N-dimethylthiophene-2-sulfonamide. Purification by chromatography on silica gel using a gradient of ethyl acetate/heptane, (1:100), to ethyl acetate/methanol (1:1), gave 80 mg (28% yield) of the title compound as the base: ¹H NMR (DMSO-d₆, 400 MHz). 10.47 (s, 1H), 8.98 (m, 2H), 8.39 (m, 1H), 8.18 (m, 1H), 7.97 (m, 1H), 7.91 (m, 2H), 7.68 (m, 1H), 7.46 (m, 1H), 2.72 (s, 6H) ¹³CNMR (DMSO-d6, 100 MHz) 6164.4, 154.5, 147.6, 145.1, 144.5, 143.1, 133.7, 133.4, 133.2, 128.6, 123.9, 123.6, 123.4, 37.7; MS (ES) m/z 405.24 (M++).

### Example 206

### tert-Butyl 4-(5-{5-amino-6-[(pyridin-3-ylamino)carbonyl]pyrazin-2-yl}-2-furoyl)piperazine-1-carboxylate

Starting material: tert-butyl 4-(5-bromo-2-furoyl)piperazine-1-carboxylate. Purification by chromatography on silica gel using a gradient of ethyl acetate/heptane, (1:100), to ethyl acetate/methanol, (10:1), gave the title compound as the base, yield33%: ¹H NMR (CD₃OD, 400 MHz) δ9.02 (m, 1H), 8.74 (s, 1H), 8.33 (m, 2H), 7.48 (dd, J=8, 5 Hz, 1 H), 7.31 (d, J=4 Hz, 1H) 7.20 (d, J=4 Hz, 1H), 3.87 (m, 4H), 3.58 (m, 4H), 1.48 (s, 9H); MS (ES) m/z 494. (M⁺+1).

### Example 207

### 2-Amino-5-[4-[(4-methylpiperazin-1-yl)sulfonyl]phenyl)-N-pyridin-3-yinicotiamide hydrochloride

4-[(4-Methylpiperazin-1-yl)sulfonyl]phenylboronic acid (157 mg, 0.55 mmol), 2-amino-5-bromo-N-pyridin-3-ylnicotinamide (54 mg, 0.18 mmol), sodium carbonate (58 mg, 0.54 nmol), Pd(dppf)Cl₂×CH₂Cl₂ (7 mg, 0.01 mmol) were suspended in ethylene glycol dimethyl ether/water (2.5:0.6 mL) and heated in a microwave oven at 160° C. for 10 min. Silica was added and the solvent was evaporated. Purification by chromatography on a silica gel column using ethyl acetate to ethyl acetate/methanol, (10:1), as the eluent gave a product which was-further purified by reversed phase chromatography (water/acetonitrile/ammonium acetate gradient, column: XTerra C8 19x300 mm). After removal of the solvent, the residue was dissolved in methylene chloride. The organic layer was washed with an aqueous saturated sodium hydrogen carbonate solution and subsequently dried over sodium sulfate. Filtration and removal of solvent in vacuo gave an oil which was dissolved in 3 mL methylene chloride/methanol mixture (v/v=3:1).

Hydrochloric acid (5 mL, 1 M in diethyl ether) were added and the precipitate was washed with diethyl ether and dried in vacuo to give 50 mg (53% yield) of the title compound: ¹H NMR (D₂O, 400M{z) δ 9.35 (d, J,=2 Hz, 1H), 8.82 (d; J=2 Hz, 1H), 8.64 (ddd, J=9, 2, 1 Hz, 1H), 8.57 (m, 1H), 8.43 (d, J=2 Hz, 1H), 8.04 (m, 1H), 7.89 (m, 4H), 3.92 (d, J=14 Hz, 2H), 3.57 (d, J=13 Hz, 2H), 3.21 (m, 2H), 2.86 (s, 3H), 2.82 (m, 2H); ¹³C NMR (D₂O, 100 MHz) δ 163.2, 151.1, 140.6, 137.9, 137.7, 135.7, 135.5, 135.4, 131.7, 131.6, 126.7, 125.7, 125.5, 121.6, 112.9, 50.7, 41.2, 40.9; MS (ES) m/z 453 (M⁺+1).

The following Examples, 208-213, were synthesized as described for Example 207:

### Example 208

### 3Amino-6-y 4-[(4-Methylpiperazin-1-yl)sulfonyl]phenyl}-N-[4-(pyrrolidin-1-ylmethyl)pyridin-3-yl]pyrazine-2-carboxamide hydrochloride

Starting material: 4-[(4-methylpiperazin-1-yl)sulfonyl]phenylboronic acid and 3-amino-6-bromo-N-[4-(pyrrolidin-1-ylmethyl)pyridin-3-yl]pyrazine-2-carboxamide. Purification by chromatography on a silica gel column using ethyl acetate to ethyl acetate/methanol, (1:1), as the eluent followed by formation of the hydrochloric salt in 3 mL of a methylene chloride/methanol mixture (v/v=3: 1) by the addition of hydrochloride acid in diethyl ether (5 mL, 1 M) gave after washing with diethyl ether and drying 70 mg (23% yield) of the title compound: ¹H NMR (D₂O, 400 MHz) δ 9.01 (s, 1H), 8.79 (d, J=6 Hz, 1H), 8.75 (m, 1H), 8.15 (m, 3H), 7.83 (d, J=9 Hz, 2H), 4.68 (s, 2H), 3.90 (d, J=14 Hz, 2H), 3.56 (d, J=12 Hz, 2H), 3.39 (br m, 4H), 3.20 (t, J=12 Hz, 2H), 2.85 (s, 3H), 2.79 (m, 2H); 2.03 (br s, 4H); ¹³CNMR (D₂O, 100 MHz) δ 166.9, 154.5, 146.0, 143.8, 143.4, 142.7, 141.1, 138.4, 135.1, 133.6, 128.6, 127.8, 126.9, 124.4, 55.5, 53.1, 52.9, 43.5,43.1, 22.9; MS (ES) m/z 537 (M⁺+1).

### Example 209

### 3-Amino-6-[2,5-difluoro-4-(pyrrolidin-1-ylsulfonyl)phenyl]-N-(4-(2-pyrrolidin-1ylethyl)pyridin-3-ylpyrazine-2-carboxamide hydrochloride

Starting materials: 2,5-difluoro-4-(pyrrolidin-1-ylsulfonyl)phenylboronic acid and 3amino-6-bromo-N-[4-(2-pyrrolidin-1-ylethyl)pyridin-3-yl]pyrazine-2-carboxamide.

Purification by chromatography on a silica gel column using ethyl acetate/heptane, (10:1), to ethyl acetate/methanol, (1:1), as the eluent, followed by formation of the hydrochloric salt in 3 mL of a methylene chloride/methanol mixture (v/v=3:1) by the addition of hydrochloride acid in diethyl ether (5 mL,1 M) gave after washing with diethyl ether and drying 40 mg (35% yield) of the title coripound: ¹H NMR (D20, 400 MHz) δ 9.04 (s, 1H), 8.78 (d, J=2 Hz, 1H), 8.61 (d, J=5 Hz, 1H), 8.02 (dd, J=10, 6 Hz, 1H), 7.89 (d, J=6 Hz, 1H), 7.75 (dd, J=10, Hz, 1H), 3.59 (br m, 4H), 3.36 (m, 6H), 3.04 (m, 2H), 2.00(m, 2-H), 1.90 (m, 2H), 1.82 (mi, 4H); MS (ES) m/z 558 (M+t1).

### Example 210

### 3-Amino-6-[2,5-difluoro-4-(pyrrolidin-1-ylsulfonyl)phenyl]-N-[5-(3-pyrrolidin-1-ylpropyl)pyridin-3-yl]pyrazine-2-carboxamide hydrochloride

Starting materials: 2,5-difluoro-4-(pyrrolidin-1-ylsulfonyl)phenylboronic acid and 3-amino-6-bromo-N-[5-(3-pyrrolidin-1-ylpropyl)pyridin-3-ylpyrazine-2-carboxamide. The product mixture was filtered through C-8 reversed phase gel, using acetonitrile. The solvent was evaporated and the crude product was purified by reversed phase chromatography (column: XTerra C8 19x300 mm) using a water/acetonitrile gradient to give 21 mg, (20% yield).of the base. The base was dissolved in methylene chloride/methanol, (89:1, 5.0 mL) and cooled to, 0° C. HCl in diethyl ether (5 mL, 1 M) was added dropwise and the mixture was stirred for 30 min at 0’0 C. The precipitate was collected by filtration and washed with diethyl ether and dried to give 15 mg (12% yield) of the title compound: ¹H NMR (CD₃OD, 400 MHz) δ 9.42 (d, J=2 Hz, 1H),8.77 (d, J=2 Hz, 1H), 8.53 (s, 1H), 8.26 (dd, J=11, 6 Hz, 1H), 7.65 (dd, J=10, 5 Hz, 1H), 3.61 (m, 2H), 3.30 (in, 4H), 3.03 (m, 2H), 2.93 (dd, J=8, 8 Hz, 2H), 2.10 (m, 4H), 1.96 (m, 2H), 1.79 (m, 4H); MS (ES) m/z 572 (Mt++).

### Example 211

### 3-Amino-6-[2-difluoro-4-(piperidin-1-ylsulfonyl)phenyl]-N-[S-(3-pyrrolidin-1-ylpropyl)pyridin-3-yl]pyrazine-2-carboxamiide hydrochloride

Starting material: 2,5-difluoro-4-(piperidin-1-ylsulfonyl)phenylboronic acid, yield 15%: ¹H NMR (CD₃OD, 400 MHz) δ 9.41 (m, 1H), 8.75 (m, 2H), 8.53 (s, 1H), 8.25 (dd, J=11, 8Hz, 1H), 7.58 (dd, J=10, 6 Hz, 1H), 3.60 (m, 2H), 3.37 (t, J=7 Hz, 2H) 3.10 (m, 4H), 3.01 (m, 2H), 2.93 (m, 2H) 2.12 (m, 2H), 2.06 (m, 2H), 1.54 (m, 4H), 1.43 (m, 2H), −20 1.06 (m, 2H); MS (ES) m/z 586 (M⁺+1).

### Example 212

### 3-Amino-6-[4-(piperidin-1-ylsulfonyl)phenyl]-N-[5-(3-pyrrolidin-1-ylpropyl)pyridin-3-yl]pyrazine-2-carboxamide hydrochloride

Starting materials: 4(piperidin-1-ylsulfonyl)phenylboronic acid. Purification by reversed phase chromatography (C18, water/acetonitrile gradient) gave after precipitation the title compound, 26%.yield: ¹H NMR (CD₃OD, 400 MHz) δ 9.42 (s, 1H), 8.80 (s, 2H), 8.50 (s, 1 H),8.29 (d, J=8 Hz, 2H), 7.74 (d, J=8 Hz, 2H), 3.61 (m, 2H), 3.23 (m, 2H), 3.02 (m, 2H), 2.89 (m, 6H), 2.14 (m, 2H), 2.06 (m, 21H), 1.95 (m, 2H), 1.52 (m, 4H), 1.32(m, 2H); MS (ES) m/z 550 (M++i).

### Example 213

### 3-Amino-N-[5-(3-pyrrolidin-1-ylpropyl)pyridin-3-yl]-6-(4-(pyrrolidin-1-ylsulfonyl)phenyl]pyrazine-2-carboxamide hydrochloride

Starting materials: 4-(pyrrolidin-1-ylsulfonyl)phenylboronic acid, yield 23%: ¹H NMR (CD₃OD, 400 MHz) δ 9.43 (s, 1H), 8.84 (s, 1H), 8.78 (s, 1H), 8.50 (s, 1H), 8.34 (d, J=8 Hz, 2H), 7.87 (d,J 8 Hz, 2H), 3.67 (m, 2H), 3.29 (m, 2H), 3.21 (m, 4H), 3.08 (m, 2H), 2.97 (dd, J=8, 8, 0.2H), 2.15 (m, 4H), 2.00 (m, 2H), 1.70 (m, 4H); MS (ES) m,z 536 (M⁺+1).

### Example 214

### 3-Amino-N.[4-(2-pyrrolidin-1-ylethyl)pyridin-3-yl]-6-[4-(pyrrolidin-1-ylsulfonyl)phenyl]pyrazine-2-carboxamide hydrochloride

3-Amino-6-[4-(pyrrolidin-1-ylsulfonyl)phenyl]pyrazine-2-carboxylic acid (66 mg, 0.19-mmol), 4-(2-pyrrolidin-1-ylethyl)pyridin-3-amine (30 mg, 0.16 mmol), 2-(1H-benzotriazol-1-yl)-1, 1,3,3-tetramethyluroniumtetrafluoroborate (91 mg, 0.28 mmol), 1-hydroxybenzotriazole hydrate (35 mg, 0.26 mmol) and N,N-diisopropylethylamine (0.1 mL, 0.57 mmol) were suspended under inert gas atmosphere in acetonitrile (8 mL) and stirred at room temperature for 12 h. The solvent was removed in vacuo and the residue was separated between methylene chloride and saturated aqueous sodium hydrogen carbonate solution. The organic layer was dried over sodium sulfate. Filtration and removal of the solvent in vacuo gave a crude product, which was purified by chromatography on silica gel using a gradient ethyl acetate/heptane, (4:1); to ethyl acetate/methanol, (1:2), as the eluent. The product was dissolved in 3 in L of a methylene chloride/methanol mixture (v/v=3:1) and of hydrochloride acid in diethyl ether (51 M) was added. The precipitate was washed with diethyl ether and dried in vacuo to give 15 mg (15% yield) of the title compound: ¹H NMR (D₂O, 400 MHz) δ 9.12 (s, 1H), 8.80 (s, 1H), 8.62 (d, J=6Hz, 1H), 8.20 (d, J=9 Hz, 2H), 7.95 (m, 3H), 3.54 (m, 4H), 3.38 (m, 2H), 3.26 (m, 4H), 2.97 (m, 2-H), 1.87 (m, 2H), 1.81 (m, 2H), 1.70 (m, 4H); MS (ES) mnz 522 (M⁺+1).

The following Examples 215-216, were synthesized as described for Example 214: Example 215

### 3-Amino-N-[4-(3-pyrrolidin-1-ylpropyl)pyridin-3-yl]-6-[4-(pyrrolidin-1-ylsulfonyl)phenyl]pyrazine-2-carboxain-de hydrochloiode

Starting material: 4-(3-pyrrolidin-1-ylpropyl)pyridin-3-amine, yield 1:1 M D₂O, 5400 MHz) δ 9.25 (s, 1. H), 8.82 (s, 1H), 8.60 (d, J=6 Hz, 1H),8.20 (d, J=9 Hz, 2H)) 8.00 (d, J=6 Hz, 1H), 7.96 (d, J=9 Hz, 2H), 3.46 (m, 2H), 3.27 (m, 4H), 3.19 (t, J=8 Hz, 2H), 3.07 (t, J=8 Hz, 2H), 2.79 (m, 2H), 2.11 (m, 2H), 1.89 (m, 2H), 1.78 (m, 2H), 1.71 (m, 4H); MS (ES) m/z 536 (M++I).

### Example 216 ylsulfonyl)phenyl]pyrazine-2carboxamide hydrochloride

Starting material: 4-(pyrrolidin-1-ylmethyl)pyridin-3-amine. Purification by chromatography on silica gel using a gradients ethyl acetate/heptane, (4:1), to ethyl acetate/methanol, (2:1), as the eluent, followed by formation of the hydrochloric salt in 3 of a methylene chloride/methanol mixture (v/v 3: 1) by the addition of hydrochloride acid in diethyl ether (5 mL, 1 M) gave, after washing with diethyl ether and drying, 50 mg (34% yield) of the tide compound: ¹H NMR (D₂O, 400 MHz) δ 8.78 (m, 1H), 8.68 (m, 1H), 8.66 (s, 1H), 8.04 (d, J=9 Hz, 2H), 7.91 (d J=5 Hz, 1H), 7.71 (d, J=9 Hz, 2H), 4.56 (s, 2H), 3.3 8 br s, 4H), 3,11 (t, J66 Hz, 4H), 2.01 (ni, 4H), 1.61 (m, 4H); MS (ES) m/z 508 M+)

### Example 217′

### ylsulfonyl)phenyl]pyrazine-2-carboxamde hydrochloride

4(Pyrrolidin-1-ylsulfonyl)phenylborornc acid (174 mg, 0.68 mmol), 3-amino-6-bromo-N-(4-[(dimethylamino)methylpyridin-3-yl)pyrazine-2-carboxamide (220 mg, 0.62 mmol), sodium carbonate (181 mg in 0.9 Ti water, 1.71 mmol), Pd(dppf)Cl₂×CH₂Cl₂ (40 mg, 0.05 mmol) were dissolved under inert gas atmosphere in tetrahydrofuran (10 Ti) and the reaction mi xture was heated to 50° C. and stirred for 2 h. Lithium chloride (100 mg, 2.3 mmol).and Pd(PPh₃)₄ (20 mg, 0.01 mmol) and Pd(dppf)Cl₂xCH Cl₂ (30 nmg, 0.04 mmol) were added and stirring at 50° C. was continued for 10 h. Saturated aqueous sodium chloride solution (5 mL) and ethyl acetate (15 mL) and tetrahydrofuran (20 mL) were added. The layers were separated and the organic layer was dried over magnesium sulfate. Filtration and removal of solvent in vacuo gave a residue, which was purified by column chromatography on silica gel using a gradient of ethyl acetate/heptane, (1:1), to ethyl acetate/methanol, (11), as eluent. The product was dissolved in 3 mL of a methylene chloride/methanol mixture (v/v=3:1) and of hydrochloride acid in diethyl ether (5 mL, 1 M) was added. The precipitate was washed with diethyl ether and dried in vacuo'to give 35-mg (10% yield) of the title compound: ¹H NMR (D₂O, 400 MHz) δ 9.10 (m, 1H), 8.85 (m, 1H), 8.66 (m, 1H), 8.24 (m, 1H), 8.00 (m, 2H), 7.72 (m, 2H, 4.66 (s, 2H), 3.14 (s, 4 H), 2.92 (m, 6 H), 1.61 (m, 4H); ° C. NMR (D₂O, 100 MHz) δ 166.7, 154.2, 145.4, 144.1, 142.5, 141.5, 140.0, 138.4, 136.1, 134.5, 128.9, 128.3, 126.5, 124.4, 55.7, 48.7, 43.8, 25,1; MS (ES) m/z 482 (M⁺+1).

### Example 218

### 3-Amino-N-{4-[(dimethylaminoymethyl]pyridin-3-yl}-6-14-(piperidin-1-ylsulfonyl)phenyl]pyrazine-2-carboxamide hydrochloride

The title compound was prepared as described for Example 217 using 4(piperidin-1-ylsulfonyl)phenylborbnic acid, yield 14%: ¹H NMR (D₂O, 400 5 Hz) δ 9.03 (s, 1H), 8.80-(m, J=6 Hz),8.69 (s, 1H), 8.17 (m, 1H), 8.06 (d,J 7 Hz, 2H), 7.69(d, J=7 Hz, 2H), 4.62 (s, 2H), 2.91 (s, 6H), 2.88. (s, 4H), 1.50 (m, 4H), 1.32, (m, 2H); MS (ES) m/z 496

The following Examples, 219-225, were synthesized as described for Example 240:

### Example 219

### 3-Amino-6-[3-methyl-4-[(4-methylpiperaiin-1-yl)sulfonyl]phenyl}-N-pyridin-3-ylpyrazine-2-carboxamide hydrochloride

Starting material: 3-amino-6-{3-ethyl-4-[(4-methylpiperazin-1-yl)sulfonyl]phenyl}-N-pyridin-3-ylpyrazine-2-carboxamide, yield 99%: ¹H NMR (D₂O)O 9.34 (d, J=2 Hz, 1H), 8.60 (s, 1 H), 8.56(d, J=0.6 Hz, 1H), 8.51 (m, 1H), 8.01 (dd, J=9; 6 Hz, 1H), 7.91 (s, 1H), 7.84 (dd, J 8, 2 Hz, 1H), 7.76 (d, J=8 Hz, 1H), 3.87 (m, 2H), 3.59 (m, 2H), 3.16 (m, 4H), 2.91 (s, 3H), 2.89 (q, J=8 Hz, 2H); 1.26 (t, J=8 Hz, 3Hj

### Example 220

### 3-Amino-6-[4-[(4-methylpiperazin-1-yl)sulfonyl]3-(trifluoromethoxy)phenyl]-N-pyridin-3-ylpyrazine-2-carboxamide hydrochloride

Starting material: 3-amino-6-[4-[(4-methylpiperazin-1-yl)sulfbnyl]-3-(trifluoromethoxy)phenyl]-N-pyridin-3-ylpyrazine-2-carboxamide: yield 96%: ¹H NMR (D₂O) δ 9.34 (s, 1H), 8.66 (s, 1H), 8.56 (d, J=5 Hz, 1H), 8.44 (d, J=9 Hz, 1H), 7.98 (m, 3H), 7.87 (d, J=9 Hz, 1H), 3.98 (m, 2H), 3.60 (m, 2H), 3.19 (m, 4H), 2.91 (s, 3H); “C NMR (D₂O) δ 165.0, 154.6, 146.6, 145.8, 142.7, 137.9, 137.4, 136.0,135.9, 133.3, 132.6, 127.9, 127.7, 124.2, 123.6, 117.5, 53.3, 43.29, 43.1; MS.(TSP) m/z 538 (M?+1)

### Example 221

### 3-Amino-6-[4-{[(2-aminoethyl)aminosulfonyl}-3-(trifluoromethoxy)phenyl]-N-pyridin-3-ylpyrazine-2-carboxamide hydrochloride

Starting material: 3-amino-6-(4-{[(2-aminoethyl)amino]sulfonyl}-3-(trifluoromethoxy)phenyl]-N-pyridin-3-ylpyrazine-2-carboxamide, yield 95%: ¹H NMR (D20, 400 MHz 5 9.33 (d, J=2 Hz, 1H), 8.67 (s, 1H), 8.55 (d, J=5 Hz, 1H), 8.44 (m, 1H), 8.00 (m, 2H), 7.96 (m, 1H), 7.91 (m, 1H), 3.25 (m, 2H), 3.15 (m, 2H); MS (TSP) m/z 498 (M*+1).

### Example 222

### 4-Amino-4′-[(4-methylpiperazin-1-yl)sulfonyl]-N-pyridin-3-yl1,1′-biphenyl-3-carboxamride hydrochloride

Starting material: 4-amino-4′-[(4-methylpiperazin-1-yl)sulfonyl]-N-pyridin-3-yl-1,1′-biphenyl-3-carboxamide, yield 62%: ¹H NMR (D₂O, 400 MHz) δ 9.26 (s, 1H), 8.50 (m, 2H), 7.96 (m, 1H), 7.84 (m, 1H), 7.78 (d, J=8 Hz, 2H), 7.71 (d, J=9 Hz, 2H), 7.64 (m, 1H), 6.98 (d, J=9 Hz, 1H), 3.86 (d, J=14 Hz, 2H), 3.55 (d, J=13 Hz, 2H), 3.18 (m, 2H), 2.83 (s, 3H), 2.75 (m, 2H); ¹³C NMR (D20) δ 145.0, 144.95, 138.5, 137.3, 136.7, 133.1, 132.6, 131.9, 130.1, 128.6, 127.9, 127.8, 127.4, 120.7, 118.0, 52.9, 43.5, 43.1.

### Example 223

### 2-Amino-5-14-[(4-methylpiperazin-1-yl)sulfonyl]phenyl}-N-[4-(pyrrolidin-1-ylmethyl)pyridin-3-yl]nicotinamide hydrochloride

Starting material: 2-amino-5-(4-[(4-methylpiperazin-1-yl)sulfonyl]phenyl)-N-[4-(pyrrolidin-1-ylmethyl)pyridin-3-yl)nicotinamide, yield 85%: ¹H NMR (D₂O, 400 MHz) 8-9.04 (d, J=2 Hz, 1H), 8.90 (s, 1H), 8.78 (d, J=6 Hz, 1H), 8.51 (d, J=2 Hz, 1H); 8.04 (d, J=6 Hz, 1H), 7.99 (m, 4H), 4.68 (s, 2H), 3.98 (m 2H), 3.61 (m, 2H), 3.26 (br m, 6H), 2.90 (s, 0.3H), 2.89 (br m, 2H), 2.10 (m, 4H); MS (ES) m/z 536 (MW+1).

### Example 224

### 3-Amino-N-pyridin-3-yl-6-[4-(pyrrolidin-1-ylsulfonyl)phenyl]pyrazine-2-carboxamide hydrochloride

Starting material: 3-amino-N-pyridin-3-yl-6-[4-(pyrrolidin-1-ylsulfonyl)phenyl)pyrazine-2-carboxamide, yield 97%: ¹³C NMR (CD₃OD, 100 MHz) δ 166.72, 156.06, 147.03, 141.26, 139.74, 139.30, 138.16, 137.43, 137.14, 134.08, 129.17, 128.20, 127.48, 127.48, 124.58, 26.22; MS (ES) nz/z 425 (M⁺+1).

### Example 225.

### 3-Amino-6-[4-(piperidin-1-ylsulfonyl)phenyl]-N-pyridin-3-ylpyrazine-2-carboxamide hydrochloride

Starting material: 3-amino-6-[4-(piperidin-1-ylsulfonyl)phenyl]-N-pyridin-3-ylpyrazine-2-carboxamide, yield 95%: ¹H NMR (CD₃OD, 400 MHz) δ 9.54 (m, 1H), 8.84 (m, 2H), 8.56 (d, J=9 Hz, 1H), 8.31 (dJ=8 Hz, 2H), 8.9 (d, J=8, 6 Hz, 1H), 7,82 (dJ=8 Hz, 2H), 2.97 (t, J=6 Hz, 4H), 1.59 (m, 4H), 1.40 (m, 2H);MS (ES) m/z 439 (M⁺+1).

### Example 226

### 3-Amino-6-[4-(piperazin-1-ylsulfonyl)phenyl]-N-pyridin-3-ylpyrazine-2-carboxamide hydrochloride

A solution of tert-butyl 4[(4-(5-amino-6-[(pyridin-3-ylamino)carbonyl)pyrazin-2-yl)phenyl)sulfonyl]piperazine-1-carboxylate (0.3 g, 0.56 mmol) in methanolimethylen chloride (10:2 mL) was heated at 60° C. for 6 h. The solvent was evaporated. The resulting residue was dissolved in methanol/water, (2:1), filtered, and the solvent evaporated to give 0,28 g (98% yield) of the title compound: ¹H NMR (D20, 400 MHz) δ 8.31 (d, J=2 Hz, 1H), 8.58 (s, 1H) 8.53 (m, 1H), 8.50 (m, 1H), 8.05 (d, J=8 Hz, 2H), 7.97 (dd, J=9, 6 Hz, 1H), 7.67 (d, J=8 Hz, 2H), 3.26 (br s, 8H); ³C NMR (D₂O, 100 MHz) δ 165.1, 154.1, 145.5, 140.3, 137.6, 137.4, 137.1, 137.0, 133.5, 132.7, 128.5, 128.0, 126.4, 123.9, 43.1, 43.1; MS (ES) m/z 440.20 (M⁺+1).

### Example 227

### 3-Amino-6-[4-{[(2-aminoethyl)amino]sulfonyl}-3-(trifluoromethoxy)phenyl]N-pyridin-3-ylpyrazine-2-carboxamide

HCl in diethyl ether (1.0 M, 25-mmol) was added to a solution of tert-butyl 2-([(4-(5-amino-6-[(pyridin-3-ylamino)carbonyl]pyrazin-2-yl}phenyl)sulfonyl]-(tert-butoxycarbonyl)amino)ethylcarbamate in methanol (40 mL). The resulting mixture was stirred under reflux for 38 h. The solvent was evaporated and a mixture of saturated aqueous sodium carbonate and methylene chloride was added. The organic phase was washed with water, dried over-MgSO₄, and the solvent was evaporated. Purification by column chromatography using methylene chloride/methanol, (7:3), gave 0.26 g (62% yield) of the title compound as a brown solid: ¹H NMR (DMSO-d₆, 400 MHz) δ 10.67 (s,
H), 9.07 (s, 1H), 8.96 (d, J=2z,1H), 8.43 (dd, J=8, 1 Hz, 1H), 8.37 (m, 1H), 8.34 (m, 1 H), 8.2Z (m, 1H), 7.99 (d, J=8 Hz, 1H), 7.93 (br s, 2 H), 7.45 (dd, J=8, 5 Hz, I 20H), 2.87 (t, J=7 Hz, 2H), 2.55 (t, J=7 Hz, 2H); MS(ES) m/z498+(M⁺+1)

### Example 228

### 3-Amino-6-[4-(2piperazin-1-ylethoxy)phenyl]-N pyridin-3-ylpyrazine-carboxamide hydrochloride

HCl (4.2mL, 1.0 M in diethyl ether) was added dropwise to a cooled (0° C.) solution of tert-butyl 4-[2-(4-(5-amino-6-[(pyridin-3-ylamino)carbonyl]pyrazin-2-yl)phenoxy)ethyl]piperazine-1-carboxylate (0.300 g, 0.58 mmol) in methanol (35 mL). The solution was stirred at room temperature for 92 h. The solvent was evaporated and resulting solid was dissolved in refluxing methanol (160 mL). HCl (4.0 mL 0.7 M in diethyl ether) was added and the resulting mixture was heated at reflux for 2 h. The solvent was evaporated and the residue was dried under vacuum to give 0.25 g (88% yield) of the title compound: ¹HNMR (DMSO-d₆, 300 MHz) δ 11.09 (s, 1H), 10;00 (br s, 2H), 9.41 (d, J=2 Hz, 1H), 8.95 (s, 1H), 8.93 (m,I 1H), 8.68 (d, J=5 Hz, 1H), 8.23 (d, =9 Hz, 2H), 8.06 (dd, J 9, 6 Hz, 1H), 0.12 (d J 9 Hz, 2H), 4.51 (m, 2H), 3.57 (m, 10H); MS (ES) m/z 420 (M⁺+1).

### Example 229

### 3-Amino-6-[2,5-difluoro-4-(2-piperazin-1-ylethoxy)phenyl]-N-pyridin-3-ylpyrazine-2-carboxamide hydrochloride

The title compound was prepared as described for Example 228 using tert-butyl 4-[2-(4-0.15-amino-6-[(pyridin-3-ylamino)carbonyllpyrazin-2-yl)-2,5-difluorophenoxy)ethyl]piperazine-1-carboxylate. Heating at 60° C. for 1 h was enough to complete the reaction, yield 91%: ¹H NMR (DMSO-d₆, 300 MHz) δ 10.99 (s, 0.1H), 9.79 (br s, 2H), 9.29 (s, 1H), 8.74 (s, 1H), 8.73 (m, 1H), 8.62 (d, J.=5 Hz, 1H), 8.33 (dd, J=12, 7 Hz, 1H), 7.92 (dd, J 8, 5 Hz, 1H), 7.80 (br s, 1H), 7.40 (dd, J=13, 7 Hz, 1H), −4.61 (m, 2H), 3.66 (m, 2H), 3.58 (m, 4H), 3.49 (m, 4H).

### Example 230

### 3-Amino-6-[5-(piperazin-1-ylcarbonyl)-2-furyl]-N-pyridin-3-ylpyrazine-2-carboxamide hydrochloride

tert-Butyl 4-(5-(5-amino-6-[(pyridin-3-ylamino)carbony1)pyrazin-2-yl)-2-furoyl)piperazine-1-carboxylate (97 mg, 0.2 mmol) was dissolved in methanol (2 mL) and methylene chloride (2 nL). Hydrochloric acid (1 mL, 1 M in diethyl ether) was added and the reaction mixture was heated for 3 h at reflux. The solvent was removed in vacuo'and the residue was washed with meth ene chloride/methanol 3 mL, 1) to give 40 mg (43% yield) of the title compound as a yellow solid: ¹H NMR (D₂O, 400 MHz) δ 9.35 (m, 1H), 8.63 (ddd, J=9, 2, 1 Hz, 1H), 8.53 (m, 1H), 8.45 (s, 1H), 8.01 (m, 1H), 7.08 (d, J=4 Hz, 1H), 7.00 (d, J=4 Hz, 1H), 4.05 (br s, 4H), 3.38 (m, 4H), ¹³C NMR (D2O, 100 MHz) δ 165.2, 160.3, 153.9, 152.8, 144.7, 144.6, 137.8, 137.2, 136.8, 133.0, 131.7, 127.9, 123.7, 120.0, 109.2, 43.3; MS (ES) m/z 394 (M⁺+1).

### Example 231

### 3-Amino-N-{5-[3-(dimethylamino)-ropyl]pyridin-3-y l}6-[4o(piperidin-1-ylsulfonyl)phenyl]pyrazine-2-carboxamide hydrochloride HCl in diethyl ether (1 M, 5 mL) was added dropwise to a cooled (0 IC) solution of 3-amino-N-{5-[3-(dimethylamino)propyl]pyridin-3-yl)-6-[4-(piperidin-1-ylsulfonyl)phenyl]pyrazine-2-carboxamide (0.175 g, 0.33 mmol) in methylene chloride (Io ML). The mixture was stirred for 30 min at 0° C. The precipitate was filtered and washed with diethyl ether and dried. Purification by reversed phase chromatography (column: XTerra C8 19x300 mm) using a water/acetonitrile gradient gave 53 mg of the starting compound. The salt formation described above was repeated to give 41 mg, (85% yield) of the title compound: MS (ES) m/z 524 (M⁺+1).

### Example 232

### 4-{5-Amino-6-[(pyridin-3-ylamino)carbonyl]pyrazin-2-yl}benzoic acid

Pd(PPh₃)₄ (1.05 g, 0.91 mmol) was added to a to a solution of 3-amino-6-bromo-N-pyridin-3-ylpyrazine-2-carboxamide (2.0 g, 6.8 mmol), 4-carboxyphenylboronic acid (1.12 g, 6.7mmol), and sodium carbonate (2.88 g, 27.2 mmol) in tetrahydrofuran/water, (1:1, 240 mL), and the resulting mixture was heated at 75° C. for 16 days. The solvent was evaporated and the residue dissolved in water. The aqueous phase was extracted with ethyl acetate and then neutralized (pH 7) using HCl (10% aq). The formed crystals were filtered off and dried in vacuo to give 1.7 g (77% yield) of the title compound: MS (ES) m/z 336 (M⁺+1).

### Example 233

### 3-Amino-64-3(dimethylamino-3sulfonyl]phenyl}-N-pyridin-3-ylpyrazine-2-carboxamide

In a round bottom flask fitted with a condenser, a mixture3-amino-6-bromo-N-pyndin-3-ylpyrazine-2-carboxamide (23 mg, 78 μmol), N″-dimethyl-4-(4,4,5,5-tetramethyl-1,3,2-dioxaborolan-2-yl) benzenesulfonamide (24 mg, 78 mmol) and Pd(dppf)Cl₂×CH₂Cl₂ (3.2 mg, 3.9 μmol) in toluene (2 mL), ethanol (0.2 mL) and Na₂CO₃ solution (2 M, 0.2 mL) was stirred at 80° C. over night. Silica gel (0.5 g) was added to the reaction mixture and the mixture was concentrated to dryness. The residue was purified on a silica gel column using heptane/ethyl acetate, (1:1), as the eluent to give 30 mg (96% yield) of the title compound: ¹H NMR (DMSO-d₆, 400 MHz) δ 10.62 (s, 1H), 9.05 (s, 1H), 8.98 (d, J=2 Hz, 1H), 8.52-8.50 (m, 2H), 8.37 (dd, J=5, 1 Hz, 1H), 8.22 (ddd, J=8, 2 and 2 Hz, 1H), 7.85 (br s, 2H), 7.83-7.81 (m, 2H), 7.45 (dd, J=8, 5 Hz, 1H), 2.65 (s, 6H); MS (ES)/Z 399

### Example 234

### 3-Amino-6-{3-[(dimethylamino)sulfonyl]phenlyl}-N-pyridin-3-ylpyrazine-2-carboxamide

The compound was prepared as described for Example 233 using N,N-dimethy1-3-(4,4,5,5-tetramethyl-1,3,2-dioxaborolan-2-yl)benzenesulfonamide: yield 24%; mp 234.5-238.0° C.; ¹H NMR (DMSO-d₆, 400 MHz) δ 10.65 (s, 1H), 9.03,(s, 1H), 8.96 (d, J=2 Hz, 1H), 8.64-8.61 (m, 1H), 8.37-8.36 (m, 2H), 8.21 (ddd, J=8, 2 and 3 Hz, 1H), 7.80 (br s, 2H), 7.78-7.75 (m, 2H), 7.44 (dd, J=8, 5 Hz, 1H), 2.68 (s, 6H); MS (ES) m/z 399 (M⁺+1).

### Example 235

### 3-Amino-6-{2-[(dimethylamino)sulfonyl]phenyl}-N-pyridin-3-ylpyrazine-2-carboxamide

Tbe compound was prepared as described for Example 233 using N,N-dimethyl-2-(4,4,5,5-tetramethyl-1,3,2-dioxaborolan-2-yl)benzenesulfonamide: yield: 60%; mp 221.5-223.0° C.; ¹H NMR (DMSO-d₆, 400 MHz) δ 10.56 (s, 1H), 8.90 (d, J=2 Hz, 1H), 8.52 (s, 1H), 8.32 (dd, J=5, 1 Hz, 1H), 8.19 (ddd, J=8, 2 and 2 Hz, 1H)I 7.87 (d, =8 Hz, 1H), 7.79-7.78 (m, 2H), 7.75 (br s, 2H), 7.70 (dq, J=12 and 4 Hz, 1-H), 7.41 (dd, J 8,5 Hz, 1H), 2.75 (s, 6H); ¹³C NMR (MSO-d6, 100 MHz) δ 164.90, 154.14, 141.59, 144.90, 141.74, 139.05, 137.08, 136.46, 134.82, 132.73, 132.69, 129.16, 128.39, 127.08, 123.74, 122.26, 37.13; MS (ES) m/z 399 (M⁺+1).

### Example 236

### 3-Amino-6-[4-(aminosulfonyl)phenyl]-N-pyridin-3-ylpyrazine-2-carboxamide

The compound was prepared as described for Example 233 using 4-(4,4,5,5-tetramethyl-0.1,3,2-dioxaborolan-2-yl)benzenesulfonamide: yield 40%; ¹H NMR (DMSO-d₆, 400 MHz) 3 10.61 (br s, 1H), 9.03 (s, 1H), 8.98 (d, J=2 Hz, 1H), 8.44 (d, J=9 Hz, 2H), 8.37 (dd, J=5, 1Hz, 1H), 8.21 (ddd, J=8,2 and 2 Hz, 1H), 7.90 (d, J=8 Hz, 2H), 7.82 (brs, 2H), 7.44 (dd, J=8, 5 Hz, 1H); ¹³C NMR (DMSO-d₆, 100 MHz) 3 165.06, 154.70, 145.52, 145.22, 143.46, 143.27, 138.86, 137.03, 134.63, 128.73, 126.06, 126.01, 123.85, 123.56; MS (ES),7/z 370.97 (M⁺+1).

### Example 237

### 2-Amino-5-{4-[(dimethylamino)sulfonyl]pfienyl}-N-pyridin-3-ylnicotinamide-

2-Amino-5-bromo-N-pyridin-3-ylnicotinamide (0.10 g, 0.34 mmol), N,N-dimethyl-4-(4,4,5,5-tetramethyl-1,3,2-dioxaborolan-2-yl)benzenesulfonamide (0.13 g, 0.41 mmol) and Pd(dppf)Cl₂XCH₂Cl₂ (12.4 mg, 17.2 lmol) were mixed in toluene/ethanol, (1: 1, 2 mL), and saturated Na₂CO₃ (aq) solution (6.20 mL). Nitrogen gas was bubbled through the reaction mixture for 5 min and the mixture was heated for 16 h. Silica gel was added and the solvent was evaporated. The residue was purified on a silica gel column, using a gradient heptane 100% to ethyl acetate 100% as the eluent, to give 95 nmg (79% yield). Additional purification using reversed phase chromatography (C18, water/acetonitrile gradient) gave 36 mg (26% yield) of the title compound as a solid: ¹H NMR (DMSO-d₆, 400 MHz) δ 2.70 (s, 6H), 7.87 (d, J=9 Hz, 2H), 7.93 (dd, 6 Hz, 1H), 8.13 (d, J=9 Hz, 2H), 8.62(d, J=6 Hz I H),8.67 (d, J=9 Hz, 1H), 8.71 (d, J=2 Hz, 1H), 8.87 (s, 1H), 9.28 (s, 1H), 11.60 (s, 1H); MS (ES) m/z 398 (M⁺+1).

### Example 238

### 3-Amino-6-(4-(C(3-morpholin-4-ylpropyl)amino]sulfonyl)phenyl)-N-pyridin-3-ylpyrazine-2-carboxamide

The title compound was prepared as described for Example 233 using 4-([(3-morpholin-4-ylpropyl)amino]sulfonyl}phenylboronic acid: yield 86%: mp 219-227° C. (decomnp.); 1H NM (DMSO-d₆, 400 MHz) δ 10.61 (s, 1H), 9.03 (s, 1H), 8.99 (m, 1H), 8.47 (d, =8-Hz, 2H), 8.38 (d, J=4 Hz, 1H),8.22 (m, 1H), 7.86 (d, =J8 Hz, 4H), 7.69 (t, J=6 Hz, I H), 7.45 (dd,3J 8, S Hz, 1H), 3.50 (t, J=4 Hz, 4H), 2.80 (q, J=7 Hz. 2H), 2.22 (m, 6H), 1.52 (quint, J=7 Hz, 2H); ¹³C NMR.(DMSO-d₆, 100 MHz) 3 165.0, 154.7, 145.5, 145.2, 143.2, 139.6, 139.3, 136.8, 134.6, 128.6, 126.9, 126.2, 123.8, 123,5, 66.2, 55.3, 53.2, 40.8, 25.9; MS (ES) m/z 498 (M+++1).

### Example 239

### 3-Amino-6-(4-[(4-methylpiperazin-1-yl)sulfonyl]phenyl)-N-pyridin-3-ylpyrazine-2-carboxamide

The title compound was prepared as described for Example 233 using 4-[(4-methylpiperazin-1-yl)sulfonyl]phenylboronic acid: yield: 79%; mp decomposes 220-2296C; ¹H NMR (DMSO-d₆, 400 MHz) δ 9.85 (br s, 1H), 8.88 (br s, 1H), 8.75 (s, 1H), 8.45 (d, J=4 Hz, 1H), 5.30 (m, 1H), 8.07 (d, J=8 Hz, 2H), 7.88 (d, J=8 Hz, 2H), 7.37 (dd, J=8, 5 Hz, 1H), 3.37 (m, 4H), 2.92 (m, 4H), 2.56 (m, 3H); ¹³C NMR (DMSO-d6, 100 MHz) 56172.1, 162.6, 153.7, 153.6, 149.6, 146.2, 142.9, 142.0, 136.4, 135.0, 134.4, so 132.5, 131.7, 61.5, 52.6; MS (TSP) m/z 454 (M⁺+1).

### Example 240

### 3-Amino-6-{4-[(4-methylpiperazin-1-yl)sulfonyl]phenyl)-N-pyridin-3-yipyrazine-2-carboxamide hydrochloride

HCl in diethyl ether (1 M, 0.81 mL) was added to a solution of 3-amino-6-{4-[(4-methylpiperazin-1-yl)sulfonyl]phenyl}-N-pyridin-3-ylpyrazine-2-carboxamide (0.096 g, 0.21 mmol) in of methylene chloride/methanol, (0.95:0.05, 8 mL). The yellow precipitate was filtered off, washed with diethyl ether and dried under vacuo to give the title compound as a yellow solid: mp 217-223° C. (decomp.).

Pharmaceutical Formulations

According to one aspect of the present invention there is provided a pharmaceutical formulation comprising a compound of formula I, as a free base or a pharmaceutically acceptable salt thereof, for use in the prevention and/or treatment of conditions associated with glycogen synthase kinase-3.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream or for rectal administration as a suppository.

In general the above compositions may be prepared in a conventional manner using conventional excipients, pharmaceutical diluents or inert carriers. Suitable daily doses of the compounds of formula I in the treatment of a mammal, including man are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

The following illustrate representative pharmaceutical dosage forms containing a compound of formula I, as a free base or a pharmaceutically acceptable salt thereof, (hereafter compound X), for therapeutic or preventive use in mammals:

| (a): Tablet | Mg/tablet |
|---|---|
| Compound X | 100 |
| Lactose | 182.75 |
| Croscarmellose sodium | 12.0 |
| Maize starch paste (5% w/v paste) | 2.25 |
| Magnesium stearate | 3.0 |
| (b): Capsule | Mg/capsule |
| Compound X | 10 |
| Lactose | 488.5 |
| Magnesium stearate | 1.5 |
| (c): Injection | (50 mg/ml) |
| Compound X | 5.0% w/v |
| 1M Sodium hydroxide solution | 15.0% v/v |
| 0.1M Hydrochloric acid | (to adjust pH to 7.6) |
| Polyethylene glycol 400 | 4.5% w/v |
| Water for injection | up to 100% |

The above formulations may be obtained by conventional procedures well known in the pharmaceutical art.

Medical Use

Surprisingly, it has been found that the compounds defined in the present invention, as a free base or a pharmaceutically acceptable salt thereof, are well suited for inhibiting glycogen-synthase kinase-3 (GSK3). Accordingly, the compounds of the present invention are expected to be useful in the prevention and/or treatment of conditions associated with glycogen synthase kinase-3 activity, i.e. the compounds may be used to produce an inhibitory effect of OSK3 in mammals, including man in need of such prevention and/or treatment.

GSK3; is highly expressed in the central and peripheral nervous system and in other tissues. Thus, it is expected that a the compounds of the invention are well suited for the prevention and/or treatment of conditions associated with glycogen synthase kinase-3 in the central and peripheral nervous system. In particular, such compounds of the invention are expected to be suitable for prevention and/or treatment of conditions associated with especially, dementia, Alzheimer's Disease, Parkinson's Disease, Frontotemporal dementia Parkinson's Type, Parkinson dementia complex of Gaum, HIV dementia, diseases with associated neurofibrillar tangle pathologies, amyotrophic lateral sclerosis, corticobasal degeneration, dementia pugilistica, Down syndrome, Huntington's Disease, ppstencephelatic parkinsonism, progressive supranuclear palsy, Pick's Disease, Niemann-Pick's Disease, stroke, head trauma and other chronic neurodegenerative diseases, Bipolar Disorders, affective disorders, depression, schizophrenia, cognitive disorders, Type I and Type I diabetes and diabetic neuropathy, hair loss and contraceptive medication.

The dose required for the therapeutic or preventive treatment of a particular disease will necessarily be varied depending on the host treated, the route of administration and the severity of the illness being treated.

The present invention relates also to the use of a compound of formula I as defined hereinbefore, in the manufacture of a medicament for the prevention and/or treatment of conditions associated with GSK3.

In the context of the present specification, the term “therapy” includes treatment as well as prevention, unless there are specific indications to the contrary. The terms “therapeutic” and “therapeutically” should be construed accordingly.

The invention also provides a method of treatment and/or prevention of conditions associated with,GSK3, in a patient suffering from, or at risk of, said condition, which comprises administering to the patient an effective amount of a compound of formula I, as hereinbefore defined.

Non-Medical Use

In addition to their use in therapeutic medicine, the compounds of formula I as a free base or a pharmaceutically acceptable salt thereof, are also useful as pharmacological tools in the development and standardisation of in vitro and in vivo test systems for thei evaluation of the effects of inhibitors of GSK3 related activity in laboratory animals such as cat, dogs, rabbits, monkeys, rats and mice, as part of the search for new therapeutics agents.

Pharmacology

Determination OfATP competition in Scintillation Proximity GSK3,8Assay.

GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK30 (Dundee University, UK), 12 mM morpholinepropanesutlfonic acid (MOPS), pH 7.0, 0.3 mrM EDTA, 0.01% β-mercaptorethanol, 0.004% Brij 35 (a natural detergent), 0.5. % glycerol and 0.5 μg BSA/25 μl. The reaction was initiated by the addition of 0.04 μCi [γ-³³P]ATP (Amersham, UK) and unlabelled ATP at a final concentration of 1 MM and assay volume of 25 μl. After incubation for 20 minutes at room temperature, each reaction was terminated by the addition of 25 μl stop solution containing 5 mM EDTA, 50 μM ATP, 0.11% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The KF value of ATP for GSK30, used to calculate the inhibition constants (Ki) of the various compounds, was 20 μM.

The following abbreviations have been used:
MOPS Morpholinepropanesulfonic acid
EDTA Ethylenediaminetetraacetic acid
BSA Bovin Serum Albumin
ATP Adenosine Triphophatase
SPA Scintillation Proximity Assay
GSK3 Glycogen Synthase Kinase 3.
Pd(dppf)C12 [1.1′-Bis(diphenylphosphino)ferrocene]dichloropalladium(II)
Ni(dppe)C12 [1.1′-Bis(diphenylphosphino)ethane]dichloronickel(II)
   Results

Typical Kᵢ values for the compounds of the present invention are in the range of about 0.25 0.001 to about 10,000 nM, preferably about 0.001 to about 1000 nM, particularly preferred about 0.001 nM to about 300 nM.

1. A compound having the formula I 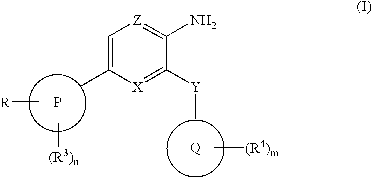
as a free base or a pharmaceutically acceptable salt thereof,
wherein:
Z is CH or N;
Y is CONR⁵, NR⁵CO, SO₂NR⁵, NR⁵SO₂, CH₂NR⁵, NR⁵CH₂, NR⁵CONR⁵, C₁₋₆alkylene, CH₂CO, COCH₂, CH═CH, OCH₂ or CH₂O;
X is CH or N;
P is phenyl or a 5- or 6-membered heteroaromatic ring containing one or more heteroatoms selected from N, O and S and said phenyl ring or 5- or 6-membered heteroaromatic ring is optionally fused with a 5- or 6-membered saturated, partially saturated or unsaturated ring containing one or more atoms selected from C, N, O and S;
Q is phenyl or a 5- or 6-membered heteroaromatic ring containing one or more heteroatoms selected from N, O and S of which at least one atom is selected from nitrogen;
R is CHO, fluoromethoxy, difluoromethoxy, trifluoromethoxy, C₀₋₆alkyl(SO₂)NR¹R², OC₀₋₆alkyl(SO₂)NR¹R¹, OC₁₋₆alkyl(SO)NR¹RC₁₋₆alkyl (SO)NR¹R¹², C₀₋₆alkylNR¹(SO)R², OC₁₋₆alkylNR¹(SO)R², C₀₋₆alkylNR¹(SO₂) NR¹R¹, OC₁₋₆alkylNR¹(SO₂)RC₀₋₆alkyl (SO₂) C₁₋₆alkylNR¹²R¹², OC₀₋₆alkyl (SO₂) C₁₋₆alkylNR¹R¹C₀₋₆alkyl (SO) C₁₋₆alkylNR¹R², OC₁₋₆alkyl(SO)C₁₋₆alkylNR¹R², C₀₋₆alkylSC₁₋₆alkylNR¹R², OC₁₋₆alkylSC₁₋₆alkylNR¹R², OC₁₋₆alkylOC₁₋₆alkyl, C₁₋₆alkylOC₁₋₆alkylNR¹R² OC₁₋₆alkylOC₁₋₆alkylNR¹R², C₀₋₆alkylCONR¹R², OC₀₋₆alkylCONR¹R², OC₁₋₆alkylNR¹R², C₀₋₆alkylNR¹⁰ (CO)R¹¹, OC₁₋₆alkylNR¹(CO)R², C₀₋₆alkylNR¹¹(CO)R¹⁰, C₀₋₆alkylCOR¹¹, OC₁₋₆alkylCOR¹, C₀₋₆alkylNR¹⁰OR¹¹, C₀₋₆alkylO(CO)R¹¹, OC₁₋₆alkylO (CO)R¹, C₀₋₆alkylC(NR¹⁰)NR¹OR¹¹, C₀₋₆alkylC(NR¹¹)N(R¹⁰)₂, OC₀₋₆alkylC(NR¹)NR¹R², C₀₋₆alkylNR¹⁰ (CO)OR”, OC₁₋₆alkylNR¹(CO)OR¹, C₀₋₆alkylNR¹¹(CO)OR¹⁰, OC₁₋₆alkylCN, NR¹ORC₀₋₆alkyl (CO)OR⁸, OC₁₋₆alkyl (CO)OR¹, NR¹² (CO)NR¹R¹², NR¹(CO) (CO)RNR¹(CO) (CO)NR¹¹R¹² or SO₃R^{1;}
R¹ and R² are independently selected from hydrogen, C₁₋₆alkyl, C₂₋₆alkenyl, C₂₋₆alkynyl, C₀₋₆alkylC₃₋₆cycloalkyl, (CO)OR⁸, C₀₋₆alkylheterocycloalkyl, C₁₋₆alkylNR⁶R⁷, C₀₋₆alkylaryl and C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylheterocycloalkyl, C₀₋₆alkylaryl, and C₀₋₆alkylheteroaryl may be substituted by one or more A;
R¹ and R² may together form a substituted 5-, 6- or 7-membered heterocyclic ring containing one or more heteroatoms selected from N, O r and S, which heterocyclic ring is optionally substituted by A;
R³ and R⁴ are independently selected from halo, nitro, CHO, C₀₋₆alkylCN, OC₁₋₆alkylCN, C₀₋₆alkylOR⁶, OC₁₋₆alkylOR⁶, fluoromethyl, difluoromethyl, trifluoromethyl, fluoromethoxy, difluoromethoxy, trifluoromethoxy, C₀₋₆alkylNR⁶R⁷, OC₁₋₆alkylNR¹⁶R⁷, OC₁₋₆alkylOC₁₋₆alkylNR⁶R⁷, NR⁶OR⁷, C₀₋₆alkylCO₂R⁶, OC₁₋₆alkylCO₂R⁶, C₀₋₆alkylCONR⁶R⁷, OC₁₋₆alkylCONR⁶R⁷, OC₁₋₆alkylNR⁶ (CO)R⁷, C₀₋₆alkylNR⁶ (CO)R⁷, O(CO)NR⁶R⁷, NR⁶ (CO)OR⁷, NR⁶(CO)NR⁶R⁷, O(CO)OR⁶, O(CO)R⁶, C₀₋₆alkylCOR⁶, OC₁₋₆alkylCOR⁶, NR⁶ (CO) (CO)R⁶, NR⁶(CO) (CO)NR⁶R⁷, SR⁶, C₀₋₆alkyl(SO₂)NR⁶R⁷, OC₁₋₆alkylNR⁶(SO₂)R⁷, OC₀₋₆alkyl (SO₂)NR⁶R⁷, C₀₋₆alkyl (SO)NR⁶R⁷, OC₁₋₆alkyl (SO)NR⁶R⁷, SO₃R⁶, C₀₋₆alkylNR⁶ (SO₂)NR⁶R⁷, C₀₋₆alkylNR⁶ (SO)R⁷, OC₁l₆alkylNR⁶ (SO)R⁷ OC₀₋₆alkylSO₂R⁶, C₀₋₆alkylSO₂R⁶, C₀₋₆alkylSOR⁶, C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl may be is optionally substituted by one or more A;
m is 0, 1, 2, 3 or 4;
n is 0, 1, 2, 3 or 4;
R⁵, is hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₁₋₆alkylNR⁶R⁷ or C₁₋₆alkylCONR⁶R⁷;
R⁶ and R⁷ are independently selected from hydrogen, C₁₋₆alkyl, (CO)OR¹, C₂₋₆alkenyl, C₂₋₆alkynyl, C₀₋₆alkylC₃₋₆cycloalkyl, C₀₋₆alkylaryl, C₀₋₆alkylheteroaryl and C₁₋₆alkylNR⁸R⁹;
R⁶ and R⁷ may together form a substituted 5- or 6-membered heterocyclic ring containing one or more heteroatoms selected from N, O and S, which heterocyclic ring is optionally substituted by A;
R⁸ and R⁹ are independently selected from hydrogen, C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl;
R⁸ and R⁹ may together form a 5- or 6-membered heterocyclic ring containing one or more heteroatoms selected from N, O and S, which heterocyclic ring is optionally substituted by A;
R¹⁰ is hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl or C₁₋₆alkylNR⁸R^{9;}
R¹¹ is C₁₋₆alkylNR⁸R⁹ or C₀₋₆alkylheterocycloalkyl;
R¹⁰ and R¹¹ may together form a 5-, 6- or 7-membered heterocyclic ring containing one or more heteroatoms selected from N, O or and S, which heterocyclic ring may be optionally substituted by A;
R¹² is a 5-, 6- or 7-membered heterocyclic ring containing one or more heteroatoms selected from N, O and S, which heterocyclic ring is optionally substituted by A;
wherein any C₁₋₆alkyl, C₂₋₆alkenyl, C₂₋₆alkynyl, C₀₋₆alkylC₃₋₆cycloalkyl, C₀₋₆alkylheterocycloalkyl, C₀₋₆alkylaryl, and C₀₋₆alkylheteroaryl defined under R⁵ to R¹² may be substituted by one or more A;
A is halo, nitro, CHO, CN, OR⁶, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR⁶R⁷, OC₆alkylNR⁶R⁷, CO₂R⁸, CONR⁶R⁷, NR⁶ (CO)R¹, O(CO)R⁶, COR⁶, SR⁶, (SO₂)NR⁶R⁷, (SO)NR⁶R⁷, SO₃R⁶, SO₂R⁶or SOR⁶, with the proviso that the compound is not 4-[4-[5-amino-6-(phenylmethyl)pyrazinyl]phenoxy]-ethyl ester butanoic acid.

2. A compound having the formula I 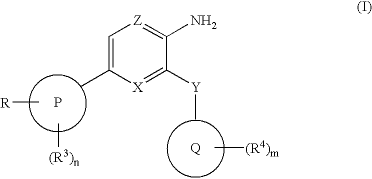
as a free base or a pharmaceutically acceptable salt thereof,
wherein:
Z is N;
Y is CONR⁵, NR⁵CO, SO₂NR⁵, NR⁵SO₂, CH₂NR⁵, NR⁵CH₂, NR⁵CONR⁵, CH₂CO, COCH2, CH═CH, OCH₂ or CH₂O;
X is CH or N;
P is phenyl or a 5- or 6-membered heteroaromatic ring containing one or more heteroatoms selected from N, O and S and said phenyl ring or 5- or 6-membered heteroaromatic ring is optionally be fused with a 5- or 6-membered saturated, partially saturated or unsaturated ring containing one or more atoms selected from C, N, O and S;
Q is phenyl or a 5- or 6-membered heteroaromatic ring containing one or more heteroatoms selected from N, O or and S of which at least one atom is selected from nitrogen;
R is CHO, fluoromethoxy, difluoromethoxy, trifluoromethoxy, C₀₋₆alkyl(SO₂)NR¹R¹², OC₀₋₆alkyl(SO₂)NR¹R², OC₁₋₆alkyl(SO)NR¹R¹² C₁₋₆alkyl(SO)NR¹R¹², C₀₋₆alkylNR²¹(SO)R¹, OC₁₋₆alkylNR² (SO)RC₀₋₆alkylNR¹(SO₂) NR¹R¹, OC₁₋₆alkylNR¹(SO₂) RC₀₋₆alkyl (SO₂) C₁₋₆alkylNR¹R², OC₀₋₆alkyl (SO₂) C₁₋₆alkylNR¹R¹² C₀₋₆alkyl (SO) C₁₋₆alkylNRPR², OC₁₋₆alkyl (SO) C₁₋₆alkylNR¹R^{2, C}₀₋₆alkylSC₁₋₆alkylNRPR¹², OC₁₋₆alkylSC₁₋₆alkylNR¹R¹² OC₁₋₆alkylOC₁₋₆alkyl, C₁₋₆alkylOC₁₋₆alkylNR¹R², OC₁₋₆alkylOC₁₋₆alkylNR¹R², C₀₋₆alkylCONR¹OR¹¹, OC₀₋₆alkylCONR¹R², OC₁₋₆alkylNR¹²R¹⁰, C₀₋₆alkylNR¹(CO)R”, OC₁₋₆alkylNR¹(CO)RC₀₋₆alkylNR¹¹(CO)R¹⁰, C₀₋₆alkylCOR¹¹, OC₁₋₆alkylCOR¹, C₀₋₆alkylNR¹OR¹¹, C₀₋₆alkylO(CO)R¹¹, OC₁₋₆alkylO(CO)R¹, C₀₋₆alkylC(NR¹⁰)NR¹⁰R¹¹, C₀₋₆alkylC(NR”)N(R¹⁰)₂, OC₀₋₆alkylC(NR¹)NR¹R¹, C₀₋₆alkylNR¹(CO)OR¹¹, OC₁₋₆alkylNR¹(CO)OR¹, C₀₋₆alkylNR¹¹(CO)OR¹⁰, OC₁₋₆alkylCN, NR⁶OR⁷C₀₋₆alkyl(CO)OR¹, OC₁₋₆alkyl(CO)OR¹, NR¹(CO)NR¹R², NR¹(CO)(CO)RNR¹(CO) (CO)NR¹R² or SO₃RL;
R¹ and R² are independently selected from hydrogen, C₁₋₆alkyl, C₂₋₆alkenyl, C₂₋₆alkynyl, C₀₋₆alkylC₃₋₆cycloalkyl, C₁₋₆alkylNR⁶R⁷, C₀₋₆alkylaryl and C₀₋₆alkylheteroaryl, wherein any C₁₋₆alkyl, C₂₋₆-alkenyl, C₂₋₆alkynyl, C₀₋₆alkylC₃₋₆cycloalkyl, C₀₋₆alkylaryl, and C₀₋₆alkylheteroaryl may be substituted by one or more A;
R¹ and R² may together form a substituted 5- or 6-membered heterocyclic ring containing one or more heteroatoms selected from N, O and S, and if said heterocyclic ring contains an —NH— moiety that ring nitrogen is optionally substituted by A;
R³ and R⁴ are independently selected from halo, nitro, CHO, C₀₋₆alkylCN, OC₁₋₆alkylCN, C₀₋₆alkylOR⁶, OC₁₋₆alkylOR⁶, fluoromethyl, difluoromethyl, trifluoromethyl, fluoromethoxy, difluoromethoxy, trifluoromethoxy, C₀₋₆alkylNR⁶R⁷, OC₁₋₆alkylNR⁶R⁷, OC₁₋₆alkylOC₁₋₆alkylNR¹⁶R⁷, NR⁶OR⁷ C₀₋₆alkylCO₂R⁶, OC₁₋₆alkylCO₂R⁶, C₀₋₆alkylCONR⁶R⁷, OC₁₋₆alkylCONR⁶R⁷, OC₁₋₆alkylNR⁶ (CO)R⁷, C₀₋₆alkylNR⁶ (CO)R⁷, O(CO)NR⁶R⁷, NR⁶(CO)OR⁷, NR⁶ (CO)NR⁶R⁷, O(CO)OR⁶, O(CO)R⁶, C₀₋₆alkylCOR⁶, OC₁₋₆alkylCOR⁶, NR⁶ (CO) (CO)R⁶, NR⁶ (CO) (CO)NR⁶R⁷, SR⁶, C₀₋₆alkyl(SO₂)NR⁶R⁷, OC₁₋₆alkylNR⁶(SO₂)R⁷, OC₀₋₆alkyl(SO₂)NR⁶R⁷, C₀₋₆alkyl(SO)NR¹⁶R⁷, OC₁₋₆alkyl (SO)NR⁶R⁷, SO₃R⁶, C₀₋₆alkylNR⁶(SO₂)NR⁶R⁷, C₀₋₆alkylNR⁶ (SO)R⁷, OC₁₋₆alkylNR⁶(SO)R⁷, OC₀₋₆alkylSO₂R⁶, C₀₋₆alkylSO₂R⁶, C₀₋₆alkylSOR⁶, C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl is optionally substituted on any carbon atom by one or more A and if said heteroaryl contains an —NH— moiety that nitrogen is optionally substituted by A;
m is 0, 1, 2, 3 or 4;
n is 0, 1, 2, 3 or 4;
R⁵ is hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₁₋₆alkylNR⁶R⁷ or C₁₋₆alkylCONR⁶R⁷;
R⁶ and R⁷ are independently selected from hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl and C₁₋₆alkylNR⁸R⁹;
R⁶ and R⁷ may together form a substituted 5- or 6-membered heterocyclic ring containing one or more heteroatoms selected from N, O er and S, and if said heterocyclic ring contains an —NH— moiety that ring nitrogen may be is optionally substituted by A;
R⁸ and R⁹ are independently selected from hydrogen, C₁₋₆alkyl, C₂₋₆alkenyl, C₂₋₆alkynyl, C₀₋₆alkylC₃₋₅₆cycloalkyl, C₀₋₆alkylaryl and C₀₋₆alkylheteroaryl;
R⁸ and R⁹ may together form a 5- or 6-membered heterocyclic ring containing one or more heteroatoms selected from N, O and S, and if said heterocyclic ring contains an —NH— moiety that ring nitrogen is optionally substituted by A;
R¹⁰ is hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl or C₁₋₆alkylNRBR^{9;}
R¹¹ is C₁₋₆alkylNR⁸R^{9;}
R¹⁰ and R¹¹ may together form a 5- or 6-membered heterocyclic ring containing one or more heteroatoms selected from N, O or and S, and if said heterocyclic ring contains an —NH— moiety that ring nitrogen may be is optionally substituted by A; wherein any C₁₋₆alkyl, C₂₋₆alkenyl, C₂₋₆alkynyl, C₀₋₆alkylC₃₋₆cycloalkyl, C₀₋₆alkylaryl, and C₀₋₆alkylheteroaryl defined under R⁵ to R¹¹ may be substituted by one or more A;
A is halo, nitro, CHO, CN, OR⁶, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR⁶R⁷, OC₁₋₆alkylNR¹⁶R⁷, CO₂R⁶, CONR⁶R⁷, NR¹(CO)R¹, O(CO)R¹, COR, SR¹, (SO₂)NR¹R¹, (SO)NR¹R¹, SO₃R⁶, SO₂R⁶ or SOR⁶.

3. A compound according to claim 1, wherein:
Z is CH or N;
Y is CONR⁵;
X is CH or N;
P is phenyl or a 5-membered heteroaromatic ring containing one heteroatom selected from O or S;
Q is a 6-membered heteroaromatic ring containing one heteroatom selected from N;
112 R¹² is C₀₋₆alkyl (SO₂) NR¹OR¹, C₀₋₆alkylCONR¹R¹, OC₁₋₆alkylNR¹R², C₀₋₆alkyl(CO)OR⁸ or OR¹²;
R¹ and R² are independently selected from hydrogen, C₁₋₆alkyl, (CO)OR⁸, C₀₋₆alkylheterocycloalkyl, C₁₋₆alkylNR⁶R⁷ and C₀₋₆alkylheteroaryl, wherein any C₁₋₆alkyl or C₀₋₆alkylheterocycloalkyl may be substituted by one or more A;
R¹ and R² may together form a substituted 5-, 6- or 7-membered heterocyclic ring containing one or more heteroatoms selected from N and O, which heterocyclic ring is optionally substituted by A;
R³ and R⁴ are independently selected from halo, trifluoromethyl, trifluoromethoxy, C₀₋₆alkylNR⁶R⁷ and C₁₋₆alkyl;
m is 0 or 1;
n is 0, 1 or 2;
R⁵ is hydrogen;
R⁶ and R⁷ are independently selected from hydrogen, C₁₋₆alkyl and (CO)OR⁸;
R⁶ and R⁷ may together form a substituted 5- or 6-membered heterocyclic ring containing one or more heteroatoms selected from N, which heterocyclic ring is optionally substituted by A;
R⁸ and R⁹ are independently selected from hydrogen and C₁₋₆alkyl;
R⁸ and R⁹ may together form a 5- or 6-membered heterocyclic ring containing one or more heteroatoms selected from N or O, which heterocyclic ring may be is optionally substituted by A;
R¹⁰ is hydrogen or C₁₋₆alkyl;
R¹¹ is C₁₋₆alkylNR⁸R⁹ or C₀₋₆alkylheterocycloalkyl;
R¹⁰ and R¹¹ may together form a 5-, 6- or 7-membered heterocyclic ring containing one or more heteroatoms selected from N, which heterocyclic ring is optionally substituted by A;
R¹² is a 5-, 6- or 7-membered heterocyclic ring containing one or more heteroatoms selected from N, O es and S, which heterocyclic ring may be is optionally substituted by A;
wherein C₀₋₆alkylheterocycloalkyl defined under R⁵ to R¹² may be substituted by one or more A;
A is OR⁶, C₁₋₆alkyl, C₀₋₆alkylNR⁶R⁷, COR⁶ or CO₂R⁸.

4. A compound according to claim 1, wherein Y is CONR⁵.

5. A compound according to claim 1, wherein P is phenyl.

6. A compound according to claim 1, wherein P is a 5- or 6-membered heteroaromatic ring containing one or more heteroatoms selected from N, O and S.

7. A compound according to claim 6, wherein P is furan or thiophene.

8. A compound according to claim 1, wherein Q is pyridine.

9. A compound according to claim 1, wherein R is C₀₋₆alkyl(SO₂)NR¹R².

10. A compound according to claim 9, wherein R is (SO₂) NR¹R¹².

11. A compound according to claim 1, wherein R is OC₁₋₆alkylNR¹R².

12. A compound according to claim 1, wherein R is in the 4 position.

13. A compound which is
3-Amino-6-{4-[(dimethylamino)sulfonyl]phenyl}-N-pyridin-3-ylpyrazine-2-carboxamide,
3-Amino-6-{3-[(dimethylamino)sulfonyl]phenyl}-N-pyridin-3-ylpyrazine-2-carboxamide,
3-Amino-6-{2-[(dimethylamino)sulfonyl]phenyl}-N-pyridin-3-ylpyrazine-2-carboxamide,
3-Amino-6-[4-(aminosulfonyl)phenyl]-N-pyridin-3-ylpyrazine-2-carboxamide,
2-Amino-5-{4-[(dimethylamino)sulfonyl]phenyl}-N-pyridin-3-ylnicotinamide, or
3-Amino-6-(4-{[(3-morpholin-4-ylpropyl)amino]sulfonyl}phenyl)-N- pyridin-3-ylpyrazine-2-carboxamide as a free base or a pharmaceutically acceptable salt thereof.

14. A compound which is
3-Amino-6-[4-[2-(4-methyl-1-piperazinyl)ethoxy]phenyl]-N—.(3-pyridinyl)-2-pyrazinecarboxamide as a free base or a pharmaceutically acceptable salt thereof.

15. A compound which is
3-Amino-N-pyridin-3-yl-6-[4-(pyrrolidin-1-ylsulfonyl)phenyl]pyrazine-2-carboxamide,
3-Amino-6-[4-(piperidin-1-ylsulfonyl)phenyl]-N-pyridin-3-ylpyrazine-2-carboxamide,
3-Amino-6-{3-ethyl-4-[(4-methylpiperazin-1-yl)sulfonyl]phenyl}-N-pyridin-3-ylpyrazine-2-carboxamide,
3-Amino-N-{5-[3-(dimethylamino)propyl]pyridin-3-yl}-6-[4-(piperidin-1-ylsulfonyl)phenyl]pyrazine-2-carboxamide,
3-Amino-N-{5-[3-(dimethylamino)propyl]pyridin-3-yl}-6-[4-(pyrrolidin-1-ylsulfonyl)phenyl]pyrazine-2-carboxamide,
3-Amino-N-{4-[(dimethylamino)methyl]pyridin-3-yl}-6-{4-[(dimethylamino)sulfonyl]phenyl}pyrazine-2-carboxamide,
3-Amino-N-{4-[3-(dimethylamino)propyl]pyridin-3-yl}-6-{4-[(dimethylamino)sulfonyl]phenyl}pyrazine-2-carboxamide,
3-Amino-6-[4-(morpholin-4-ylsulfonyl)phenyl]-N-pyridin-3-ylpyrazine-2-carboxamide,
3-Amino-6-{4-[(4-ethylpiperazin-1-yl)sulfonyl]phenyl}-N-pyridin-3-ylpyrazine-2-carboxamide,
3-Amino-N-pyridin-3-yl-6-(4-{[(2-pyridin-2-yltethyl)amino]sulfonyl}phenyl)pyrazine-2-carboxamide,
3-Amino-6-[4-({[2-(dimethylamino)-1-methylethyl]amino}sulfonyl)phenyl]-N-pyridin-3-ylpyrazine-2-carboxamide,
3-Amino-N-pyridin-3-yl-6-(4-{[(3-pyrrolidin-1-ylpropyl)amino]sulfonyl}phenyl)pyrazine-2-carboxamide,
6-{4-[(4-Acetylpiperazin-1-yl)sulfonyl]phenyl}-3-amino-N- pyridin-3-ylpyrazine-2-carboxamide,
2-Amino-5-{4-[(4-methylpiperazin-1-yl)sulfonyl]phenyl}-N-[4-(pyrrolidin-1-ylmethyl)pyridin-3-yl]nicotinamide,
3-Amino-6-(4-{[[2-(dimethylamino)ethyl](ethyl)amino]carbonyl}phenyl)-N-pyridin-3-ylpyrazine-2-carboxamide,
3-Amino-6-(4-{[[3-(dimethylamino)propyl](methyl)amino]carbonyl}phenyl)-N-pyridin-3-ylpyrazine-2-carboxamide,
3-Amino-6-[4-({[3-(dimethylamino)propyl]amino}carbonyl)phenyl]-N-pyridin-3-ylpyrazine-2-carboxamide,
-3-Amino-N-pyridin-3-yl-6-(4-{[(2-pyrrolidin-1-ylethyl)amino]carbonyl}phenyl)pyrazine-2-carboxamide,
3-Amino-N-pyridin-3-yl-6-(4-{[(3-pyrrolidin-1-ylpropyl)amino]carbonyl}phenyl)pyrazine-2-carboxamide,
3-Amino-6-[4-({[2-(dimethylamino)ethyl]amino}carbonyl)phenyl]-N- pyridin-3-ylpyrazine-2-carboxamide,
3-Amino-6-[4-({[2-(dimethylamino)-1-methylethyl]amino}carbonyl)phenyl]-N-pyridin-3-ylpyrazine-2-carboxamide,
3-Amino-6-[4-({[3-(4-methylpiperazin-1-yl)propyl]amino}carbonyl)phenyl]-N-pyridin-3-ylpyrazine-2-carboxamide,
3-Amino-6-(4-{[(2-piperidin-1-ylethyl)amino]carbonyl}phenyl)-N- pyridin-3-ylpyrazine-2-carboxamide,
3-Amino-N-pyridin-3-yl-6-{4-[(4-pyrrolidin-1-ylpiperidin-1-yl)carbonyl]phenyl}pyrazine-2-carboxamide,
4-Amino-4′-[(4-methylpiperazin-1-yl)sulfonyl]-N-pyridin-3-yl-1,1′-biphenyl-3-carboxamide,
3-Amino-6-[4-[[[2-(4-morpholinyl)ethyl]amino]carbonyl]phenyl}-N-(3-pyridinyl)-2-pyrazinecarboxamide,
tert-Butyl 4-[2-(4-{5-amino-6-[(pyridin-3-ylamino)carbonyl]pyrazin-2-yl}phenoxy)ethyl]piperazine-1-carboxylate,
tert-Butyl 4-[2-(4-{5-amino-6-[(pyridin-3-ylamino)carbonyl]pyrazin-2-yl}-2,5-difluorophenoxy)ethyl]piperazine-1-carboxylate,
3-Amino-6-{5-[(dimethylamino)sulfonyl]thien-2-yl}-N-pyridin-3-ylpyrazine-2-carboxamide,
tert-Butyl 4-(5-{5-amino-6-[(pyridin-3-ylamino)carbonyl]pyrazin-2-yl}-2-furoyl)piperazine-1-carboxylate,
3-Amino-6-[4-{[(2-aminoethyl)amino]sulfonyl}-3-(trifluoromethoxy)phenyl]-N-pyridin-3-ylpyrazine-2-carboxamide,
-3-Amino-6-[4-({[2-(dimethylamino)ethyl]amino}sulfonyl)phenyl]-N- pyridin-3-ylpyrazine-2-carboxamide or 4-{5-Amino-6-[(pyridin-3-ylamino)carbonyl]pyrazin-2-yl}benzoic acid,
as a free base or a pharmaceutically acceptable salt thereof, or
3-Amino-6-(4-{[[3-(dimethylamino)propyl](methyl)amino]sulfonyl}phenyl)-N-pyridin-3-ylpyrazine-2-carboxamide hydrochloride,
3-Amino-6-[4-({[3-(4-methylpiperazin-1-yl)propyl]amino}sulfonyl)phenyl]-N-pyridin-3-ylpyrazine-2-carboxamide hydrochloride,
3-Amino-N-pyridin-3-yl-6-(4-{[(2-pyrrolidin-1-ylethyl)amino]sulfonyl}phenyl)pyrazine-2-carboxamide-hydrochloride,
3-Amino-6-[4-({[2-(dimethylamino)propyl]amino}sulfonyl)phenyl]-N-pyridin-3-ylpyrazine-2-carboxamide hydrochloride,
3-Amino-6-(4-{[isopropyl(2-methoxyethyl)amino]sulfonyl}phenyl)-N-pyridin-3-ylpyrazine-2-carboxamide hydrochloride,
3-Amino-6-[4-({[2-(diethylamino)ethyl]amino}sulfonyl)phenyl]-N-pyridin-3-ylpyrazine-2-carboxamide hydrochloride,
3-Amino-6-(4-{[[2-(dimethylamino)ethyl](ethyl)amino]sulfonyl}phenyl)-N-pyridin-3-ylpyrazine-2-carboxamide hydrochloride,
3-Amino-6-[4-({[3-(dimethylamino)propyl]amino}sulfonyl)phenyl]-N-pyridin-3-ylpyrazine-2-carboxamide hydrochloride,
3-Amino-6-{3-methyl-4-[(4-methylpiperazin-1-yl)sulfonyl]phenyl}-N-pyridin-3-ylpyrazine-2-carboxamide hydrochloride,
3-Amino-6-{2-[(4-methylpiperazin-1-yl)sulfonyl]phenyl}-N-pyridin-3-ylpyrazine-2-carboxamide hydrochloride,
3-Amino-6-{3-[(4-methylpiperazin-1-yl)sulfonyl]phenyl}-N-pyridin-3-ylpyrazine-2-carboxamide hydrochloride,
3-Amino-6-{2-methyl-4-[(4-methylpiperazin-1-yl)sulfonyl]phenyl}-N-pyridin-3-ylpyrazine-2-carboxamide hydrochloride,
3-Amino-6-[4-({[2-(dimethylamino)ethyl]amino}sulfonyl)-3-(trifluoromethoxy)phenyl]-N-pyridin-3-ylpyrazine-2-carboxamide hydrochloride,
3-Amino-6-[4-{[[2-(dimethylamino)ethyl](ethyl)amino]sulfonyl}-3-(trifluoromethoxy)phenyl]-N-pyridin-3-ylpyrazine-2-carboxamide hydrochloride,
3-Amino-6-[4-[(4-methylpiperazin-1-yl)sulfonyl]-2-(trifluoromethyl)phenyl]-N-pyridin-3-ylpyrazine-2-carboxamide hydrochloride,
3-Amino-6-[4-[2-(dimethylamino)ethoxy]phenyl]-N-(3-pyridinyl)-2-pyrazine-carboxamide hydrochloride,
3-Amino-6-[4-[2-(4-morpholinyl) ethoxy]phenyl]-N-(3-pyridinyl)-2-pyrazinecarboxamide hydrochloride,
3-Amino-6-[4-[[[2-(dimethylamino)ethyl]methylamino]carbonyl]phenyl}-N-(3-pyridinyl)-2-pyrazinecarboxamide hydrochloride,
3-Amino-6-{2-fluoro-4-[(4-methylpiperazin-1-yl)sulfonyl]phenyl}-N-pyridin-3-ylpyrazine-2-carboxamide hydrochloride,
3-Amino-6-{5-fluoro-2-methyl-4-[(4-methylpiperazin-1-yl)sulfonyl]phenyl}-N-pyridin-3-ylpyrazine-2-carboxamide hydrochloride,
3-Amino-6-{2,5-dimethyl-4-[(4-methylpiperazin-1-yl)sulfonyl]phenyl}-N-pyridin-3-ylpyrazine-2-carboxamide hydrochloride,
3-Amino-6-[4-(2-piperidin-1-ylethoxy)phenyl]-N-pyridin-3-ylpyrazine-2-carboxamide hydrochloride,
3-Amino-6-[4-(2-pyrrolidin-1-ylethoxy)phenyl]-N-pyridin-3-yl-pyrazine-2-carboxamide hydrochloride,
-3-Amino-6-[2,5-difluoro-4-(2-morpholin-4-ylethoxy)phenyl]-N-pyridin-3-ylpyrazine-2-carboxamide hydrochloride,
3-Amino-6-{4-[(4-methylpiperazin-1-yl)carbonyl]phenyl}-N-pyridin-3-ylpyrazine-2-carboxamide hydrochloride,
3-Amino-6-[2,5-difluoro-4-(2-pyrrolidin-1-ylethoxy)phenyl]-N-pyridin-3-ylpyrazine-2-carboxamide hydrochloride,
3-Amino-6-{2,6-dimethyl-4-[2-(4-methylpiperazin-1-yl)ethoxy]phenyl}-N-pyridin-3-ylpyrazine-2-carboxamide hydrochloride,
3-Amino-6-{2-methyl-4-[2-(4-methylpiperazin-1-yl)ethoxy]phenyl}-N-pyridin-3-ylpyrazine-2-carboxamide hydrochloride,
2-Amino-5-{4-[(4-methylpiperazin-1-yl)sulfonyl]phenyl}-N-pyridin-3-ylnicotinamide hydrochloride,
3-Amino-6-{4-[(4-methylpiperazin-1-yl)sulfonyl]phenyl}-N-[4-(pyrrolidin-1-ylmethyl)pyridin-3-yl]pyrazine-2-carboxamide hydrochloride,
3-Amino-6-[2,5-difluoro-4-(pyrrolidin-1-ylsulfonyl)phenyl]-N-[4-(2-pyrrolidin-1-ylethyl)pyridin-3-yl]pyrazine-2-carboxamide hydrochloride,
3-Amino-6-[2,5-difluoro-4-(pyrrolidin-1-ylsulfonyl)phenyl]-N-[5-(3-pyrrolidin-1-ylpropyl)pyridin-3-yl]pyrazine-2-carboxamide hydrochloride,
3-Amino-6-[2,5-difluoro-4-(piperidin-1-ylsulfonyl)phenyl]-N-[5-(3-pyrrolidin-1-ylpropyl)pyridin-3-yl]pyrazine-2-carboxamide hydrochloride,
3-Amino-6-[4-(piperidin-1-ylsulfonyl)phenyl]-N-[5-(3-pyrrolidin-1-ylpropyl)pyridin-3-yl]pyrazine-2-carboxamide hydrochloride,
3-Amino-N-[5-(3-pyrrolidin-1-ylpropyl)pyridin-3-yl]-6-[4-(pyrrolidin-1-ylsulfonyl)phenyl]pyrazine-2-carboxamide hydrochloride,
-3-Amino-N-[4-(2-pyrrolidin-1-ylethyl)pyridin-3-yl]-6-[4-(pyrrolidin-1-ylsulfonyl)phenyl]pyrazine-2-carboxamide hydrochloride,
3-Amino-N-[4-(3-pyrrolidin-1-ylpropyl)pyridin-3-yl]-6-[4-(pyrrolidin-1-ylsulfonyl)phenyl]pyrazine-2-carboxamide hydrochloride,
3-Amino-N-[4-(pyrrolidin-1-ylmethyl)pyridin-3-yl]-6-[4-(pyrrolidin-1-ylsulfonyl)phenyl]pyrazine-2-carboxamide hydrochloride,
3-Amino-N-{4-[(dimethylamino)methyl]pyridin-3-yl}-6-[4-(pyrrolidin-1-ylsulfonyl)phenyl]pyrazine-2-carboxamide hydrochloride,
3-Amino-N-{4-[(dimethylamino)methyl]pyridin-3-yl}-6-[4-(piperidin-1-ylsulfonyl)lphenyl]pyrazine-2-carboxamide hydrochloride,
3-Amino-6-{3-ethyl-4-[(4-methylpiperazin-1-yl)sulfonyl]phenyl}-N-pyridin-3-ylpyrazine-2-carboxamide hydrochloride,
3-Amino-6-[4-[(4-methylpiperazin-1-yl)sulfonyl]-3-(trifluoromethoxy)phenyl]-N-pyridin-3-ylpyrazine-2-carboxamide hydrochloride,
0.3-Amino-6-[4-{[(2-aminoethyl)amino]sulfonyl}-3-(trifluoromethoxy)phenyl]-N-pyridin-3-ylpyrazine-2-carboxamide hydrochloride,
4-Amino-4′-[(4-methylpiperazin-1-yl)sulfonyl)-N-pyridin-3-yl-1,1′-biphenyl-3-carboxamide hydrochloride,
2-Amino-5-{4-[(4-methylpiperazin-1-yl)sulfonyl]phenyl}-N-[4-(pyrrolidin-1-ylmethyl)pyridin-3-yl]nicotinamide hydrochloride,
3-Amino-N-pyridin-3-yl-6-[4-(pyrrolidin-1-ylsulfonyl)phenyl]pyrazine-2-carboxamide hydrochloride,
3-Amino-6-[4-(piperidin-1-ylsulfonyl)phenyl]-N-pyridin-3-ylpyrazine-2-carboxamide hydrochloride,
-3-Amino-6-[4-(piperazin-1-ylsulfonyl)phenyl]-N-pyridin-3-ylpyrazine-2-carboxamide hydrochloride,
3-Amino-6-[4-(2-piperazin-1-ylethoxy)phenyl]-N-pyridin-3-ylpyrazine-2-carboxamide hydrochloride,
3-Amino-6-[2,5-difluoro-4-(2-piperazin-1-ylethoxy)phenyl]-N-pyridin-3-ylpyrazine-2-carboxamide hydrochloride,
3-Amino-6-[5-(piperazin-1-ylcarbonyl)-2-furyl]-N-pyridin-3-ylpyrazine-2-carboxamide hydrochloride or 3-Amino-N-{5-[3-(dimethylamino)propyl]pyridin-3-yl}-6-[4-(piperidin-1-ylsulfonyl)phenyl]pyrazine-2-carboxamide hydrochloride.

16. A compound which is
tert-Butyl 4-[(4-{5-amino-6-[(pyridin-3-ylamino)carbonyl]pyrazin-2-yl}phenyl)sulfonyl]piperazine-1-carboxylate,
3-Amino-6-(4-{[methyl(1-methylpyrrolidin-3-yl)amino]sulfonyl}phenyl)-N-pyridin-3-ylpyrazine-2-carboxamide,
3-Amino-6-(4-{[methyl(1-methylpiperidin-4-yl)amino]sulfonyl}phenyl)-N-pyridin-3-ylpyrazine-2-carboxamide,
3-Amino-6-(4-{[3-(dimethylamino)pyrrolidin-1-yl]sulfonyl}phenyl)-N-pyridin-3-ylpyrazine-2-carboxamide,
3-Amino-6-{4-[(4-methyl-1,4-diazepan-1-yl)carbonyl]phenyl}-N-pyridin-3-ylpyrazine-2-carboxamide,
3-Amino-6-(4-{[methyl(1-methylpyrrolidin-3-yl)amino]carbonyl}phenyl)-N-pyridin-3-ylpyrazine-2-carboxamide,
3-Amino-6-(4-{[3-(dimethylamino)pyrrolidin-1-yl]carbonyl}phenyl)-N-pyridin-3-ylpyrazine-2-carboxamide,
3-Amino-6-[4-({[(1-ethylpyrrolidin-2-yl)methyl]amino}carbonyl)phenyl]-N-pyridin-3-ylpyrazine-2-carboxamide,
-3-Amino-6-(4-{[methyl(1-methylpiperidin-4-yl)amino]carbonyl}phenyl)-N-pyridin-3-ylpyrazine-2-carboxamide,
3-Amino-6-(4-{[(1-ethylpiperidin-3-yl)amino]carbonyl}phenyl)-N-pyridin-3-ylpyrazine-2-carboxamide,
3-Amino-6-[4-({(2-(1-methylpyrrolidin-2-yl)ethyl]amino}carbonyl)phenyl]-N-pyridin-3-ylpyrazine-2-carboxamide,
tert-Butyl 2-{[(4-{5-amino-6-[(pyridin-3-ylamino)carbonyl]pyrazin-2-yl}phenyl)sulfonyl]-(tert-butoxycarbonyl)amino}ethylcarbamate or
3-Amino-6-[4-[(1-methyl-3-pyrrolidinyl)oxy]phenyl]-N-(3—pyridinyl)-2-pyrazinecarboxamide,
as a free base or a pharmaceutically acceptable salt thereof, or
3-Amino-6-{4-[(4-methyl-1,4-diazepan-1-yl)sulfonyl]phenyl}-N-pyridin-3-ylpyrazine-2-carboxamide hydrochloride or
3-Amino-6-[4-({[(1-ethylpyrrolidin-2-yl)methyl]amino}sulfonyl)phenyl]-N-pyridin-3-ylpyrazine-2-carboxamide hydrochloride.

17. A pharmaceutical formulation for use in the prevention and/or treatment of conditions associated with glycogen synthase kinase-3 comprising as active ingredient a therapeutically effective amount of the compound of any one of claims 1 to 16 and 43 to 51 in association with one or more pharmaceutically acceptable diluents, excipients and/or inert carriers.

18-30. (canceled)

31. A method of prevention and/or treatment of conditions associated with glycogen synthase kinase-3, comprising administering to a mammal in need of such prevention and/or, treatment a therapeutically effective amount of a compound of formula I as defined in any one of claims 1 to 16 and 43 to 51.

32. A method of prevention and/or treatment of Parkinson's Disease, Frontotemporal dementia Parkinson-s Type, Parkinson dementia complex of Gaum, HIV dementia, diseases with associated neurofibrillar tangle pathologies, amyotrophic lateral sclerosis, corticobasal degeneration, dementia pugilistica, Down syndrome, Huntington's Disease, postencephalitic parkinsonism, progressive supranuclear palsy, Niemann-Pick's Disease, Pick's Disease, stroke, head trauma and other chronic neurodegenative diseases, Bipolar Disorders, affective disorders, depression, schizophrenia, cognitive disorders, Type I and Type II diabetes, diabetic neuropathy, and hair loss or a method of contraceptive medication comprising administering to a mammal, in need of such prevention and/or treatment, or such medication, a therapeutically effective amount of a compound of formula I as defined in any one of claims 1 to 16 and 43 to 51.

33. A method of prevention and/or treatment of dementia or Alzheimer's Disease comprising administering to a mammal, in need of such prevention and/or, treatment a therapeutically effective amount of a compound of formula I as defined in any one of claims 1 to 16 and 43 to 51.

34. A method of prevention and/or treatment of diabetes comprising administering to a mammal, in need of such prevention and/or treatment a therapeutically effective amount of a compound of formula I as defined in any one of claims 1 to 16 and 43 to 51.

35. A process for the preparation of a compound of formula I, wherein Z, Y, X, P, Q, R, R¹, R², R³, R⁴, R⁵, R⁶, R⁷, R⁸, R⁹, R¹⁰, R¹¹, R¹², A, m and n are defined as in formula I according to any one of claims 1 to 3, comprising;
A) de-halogen coupling of a compound of formula XI with an aryl species to give a compound of formula I: 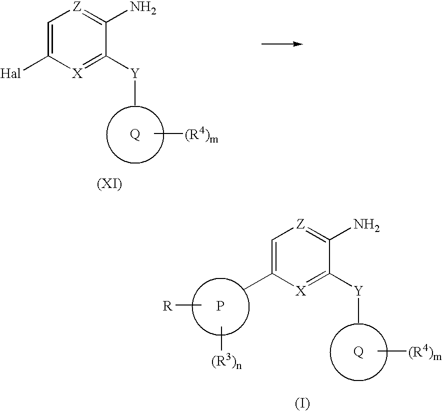
B) amidation of a compound of formula XIII with an appropriate amine: 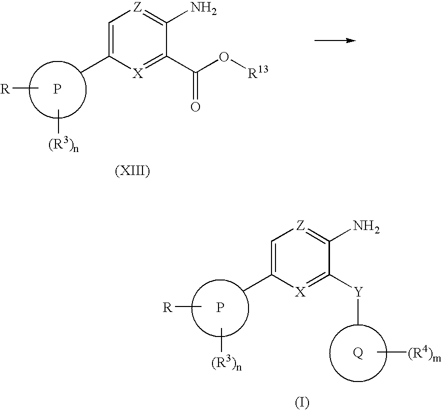
C) de-halogen coupling of a compound of formula XV with an aryl species to give a compound of formula I: 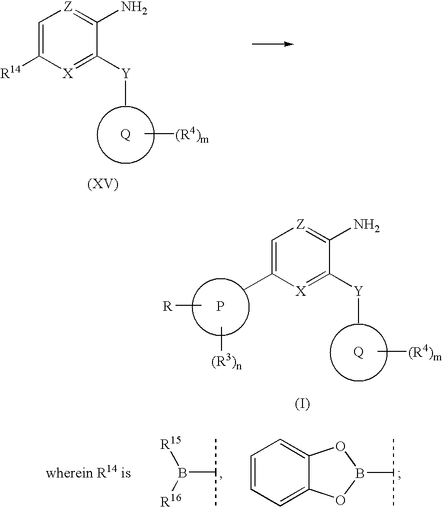
and
R¹⁵ and R^{1 6} are C₁₋₆alkyl or C₁₋₃alkyl fused together to form a 5- or 6-membered boron-oxygen-C₂-C₃cycloalkyl and the alkyl, cycloalkyl and the aryl moieties are optionally substituted;
D) reacting a compound of formula XVI, wherein L is a leaving group with an appropriate amine, to give a compound of formula Ia: 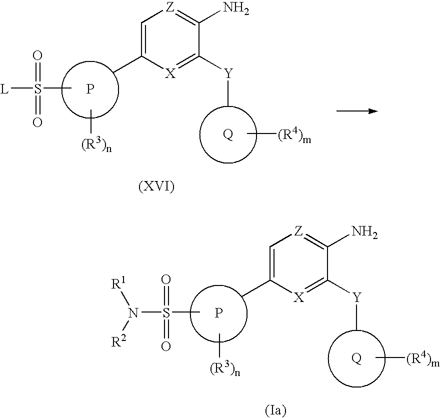
E) amidation of a compound of formula Ib, with the appropriate amine to give a compound of formula Ic: 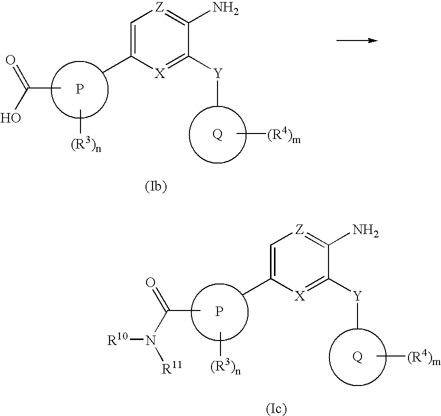
wherein the aryl species in routes A and C is selected from the group consisting of aryl halogen, aryl boronic acid and aryl stannane,
and the appropriate amine in routes B, D and E is selected from the group consisting of a compound of formula X 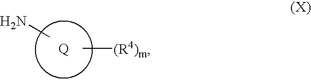
wherein Q is as defined above, R⁴ is C₁₋₆alkylNR⁶R⁷ and m is 1, HNR¹R², HNR¹⁰R¹¹ and 3-aminopyridine.

36. A compound of formula XI 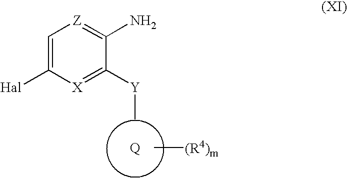
wherein Q is a 5- or 6-membered heteroaromatic ring containing one or more heteroatoms selected from N, O and S, of which at least one atom is selected from nitrogen and wherein Y, X, Z, Q R⁴, R⁵, R⁶, R⁷, R⁸, R⁹, A and m are defined as in formula I according to any one of claims 1 to 3.

37. A compound of formula XIII 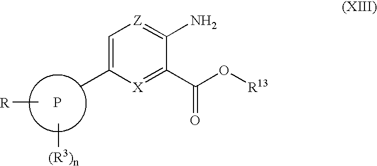
wherein X, Z, P, R¹, R², R³, R⁶, R⁷, R⁸, R⁹, R¹⁰, R¹¹, R¹², R¹, Aand n are defined as in formula I according to any one of claims 1 to 3 and R¹³ is hydrogen or C₁₋₆alkyl.

38. A compound of formula XV 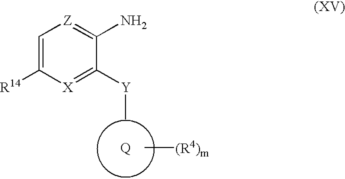
wherein Y, Z, X, Q, R⁴, R⁵, R⁶, R⁷, R⁸, R⁹, A and m are defined as in formula I according to any one of claims 1 to 3 and R¹⁴ is diethylboronate, 1,3,2-dioxaborolane, 1,3,2-dioxaborinane or 1,3,2-benzodioxaborole.

39. A compound of formula XVI 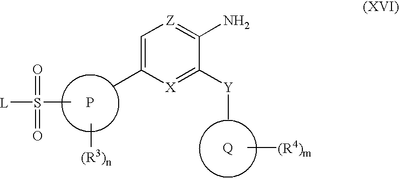
wherein Y, Z, X, P, Q, R³, R⁴, R⁵, R⁶, R⁷, R⁸, R⁹, A, m and n are defined as in formula I according to any one of claims 1 to 3 and L is a leaving group.

40. A compound which is:
N,N-Dimethyl-4-(4,4,5,5-tetramethyl-[1,3,2]-dioxaborolan-2-yl)benzenesulfonamide,
N,N-Dimethyl-3-(4,4,5,5-tetramethyl-1,3,2-dioxaborolan-2-yl)benzenesulfonamide,
N,N-Dimethyl-2-(4,4,5,5-tetramethyl-1,3,2-dioxaborolan-2-yl)benzenesulfonamide,
4-(4,4,5,5-Tetramethyl-1,3,2-dioxaborolan-2-yl)benzenesulfonamide,
4-{[(3-Morpholin-4-ylpropyl)amino]sulfonyl}phenylboronic acid,
4-((4-Methylpiperazin-1-yl)sulfonyl]phenylboronic acid,
4-Bromo-N-[2-(dimethylamino)ethyl]benzenesulfonamide or
4-Bromo-N-(3-morpholin-4-ylpropyl)benzenesulfonamide.

41. A compound which is:
1-[(4-Bromo-2,5-difluorophenyl)sulfonyl]-4-methylpiperazine,
1-[(4-Bromo-2-ethylphenyl)sulfonyl]-4-methylpiperazine,
1-{[4-Bromo-2-(trifluoromethoxy)phenyl]sulfonyl}-4-methylpiperazine,
1-[(4-Bromo-2-fluorophenyl)sulfonyl]-4-methylpiperazine,
1-[(4-Bromo-2-methylphenyl)sulfonyl]-4-methylpiperazine,
1-[(2-Bromophenyl)sulfonyl]-4-methylpiperazine,
1-[(3-Bromophenyl)sulfonyl]-4-methylpiperazine,
4-Bromo-N-[2-(dimethylamino)ethyl]-2-(trifluoromethoxy)benzenesulfonamide,
4-Bromo-N-[2-(dimethylamino)ethyl]-N-ethyl-2-(trifluoromethoxy)benzenesulfonamide,
N-(2-Aminoethyl)-4-bromo-2-(trifluoromethoxy)benzenesulfonamide,
tert-Butyl 2-({[4-bromo-2-(trifluoromethoxy)phenyl]sulfonyl}, (tert-butoxycarbonyl)amino)ethylcarbamate,
4-Bromo-N-methyl-N-(1-methylpyrrolidin-3-yl)benzenesulfonamide,
4-Bromo-N-[2-(dimethylamino)-1-methylethyl]benzenesulfonamide,
4-Bromo-N-(3-pyrrolidin-1-ylpropyl)benzenesulfonamide,
1-Acetyl-4-[(4-bromophenyl)sulfonyl]piperazine,
4-Bromo-N-methyl-N-(1-methylpiperidin-4-yl)benzenesulfonamide,
4-Bromo-N-[3-(dimethylamino)propyl]-N-methylbenzenesulfonamide,
4-Bromo-N-[2-(dimethylamino)ethyl]-N-ethylbenzenesulfonamide,
4-Bromo-N-[3-(4-methylpiperazin-1-yl)propyl]benzenesulfonamide,
1-[(4-Bromophenyl)sulfonyl]-4-ethylpiperazine,
4-Bromo-N-(2-pyrrolidin-1-ylethyl)benzenesulfonamide,
1-[(4-Bromophenyl)sulfonyl]-4-methyl-1,4-diazepane,
4-Bromo-N-[2-(-dimethylamino)propyl]benzenesulfonamide,
4-Bromo-N-[(1-ethylpyrrolidin-2-yl)methyl]benzenesulfonamide,
4-Bromo-N-[2-(diethylamino)ethyl]benzenesulfonamide,
4-Bromo-N-(2-pyridin-2-ylethyl)benzenesulfonamide,
4-Bromo-N-[3-(dimethylamino)propyl]benzenesulfonamide,
1-[(4-Bromophenyl)sulfonyl]-N,N-dimethylpyrrolidin-3-amine,
4-[(4-Bromophenyl)sulfonyl]morpholine,
4-Bromo-N-isopropyl-N-(2-methoxyethyl)benzenesulfonamide,
4-Bromo-N-(2-methoxy-1-methylethyl)benzenesulfonamide,
4-Bromo-N-[2-(dimethylamino)ethyl]benzamide,
4-Bromo-N-[2-(dimethylamino)ethyl]-N-methylbenzamide,
N-[2-Fluoro-4-[(4-methyl-1-piperazinyl)sulfonyl]phenyl}acetamide,
2-Methyl-4-[(4-methylpiperazin-1-yl)sulfonyl]aniline,
1-[(4-Bromo-3-methylphenyl)sulfonyl]-4-methylpiperazine,
2-Fluoro-4-[(4-methyl-1-piperazinyl)sulfonyl]benzenamine,
1-[(4-Bromo-3-fluorophenyl)sulfonyl]-4-methylpiperazine,
4-[(4-Methylpiperazin-1-yl)sulfonyl]-2-(trifluoromethyl)aniline,
1-{[4-Bromo-3-(trifluoromethyl)phenyl]sulfonyl}-4-methylpiperazine,
1-[(4-Bromo-2-fluoro-5-methylphenyl)sulfonyl]-4-methylpiperazine,
1-[(4-Bromo-2,5-dimethylphenyl)sulfonyl]-4-methylpiperazine,
1-[(4-Bromophenyl)sulfonyl]piperidine,
1-[(4-Bromophenyl)sulfonyl]pyrrolidine,
-1-[(4-Bromo-2,5-difluorophenyl)sulfonyl]piperidine,
1-[(4-Bromo-2,5-difluorophenyl)sulfonyl]pyrrolidine,
tert-Butyl 4-[(4-bromophenyl)sulfonyl]piperazine-1-carboxylate,
1-(4-Bromobenzoyl)-4-methylpiperazine,
3-(4-Bromophenoxy)-1-methylpyrrolidine,
tert-Butyl 4-[2-(4-bromophenoxy)ethyl]piperazine-1-carboxylate,
tert-Butyl 4-[2-(4-bromo-2,5-difluorophenoxy)ethyl]piperazine-1-carboxylate,
4-[2-(4-Bromo-2,5-difluorophenoxy)ethyl]morpholine,
1-[2-(4-Bromo-3,5-dimethylphenoxy)ethyl]-4-methylpiperazine,
1-[2-(4-Bromo-3-methylphenoxy)ethyl]-4-methylpiperazine,
1-[2-(4-Bromo-2,5-difluorophenoxy)ethyl]pyrrolidine,
5-Bromo-N,N-dimethylthiophene-2-sulfonamide,
tert-Butyl 4-(5-bromo-2-furoyl)piperazine-1-carboxylate,
3-Ethyl-4-[(4-methylpiperazin-1-yl)sulfonyl]phenylboronic acid,
4-[(4-Methylpiperazin-1-yl)sulfonyl]-3-(trifluoromethoxy)phenylboronic acid,
4-{[4-(tert-Butoxycarbonyl)piperazin-1-yl]sulfonyl}phenylboronic acid,
2,5-Difluoro-4-(piperidin-1-ylsulfonyl)phenylboronic acid,
2,5-Difluoro-4-(pyrrolidin-1-ylsulfonyl)phenylboronic acid,
4-(Pyrrolidin-1-ylsulfonyl)phenylboronic acid,
4-(Piperidin-1-ylsulfonyl)phenylboronic acid,
4-[(Dimethylamino)sulfonyl]phenylboronic acid,
4-((Methyl(-1-methylpyrrolidin-3-yl)amino)sulfonyl)phenylboronic acid,
4-((4-Acetylpiperazin-1-yl)sulfonyl)phenylboronic acid,
4-(((2-Dimethylamino)ethyl)(ethyl)amino)sulfonyl)phenylboronic acid,
4-((3-Dimethylamino)pyrrolidin-1-yl)sulfonyl)phenylboronic acid,
4-(((2-Dimethylamino)-1-methylethyl)amino)sulfonyl)phenylboronic acid,
-4-((3-Pyrrolidin-1-ylpropyl)amino)sulfonyl)phenylboronic acid,
4-((Methyl-(1-methylpiperidin-4-yl)amino)sulfonyl)phenylboronic acid,
4-(((Dimethylamino)propyl)(methyl)amino)sulfonyl)phenylboronic acid,
4-(Morpholin-4-ylsulfonyl)phenylboronic acid,
4-(((3-(4-Methylpiperazin-1-yl)propyl)amino)sulfonyl)phenylboronic acid,
4-((4-Ethylpiperazin-1-yl)sulfonyl)phenylboronic acid,
4-((2-Pyrrolidin-1-ylethyl)amino)sulfonyl)phenylboronic acid,
4-((4-Methyl-1,4-diazepan-1-yl)sulfonyl)phenylboronic acid,
4-(((2-Dimethylamino)propyl)amino)sulfonyl)phenylboronic acid,
4-((Isopropyl-(2-methoxyethyl)amino)sulfonyl)phenylboronic acid,
4-((((1-Ethylpyrrolidin-2-yl)amino)sulfonyl)phenylboronic acid,
4-(((2-Diethylamino)ethyl)amino)sulfonyl)phenylboronic acid,
4-(((2-Pyridin-2-ylethyl)amino)sulfonyl)phenylboronic acid,
4-(((2-Methoxy-1-methylethyl)amino)sulfonyl)phenylboronic acid,
4-(((3-Dimethylamino)propyl)amino)sulfonyl)phenylboronic acid,
tert-Butyl 4-[(dimethylamino)methyl]pyridin-3-ylcarbamate,
4-[(Dimethylamino)methyl]pyridin-3-amine,
4-(Pyrrolidin-1-ylmethyl)pyridin-3-amine,
4-(2-Pyrrolidin-1-ylethyl)pyridin-3-amine,
4-(3-Pyrrolidin-1-ylpropyl)pyridin-3-amine,
tert-Butyl 4-(pyrrolidin-1-ylmethyl)pyridin-3-ylcarbamate,
tert-Butyl 4-(2-pyrrolidin-1-ylethyl)pyridin-3-ylcarbamate,
tert-Butyl 4-(2-hydroxyethyl)pyridin-3-ylcarbamate,
tert-Butyl 4-(3-pyrrolidin-1-ylpropyl)pyridin-3-ylcarbamate,
tert-Butyl 4-(3-pyrrolidin-1-ylprop-1-ynyl)pyridin-3-ylcarbamate,
tert-Butyl 5-(3-pyrrolidin-1-ylprop-1-ynyl)pyridin-3-ylcarbamate,
-tert-butyl 4-[3-(dimethylamino)prop-1-ynyl]pyridin-3-ylcarbamate,
4-(3-Dimethylaminopropyl)pyridin-3-ylamine,
5-(3-Pyrrolidin-1-ylpropyl)pyridin-3-amine,
tert-Butyl 4-(3-hydroxyprop-1-ynyl)pyridin-3-ylcarbamate,
tert-Butyl 5-(3-hydroxyprop-1-ynyl)pyridin-3-ylcarbamate,
tert-Butyl 5-[3-(dimethylamino)prop-1-ynyl]pyridin-3-ylcarbamate,
tert-Butyl 5-bromopyridin-3-ylcarbamate,
tert-Butyl 5-[3-(dimethylamino)propyl]pyridin-3-ylcarbamate,
5-[3-(Dimethylamino)propyl]pyridin-3-amine or
Methyl 3-amino-6-{4-[(dimethylamino)sulfonyl]phenyl}pyrazine-2-carboxylate.

42. (canceled)

43. A compound which is 3-Amino-6-4{4-[(4-methylpiperazin-1-yl)sulfonyl]phenyl}-N-pyridin-3-ylpyrazine-2-carboxamide as a free base or a pharmaceutically acceptable salt thereof.

44. The compound according to claim 43 which is 3-Amino-6-4{4-[(4-methylpiperazin-1-yl)sulfonyl]phenyl}-N-pyridin-3-ylpyrazine-2-carboxamide hydrochloride.

45. A compound which is 3-Amino-6-(4{[2-methoxy-1-methylethyl)amino]sulfonyl}phenyl)-N-pyridin-3-ylpyrazine-2-carboxamide hydrochloride.

46. A compound which is 3-Amino-6-{2,5-difluoro-4-[(4-methylpiperazin-1-yl)sulfony]phenyl}-N-pyridin-3-ylpyrazine-2-carboxamide hydrochloride.

47. A compound which is 3-Amino-6-{3-fluoro-4-[(4-methylpiperazin-1-yl)sulfonyl]phenyl}-N-pyridin-3-ylpyrazine-2-carboxamide hydrochloride.

48. A compound which is 3-Amino-6-[4[4-methylpiperazin-1-yl)sulfonyl]-3-(trifluoromethoxy)phenyl]-N-pyridin-3-ylpyrazine-2-carboxamide as a free base or a pharmaceutically acceptable salt thereof.

49. A compound according to claim 36 wherein Z is N.

50. A compound which is
3-Amino-6-bromo-N-pyridin-3-ylpyrazine-2-carboxamide,
2-Amino-5-bromo-N-pyridin-3-ylnicotinamide,
2-Amino-5-bromo-N-[4-(pyrrolidin-1-ylmethyl)pyridin-3-yl]nicotinamide,
3-Amino-6-bromo-N-[4-(pyrrolidin-1-ylmethyl)pyridin-3-yl]pyrazine-2-carboxamide,
3-Amino-6-bromo-N-[4-(2-pyrrolidin-1-ylethyl)pyridin-3-yl]pyrazine-2-carboxamide,
3-Amino-6-bromo-N-{4-[(dimethylamino)methyl]pyridin-3-yl}pyrazine-2-carboxamide,
3-Amino-6-bromo-N-{5-[3-(dimethylamino)propyl]pyridin-3-yl}pyrazine-2-carboxamide,
3-Amino-6-bromo-N-[5-(3-pyrrolidin-1-ylpropyl)pyridin-3-yl]pyrazine-2-carboxamide, or
2-Amino-5-bromo-N-(3-pyridinyl)benzamide.

51. A compound which is
3-Amino-6-{4-[(dimethylamino)sulfonyl]phenyl}pyrazine-2-carboxylic acid,
tert-Butyl 4-formylpyridin-3-ylcarbamate,
3-Amino-6-[4-(pyrrolidin-1-ylsulfonyl)phenyl]pyrazine-2-carboxylic acid or
Methyl 3-amino-6-[4-(pyrrolidin-1-ylsulfonyl)phenyl]pyrazine-2-carboxylate.


SEQUENCE LISTING (1 sequence)

SEQ ID NO: 1 - protein, 14 aa, Unknown
  a biotinylated peptide substrate
AAEELDSRAGSPQL